(19) United States
(12) Patent Application Publication
(10) Pub. No.: US 2016/0311791 A1
(43) Pub. Date: Oct. 27, 2016

(54) NOVEL TRICYCLIC CALCIUM SENSING RECEPTOR ANTOGONISTS FOR THE TREATMENT OF OSTEOPOSOSIS

(71) Applicant: MERCK SHARP & DOHME CORP., Rahway, NJ (US)

(72) Inventors: Gui-Bai Liang, Scotch Plains, NJ (US); Changyou Shou, Princeton, NJ (US); Xianghong Huo, Changping District, Beijing (CN); Haisheng Wang, Changping, Beijing (CN)

(73) Assignee: Merck Sharp & Dohme Corp., Rahway, NJ (US)

(21) Appl. No.: 15/104,394

(22) PCT Filed: Dec. 17, 2014
(86) PCT No.: PCT/US14/70718

§ 371 Date: Jun. 14, 2016

(30) Foreign Application Priority Data
Dec. 20, 2013 (CN) PCT/CN2013/090147

(51) Int. Cl. C07D 307/93; C07D 409/12; C07C 323/32; C07C 217/28; C07D 307/79; C07D 333/54; C07D 407/12; C07D 405/12

(52) U.S. Cl. CPC C07D 307/93; C07D 407/12; C07D 409/12; C07D 405/12; C07C 217/28; C07D 307/79; C07D 333/54; C07C 323/32; C07C 2103/08; C07C 2102/08

(57) ABSTRACT

Novel tricyclic compounds of Formula (I) and pharmaceutically acceptable salts thereof are disclosed as useful for treating or preventing osteoporosis and similar conditions. The compounds are effective as calcium sensing receptor antagonists. Pharmaceutical compositions and methods of treatment are also included.

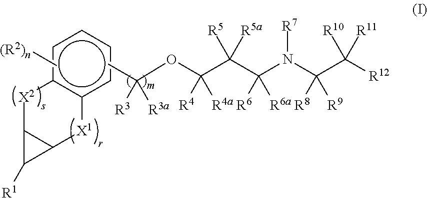

### FIELD OF THE INVENTION

The present invention relates to novel tricyclic compounds and salts thereof useful as calcium sensing receptor antagonists which contain a cyclopropyl carboxylic acid or carboxylic acid derivative (e.g., ester and amide) fused to a bicyclic ring. The present invention further relates to compositions containing such compounds, and methods of use thereof.

### BACKGROUND OF THE INVENTION

A variety of disorders in humans and other mammals involve or are associated with abnormal bone and mineral homeostasis. Such disorders include, but are not limited to, hypoparathyroidism, osteosarcoma, chondrosarcoma, periodontal disease, bone fracture healing, osteoarthritis, Paget's disease, osteopenia, glucocorticoid induced osteoporosis, osteomalacia, osteoporosis, metastatic bone disease, abnormally increased bone turnover and joint replacement. One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are major causes of morbidity and mortality in the elderly population. As many as 50% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment.

The calcium sensing receptor (CaSR) is a class C G-protein coupled receptor (GPCR), and is highly expressed on the surface of parathyroid cells; see, Brown et al., Bone 2009, 44, S201-S202. CaSR, in responses to rising concentration of circulating extracellular calcium ion, down regulates the production and secretion of parathyroid hormone (PTH); see, Tfelt-Hansen et al., Critical Reviews in Clinical Laboratory Sciences 2005, 42, 35. PTH is an 84-amino acid peptide, produced and secreted by the parathyroid glands, and PTH regulates calcium homeostasis through actions on kidney and bone; see, Marie, P. J., Bone 2010, 46, 571; Gowen et al., Journal of Clinical Investigation 2000, 105, 1595. Bone anabolic effects and increased bone strength following transient exposure to PTH have been well studied (see Rubin et al., Osteoporosis International 2002, 13, 267), and PTH 1-84 (Nycomed; see, Girotra et al., J. P. Reviews in Endocrine & Metabolic Disorders 2006, 7, 113) and the recombinant N-terminal PTH 1-34 amino acid fragment (Teriparatide; see Compston, J. Calcified Tissue International 2005, 77, 65) are FDA approved injectable agents for the treatment of osteoporosis. In search for orally efficacious bone anabolic agents, small molecule CaSR antagonists have been extensively studied in pharmaceutical industry in the past decade; see, Widler et al., Journal of Medicinal Chemistry 2010, 53, 2250; Marquis et al., Journal of Medicinal Chemistry 2009, 52, 6599; John et al., Bone 2011, 49, 233; Widler et al., Future Medicinal Chemistry 2011, 3, 535. It has been shown that CaSR antagonists can trigger the release of PTH and stimulate bone growth in animal models (see, Hoffman et al., Journal of Bone and Mineral Research 2008, 23, S336), and several clinical trials have been conducted, among them Ronacaleret (see, Fitzpatrick et al., Journal of Bone and Mineral Research 2008, 23, S50) from GSK, and ATF936 (see, John et al., supra) from Novartis. However, it has become clear that continuous infusion of PTH over an extended period of time only results in increased bone turnover and overall bone loss (see, Onyia et al., Journal of Cellular Biochemistry 2005, 95, 403). Thus, it would be useful to have additional novel CaSR antagonists. Particularly desirable would be to have select molecules with very short half lives to ensure the transient and rapid secretion of endogeneous PTH.

### SUMMARY OF THE INVENTION

The present invention addresses compounds represented by the formula:

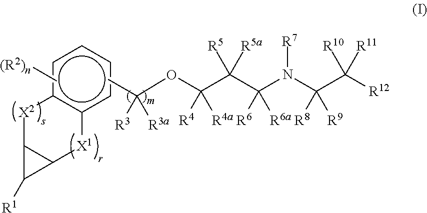

and pharmaceutically acceptable salts thereof. The present invention further relates to methods of treating osteoporosis and related diseases and conditions.

### DETAILED DESCRIPTION OF THE INVENTION

The present invention addresses the following compounds, compounds of (1)-(44):

(1) A compound of the formula (Ia):

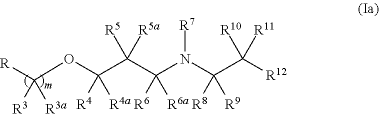

or a pharmaceutically acceptable salt thereof, wherein:

R is

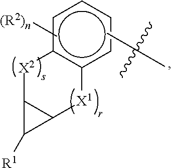

One of r and s is 0, and the other is 1,

X¹ when present, is selected from O, S, CR^{e}R^{f}, or OCR^{e}R^{f},

X² when present, is selected from O, S, CR^{e}R^{f}, or OCR^{e}R^{f},

R^{e} and R^{f} are each independently hydrogen or C₁₋₆ alkyl, wherein alkyl substituents are further optionally substituted with 1-4 halogen substituents,

R¹ is CO₂R^{a} or CONR^{b}R^{c},

R^{a} is hydrogen or C₁₋₆ alkyl,

R^{b} and R^{c} are each independently hydrogen, C₁₋₆ alkyl, or SO₂R^{d},

R^{d} is C₁₋₆ alkyl or a 3- to 6-membered cycloalkyl group optionally substituted with 1-4 substituents independently selected from: halogen or C₁₋₆alkyl, wherein alkyl substituents are further optionally substituted with 1-4 halogen substituents,

R² is halogen or C₁₋₆ alkyl, wherein alkyl substituents are further optionally substituted with 1-4 halogen substituents,

n is 0, 1, 2, or 3,

R³ and R^{3a} are each independently hydrogen or C₁₋₆ alkyl, wherein alkyl substituents are further optionally substituted with 1-4 halogen substituents,

m is 0, 1, 2, or 3,

R⁴ and R^{4a} are each independently hydrogen or C₁₋₆ alkyl, wherein alkyl substituents are further optionally substituted with 1-4 halogen substituents,

R⁵ and R^{5a} are each independently hydrogen, hydroxyl, or C₁₋₆ alkyl, wherein alkyl substituents are further optionally substituted with 1-4 halogen substituents,

R⁶ and R^{6a} are each independently hydrogen or C₁₋₆ alkyl, wherein alkyl substituents are further optionally substituted with 1-4 halogen substituents,

R⁷, R⁸ and R⁹ are each independently hydrogen or C₁₋₆ alkyl, or R⁷ is hydrogen or C₁₋₆ alkyl and R⁸ and R⁹ together form a 3- to 6-membered cycloalkyl group optionally substituted with 1-4 substituents independently selected from: halogen or C₁₋₆alkyl, or R⁷ and R⁸ together form a 5- to 6-membered heterocyclic group optionally substituted with 1-4 substituents independently selected from: halogen or C₁₋₆alkyl and R⁹ is hydrogen or C₁₋₆ alkyl,

R¹⁰ and R¹¹ are each independently hydrogen or C₁₋₆ alkyl, wherein alkyl substituents are further optionally substituted with 1-4 halogen substituents, or R¹⁰ and R¹¹ together form an oxo group, and

R¹² is a 6- to 10-membered aryl group, a 5- to 10-membered heteroaryl group, a 5- to 7-membered cycloalkyl group, a 5- to 7-membered heterocyclic group, —(CH₂)₀₋₃—O—(CH₂)₀₋₁-6- to 10-membered aryl, —(CH₂)₁₋₃-6- to 10-membered aryl, —(CH₂)₀₋₃—S—(CH₂)₀₋₁-6- to 10-membered aryl, wherein aryl, heteroaryl, cycloalkyl, heterocyclic group optionally substituted with 1-4 substituents independently selected from: halogen, hydroxyl, oxo, C₁₋₆alkyl, C₁₋₆alkylOC₁₋₆alkyl, C₁₋₆alkoxy, CN, C(O)₁₋₂C₁₋₆alkyl, C₁₋₆alkylC(O)₁₋₂C₁₋₆alkyl, or S(O)₀₋₂C₁₋₆alkyl, wherein alkyl substituents are further optionally substituted with 1-4 halogen substituents.

(2) A compound of (1), wherein:

R is

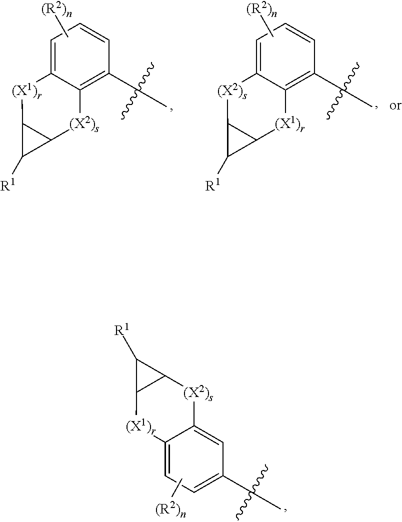

X¹ is O, S, CR^{e}R^{f}, or OCR^{e}R^{f} where the O atom in OCR^{e}R^{f} is directly attached to the phenyl ring of the R group, and R^{e} and R^{f} as defined above,
r is 1, and

s is 0, or a pharmaceutically acceptable salt thereof.

(3) A compound of (1) or (2), wherein:

R is

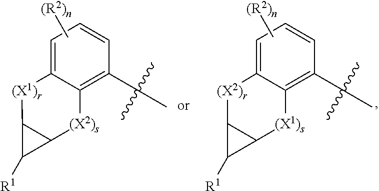

X¹ is O, S, CR^{e}R^{f}, or OCR^{e}R^{f} where the O atom in OCR^{e}R^{f} is directly attached to the phenyl ring of the R group, and Re and R^{f} as defined above,

r is 1, and

s is 0, or a pharmaceutically acceptable salt thereof.

(4) A compound of any of (1)-(3), wherein:

R is

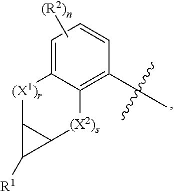

X¹ is O, S, CR^{e}R^{f}, or OCR^{e}R^{f} where the O atom in OCR^{e}R^{f} is directly attached to the phenyl ring of the R group, and R^{e} and R^{f} as defined above,

r is 1, and

s is 0, or a pharmaceutically acceptable salt thereof.

(5) A compound of (1), wherein r is 1 and s is 0, or a pharmaceutically acceptable salt thereof.

(6) A compound of (1), wherein r is 0 and s is 1, or a pharmaceutically acceptable salt thereof.

(7) A compound of any of (1)-(6), wherein:

X¹ when present, is selected from O, S, CH₂, or OC(CH₃)₂, and

X² when present, is selected from O, S, CH₂, or OC(CH₃)₂, or a pharmaceutically acceptable salt thereof.

(8) A compound of any of (1)-(7), wherein:

X¹ when present, is selected from O or CH₂, and

X² when present, is selected from O or CH₂, or a pharmaceutically acceptable salt thereof.

(9) A compound of any of (1)-(8), wherein:

X¹ when present, is oxygen, and

X² when present, is oxygen, or a pharmaceutically acceptable salt thereof.

(10) A compound of any of (1)-(9), wherein:

R¹ is CO₂H, CO₂C₁₋₆alkyl, or CONHS(O)₂R^{d}, and

R^{d} is a 3- to 6-membered cycloalkyl group optionally substituted with 1-4 substituents independently selected from: halogen or C₁₋₆alkyl, or a pharmaceutically acceptable salt thereof.

(11) A compound of any of (1)-(10), wherein R¹ is CO₂H, CO₂CH₂CH₃, or CONHS(O)₂cyclopropylCH₃, or a pharmaceutically acceptable salt thereof.

(12) A compound of any of (1)-(11), wherein R¹ is CO₂H, or a pharmaceutically acceptable salt thereof.

(13) A compound of any of (1)-(12), wherein R² is halogen or CH₃, or a pharmaceutically acceptable salt thereof.

(14) A compound of any of (1)-(13), wherein R² is halogen, or a pharmaceutically acceptable salt thereof.

(15) A compound of any of (1)-(14), wherein R² is fluoro or chloro, or a pharmaceutically acceptable salt thereof.

(16) A compound of any of (1)-(13), wherein R² is CH₃, or a pharmaceutically acceptable salt thereof.

(17) A compound of any of (1)-(16), wherein n is 0 or 1, or a pharmaceutically acceptable salt thereof.

(18) A compound of any of (1)-(17), wherein n is 0, or a pharmaceutically acceptable salt thereof.

(19) A compound of any of (1)-(18), wherein R³ and R^{3a} are each independently hydrogen or CH₃, or a pharmaceutically acceptable salt thereof.

(20) A compound of any of (1)-(19), wherein one of R³ and R^{3a} is hydrogen, and the other is CH₃, or a pharmaceutically acceptable salt thereof.

(21) A compound of any of (1)-(20), wherein m is 0 or 1, or a pharmaceutically acceptable salt thereof.

(22) A compound of any of (1)-(21), wherein m is 1, or a pharmaceutically acceptable salt thereof.

(23) A compound of any of (1)-(22), wherein R⁴ and R^{4a} are each independently hydrogen or CH₃, or a pharmaceutically acceptable salt thereof.

(24) A Compound of any of (1)-(23), wherein R⁴ and R^{4a} are both hydrogen, or a pharmaceutically acceptable salt thereof.

(25) A compound of any of (1)-(24), wherein R⁵ and R^{5a} are each independently hydrogen, hydroxyl, or CH₃, or a pharmaceutically acceptable salt thereof.

(26) A compound of any of (1)-(25), wherein one of R⁵ and R^{5a} is hydroxyl, and the other is hydrogen, or a pharmaceutically acceptable salt thereof.

(27) A compound of any of (1)-(26), wherein R⁶ and R^{6a} are each independently hydrogen or CH₃, or a pharmaceutically acceptable salt thereof.

(28) A compound of any of (1)-(27), wherein R⁶ and R^{6a} are both hydrogen, or a pharmaceutically acceptable salt thereof.

(29) A compound of any of (1)-(28), wherein R⁷, R⁸ and R⁹ are each independently hydrogen or CH₃, or R⁷ is hydrogen or CH₃ and R⁸ and R⁹ together form a 3- to 5-membered cycloalkyl group optionally mono- or di-substituted with CH₃, or R⁷ and R⁸ together form a pyrrolidine or piperidine group optionally mono- or di-substituted with CH₃ and R⁹ is hydrogen or CH₃, or a pharmaceutically acceptable salt thereof.

(30) A compound of any of (1)-(29), wherein R⁷ is hydrogen and R⁸ and R⁹ are both CH₃, or R⁷ is hydrogen and R⁸ and R⁹ together form a 3- to 4-membered cycloalkyl group, or R⁷ and R⁸ together form a 5-membered cycloalkyl group and R⁹ is hydrogen, or a pharmaceutically acceptable salt thereof.

(31) A compound of any of (1)-(30), wherein R⁷ is hydrogen, while R⁸ and R⁹ are both CH₃, or a pharmaceutically acceptable salt thereof.

(32) A compound of any of (1)-(31), wherein R¹⁰ and R¹¹ are each independently hydrogen or CH₃, or a pharmaceutically acceptable salt thereof.

(33) A compound of any of (1)-(32), wherein R¹⁰ and R¹¹ are both hydrogen, or a pharmaceutically acceptable salt thereof.

(34) A compound of any of (1)-(33), wherein R¹² is phenyl group, naphthyl group, indanyl group, quinolyl group, benzothienyl group, dihydrobenzothienyl group, benzofuranyl group, dihydrobenzofuranyl group, benzodioxolanyl group, benzodioxanyl group, tetrahydroisoquinolyl group, —(CH₂)₀₋₁—S—(CH₂)₀₋₁-phenyl, —(CH₂)₀₋₁—O—(CH₂)₀₋₁-phenyl, or —(CH₂)₂-phenyl, optionally substituted with 1-4 substituents independently selected from: halogen, C₁₋₆alkyl, C₁₋₆alkoxy, or S(O)₀₋₂C₁₋₆alkyl, where said C₁₋₆alkyl is optionally substituted by 1-6 halogens, or a pharmaceutically acceptable salt thereof.

(35) A compound of any of (1)-(34), wherein R¹² is phenyl group, naphthyl group, indanyl group, quinolyl group, benzothienyl group, dihydrobenzothienyl group, benzofuranyl group, dihydrobenzofuranyl group, benzodioxolanyl group, benzodioxanyl group, tetrahydroisoquinolyl group, —(CH₂)₀₋₁—S—(CH₂)₀₋₁-phenyl, —(CH₂)₀₋₁—O—(CH₂)₀₋₁-phenyl, or —(CH₂)₂-phenyl, optionally substituted with 1-4 substituents independently selected from: halogen, CH₃, CF₃, OCH₃, or S(O)₀₋₁CH₃, or a pharmaceutically acceptable salt thereof.

(36) A compound of any of (1)-(35), wherein R⁴, R^{4a}, R⁵, R⁶, and R^{ha} are all hydrogen, or a pharmaceutically acceptable salt thereof.

(37) A compound of any of (36), wherein R^{5a} is hydroxyl, or a pharmaceutically acceptable salt thereof.

(38) A compound of any of (36) or (37), wherein R³ is CH₃, or a pharmaceutically acceptable salt thereof.

(39) A compound of any of (36)-(38), wherein R^{3a} is hydrogen, or a pharmaceutically acceptable salt thereof.

(40) A compound of any of (36)-(39), wherein R⁷ is hydrogen, or a pharmaceutically acceptable salt thereof.

(41) A compound of any of (36)-(40), wherein R⁸ and R⁹ are both CH₃, or a pharmaceutically acceptable salt thereof.

(42) A compound of any of (36)-(41), wherein R¹⁰ and R¹¹ are both hydrogen, or a pharmaceutically acceptable salt thereof.

(43) A compound of (I) which is disclosed in Examples 1-24 or is:
5-(1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
5-(1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
5-(1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
3-(1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
3-(1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
6-(1-((R)-3-((1-(4-chloro-3-fluorophenyl)-2-methylpropan-2-yl)amino)-2-hydroxypropoxy)ethyl)-3-methyl-1a,6b-dihydro-1H-cyclopropa[b]benzofuran-1-carboxylic acid;
6-(1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
6-(1-((R)-3-((1-(2,3-dihydro-1H-inden-2-yl)-2-methylpropan-2-yl)amino)-2-hydroxypropoxy)ethyl)-3-methyl-1a,6b-dihydro-1H-cyclopropa[b]benzofuran-1-carboxylic acid;
6-((R)-1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
3-(1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
5-((R)-3-((1-(4-chloro-3-fluorophenyl)-2-methylpropan-2-yl)amino)-2-hydroxypropoxy)-1a,6b-dihydro-1H-cyclopropa[b]benzofuran-1-carboxylic acid;
5-((R)-2-hydroxy-3-((2-methyl-1-(naphthalen-2-yl)propan-2-yl)amino)propoxy)-1a,6b-dihydro-1H-cyclopropa[b]benzofuran-1-carboxylic acid;
5-((R)-3-((1-(2,3-dihydro-1H-inden-2-yl)-2-methylpropan-2-yl)amino)-2-hydroxypropoxy)-1a,6b-dihydro-1H-cyclopropa[b]benzofuran-1-carboxylic acid;
6-(1-((R)-3-((1-(4-chloro-3-fluorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
6-(1-((R)-3-((1-(2,3-dihydro-1H-inden-2-yl)-2-methylpropan-2-yl)amino)-2-hydroxypropoxy)ethyl)-3-methyl-1a,6b-dihydro-1H-cyclopropa[b]benzofuran-1-carboxylic acid;
6-(1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-3-((1-(4-chloro-3-fluorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(2,3-dihydro-1H-inden-2-yl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-3-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1R,1aR,6bS)-3-((R)-1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1R,1aR,6bS)-3-((R)-1-((R)-3-((1-(3,4-dimethylphenyl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(4-chloro-2-fluorophenyl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-1-(naphthalen-1-yl)propan-2-yl)amino)propoxy)ethyl)-3-methyl-1a,6b-dihydro-1H-cyclopropa[b]benzofuran-1-carboxylic acid;
(1R,1aR,6bS)-6-((R)-1-((R)-3-((1-(2,3-dihydro-1H-inden-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-3-((1-(4-chloro-3-fluorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
(1R,1aR,6bS)-6-((R)-1-((R)-3-((1-(2,3-dihydro-1H-inden-2-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-6-((R)-1-((R)-3-((1-(2-chloro-4-methoxyphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-3-((R)-1-((R)-3-((1-(2,3-dihydro-1H-inden-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(3-fluoro-4-(methylthio)phenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(2,3-dihydro-1H-inden-5-yl)-2-methylpropan-2-yl)amino)-2-hydroxypropoxy)ethyl)-1a,6b-dihydro-1H-cyclopropa[b]benzofuran-1-carboxylic acid;
6-(1-((R)-2-hydroxy-3-((2-methyl-1-(naphthalen-2-yl)propan-2-yl)amino)propoxy)ethyl)-1a,6b-dihydro-1H-benzo[b]cyclopropa[d]thiophene-1-carboxylic acid;
(1S,1aS,6bR)-3-((R)-1-((R)-3-((1-(benzo[b]thiophen-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydrobenzo[b][1,4]dioxin-6-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3-fluoro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((1R)-1-((2R)-3-((1-(3-fluoro-4-(methylsulfinyl)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydrobenzo[b]thiophen-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzo[b]thiophen-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-4-(phenylthio)butan-2-yl)amino)propoxy)ethyl)-3-methyl-1a,6b-dihydro-1H-cyclopropa[b]benzofuran-1-carboxylic acid;
(1R,1aR,6aS)-2-((R)-1-((R)-2-hydroxy-3-((2-methyl-1-(naphthalen-2-yl)propan-2-yl)amino)propoxy)ethyl)-1,1a,6,6a-tetrahydrocyclopropa[a]indene-1-carboxylic acid;
(1S,1aS,6bR)-6-((R)-1-((R)-3-((1-(3-chloro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-5-chloro-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-5-chloro-6-((R)-1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
3-fluoro-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
3-chloro-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-5-fluoro-3-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-5-chloro-3-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6aR)-2-((R)-1-((R)-2-hydroxy-3-((2-methyl-1-(naphthalen-2-yl)propan-2-yl)amino)propoxy)ethyl)-1,1a,6,6a-tetrahydrocyclopropa[a]indene-1-carboxylic acid;
(1R,1aR,6aS)-5-((R)-1-((R)-2-hydroxy-3-((2-methyl-1-(naphthalen-2-yl)propan-2-yl)amino)propoxy)ethyl)-1,1a,6,6a-tetrahydrocyclopropa[a]indene-1-carboxylic acid;
(1S,1aS,6aR)-5-((R)-1-((R)-2-hydroxy-3-((2-methyl-1-(naphthalen-2-yl)propan-2-yl)amino)propoxy)ethyl)-1,1a,6,6a-tetrahydrocyclopropa[a]indene-1-carboxylic acid;
7-(1-((R)-2-hydroxy-3-((2-methyl-1-(naphthalen-2-yl)propan-2-yl)amino)propoxy)ethyl)-2,2,4-trimethyl-1,1a,2,7b-tetrahydrocyclopropa[c]chromene-1-carboxylic acid;
(1S,1aS,6bR)-ethyl 6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylate;
(1S,1aS,6bR)-ethyl 6-((R)-1-((R)-3-((1-(3-fluoro-4-(methylthio)phenyl)-2-methylpropan-2-yl)amino)-2-hydroxypropoxy)ethyl)-3-methyl-1a,6b-dihydro-1H-cyclopropa[b]benzofuran-1-carboxylate;
(1S,1aS,6bR)-6-((R)-1-((R)-2-hydroxy-3-((2-methyl-1-(naphthalen-2-yl)propan-2-yl)amino)propoxy)ethyl)-N-((1-methylcyclopropyl)sulfonyl)-1a,6b-dihydro-1H-cyclopropa[b]benzofuran-1-carboxamide;
(1S,1aS,6bR)-3-((R)-1-((R)-2-hydroxy-3-((2-methyl-1-(naphthalen-2-yl)propan-2-yl)amino)propoxy)ethyl)-N-((1-methylcyclopropyl)sulfonyl)-1a,6b-dihydro-1H-cyclopropa[b]benzofuran-1-carboxamide;
(1S,1aS,6bR)-6-((R)-1-((R)-3-((1-(3-fluoro-4-methylphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3,4-dimethylphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzo[b]thiophen-2-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzofuran-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((1R)-1-((2R)-3-(2-benzylpyrrolidin-1-yl)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(2,3-dihydro-1H-inden-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzo[d][1,3]dioxol-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzo[b]thiophen-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(2-fluoro-4-methoxyphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3,4-dichlorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-5-phenylpentan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-4-phenoxybutan-2-yl)amino)propoxy)ethyl)-3-methyl-1a,6b-dihydro-1H-cyclopropa[b]benzofuran-1-carboxylic acid;
(1S,1aS,6bR)-6-((1R)-((2R)-3-(2-benzyl-2-methylpyrrolidin-1-yl)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3,4-difluorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(4-fluoro-3-(trifluoromethyl)phenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(2,4,5-trifluorophenyl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(2,4,6-trifluorophenyl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(4-(methylthio)phenyl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(quinolin-6-yl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-3-((1-(1,1-dioxido-2,3-dihydrobenzo[b]thiophen-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzylthio)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((4-((4-fluorophenyl)thio)-2-methylbutan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-((4-fluorophenyl)thio)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3-chloro-4-(methylthio)phenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(3-methyl-4-(methylthio)phenyl)propan-2-yl)amino)propoxy)ethyl)-3-methyl-1a,6b-dihydro-1H-cyclopropa[b]benzofuran-1-carboxylic acid;
(1R,1aR,6bS)-6-((R)-1-((R)-3-((1-(3-fluoro-4-methylphenyl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-3-((1-(3-fluoro-4-methoxyphenyl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-2-hydroxy-3-((1-(naphthalen-2-ylmethyl)cyclopropyl)amino)propoxy)ethyl)-3-methyl-1a,6b-dihydro-1H-cyclopropa[b]benzofuran-1-carboxylic acid;
(1R,1aR,6bS)-6-((R)-1-((R)-2-hydroxy-3-((1-(naphthalen-2-ylmethyl)cyclobutyl)amino)propoxy)ethyl)-3-methyl-1a,6b-dihydro-1H-cyclopropa[b]benzofuran-1-carboxylic acid;
(1R,1aR,6bS)-6-((R)-1-((R)-3-((1-(2,4-dichlorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-3-((1-(3,4-dichlorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(3,4-dimethylphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(benzofuran-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(benzo[d][1,3]dioxol-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydrobenzo[b][1,4]dioxin-6-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2-fluoro-4-methoxyphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(3,4-dichlorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-1-(4-(methylthio)phenyl)propan-2-yl)amino)propoxy)ethyl)-1a,6b-dihydro-1H-cyclopropa[b]benzofuran-1-carboxylic acid;
(1S,1aS,6bR)-6-((R)-1-((R)-3-((1-(2-chloro-4-methoxyphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-4-(phenylthio)butan-2-yl)amino)propoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-1-(3-methyl-4-(methylthio)phenyl)propan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(3-fluoro-4-methylphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(3,4-dimethylphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(benzofuran-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(benzo[d][1,3]dioxol-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(2,3-dihydrobenzo[b][1,4]dioxin-6-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(2-fluoro-4-methylphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(2-fluoro-4-methoxyphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(3,4-dichlorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-2-hydroxy-3-((2-methyl-5-phenylpentan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(3,4-difluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((1R)-1-((2R)-2-hydroxy-3-((2-methyl-1-(4-(methylsulfinyl)phenyl)propan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(3-fluoro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((1R)-1-((2R)-3-((1-(3-fluoro-4-(methylsulfinyl)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(2-chloro-4-methoxyphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-2-hydroxy-3-((2-methyl-4-(phenylthio)butan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(3-chloro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-2-hydroxy-3-((2-methyl-1-(3-methyl-4-(methylthio)phenyl)propan-2-yl)amino)propoxy)ethyl)-1a,6b-dihydro-1H-cyclopropa[b]benzofuran-1-carboxylic acid;
(1R,1aR,6bS)-3-((R)-1-((R)-3-((1-(benzyloxy)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(3,4-dihydroisoquinolin-2(1H)-yl)-2-methyl-1-oxopropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(3,4-dihydroisoquinolin-2(1H)-yl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(3-fluoro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1R,1aR,6aS)-2-((R)-1-((R)-3-((1-(benzofuran-5-yl)-2-methylpropan-2-yl)amino)-2-hydroxypropoxy)ethyl)-1,1a,6,6a-tetrahydrocyclopropa[a]indene-1-carboxylic acid;
(1R,1aR,6aS)-2-((R)-1-((R)-3-((1-(2,3-dihydro-1H-inden-5-yl)-2-methylpropan-2-yl)amino)-2-hydroxypropoxy)ethyl)-1,1a,6,6a-tetrahydrocyclopropa[a]indene-1-carboxylic acid;
(1R,1aR,6aS)-2-((R)-1-((R)-3-((1-(benzo[b]thiophen-5-yl)-2-methylpropan-2-yl)amino)-2-hydroxypropoxy)ethyl)-1,1a,6,6a-tetrahydrocyclopropa[a]indene-1-carboxylic acid;
(1R,1aR,6aS)-2-((R)-1-((R)-3-((1-(3-fluoro-4-(methylthio)phenyl)-2-methylpropan-2-yl)amino)-2-hydroxypropoxy)ethyl)-1,1a,6,6a-tetrahydrocyclopropa[a]indene-1-carboxylic acid;
(1S,1aS,6bR)-6-((R)-1-((R)-3-((1-(benzofuran-5-yl)-2-methylpropan-2-yl)amino)-2-hydroxypropoxy)ethyl)-5-chloro-3-methyl-1a,6b-dihydro-1H-cyclopropa[b]benzofuran-1-carboxylic acid;
(1S,1aS,6bR)-6-((R)-1-((R)-3-((1-(benzo[b]thiophen-5-yl)-2-methylpropan-2-yl)amino)-2-hydroxypropoxy)ethyl)-5-chloro-3-methyl-1a,6b-dihydro-1H-cyclopropa[b]benzofuran-1-carboxylic acid;
(1S,1aS,6bR)-5-chloro-6-((R)-1-((R)-3-((1-(2,3-dihydrobenzo[b][1,4]dioxin-6-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-5-chloro-6-((R)-1-((R)-3-((1-(2-fluoro-4-methoxyphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-5-chloro-6-((R)-1-((R)-3-((1-(3-fluoro-4-(methylthio)phenyl)-2-methylpropan-2-yl)amino)-2-hydroxypropoxy)ethyl)-3-methyl-1a,6b-dihydro-1H-cyclopropa[b]benzofuran-1-carboxylic acid; or
a pharmaceutically acceptable salt thereof.

(44) A compound which is (R)-3′-(3-((1-(2,3-dihydro-1H-inden-2-yl)-2-methylpropan-2-yl)amino)-2-hydroxypropoxy)-4′-fluoro-[1,1′-biphenyl]-4-carboxylic acid or a pharmaceutically acceptable salt thereof.

The compounds of the present invention are further described herein using the terms defined below unless otherwise specified.

“Alkyl”, as well as other groups having the prefix “alk”, such as alkoxy, and the like, means carbon chains which may be linear or branched, or combinations thereof, containing the indicated number of carbon atoms. In specific embodiments, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like.

“Alkoxy” refers to an alkyl group linked to oxygen.

“Aryl”, alone or in combination, relates to a phenyl, naphthyl, indanyl group, or a phenyl ring fused to a cycloalkyl or a heterocycle group in which the point of attachment of the aryl group is on the aromatic portion.

“Cycloalkyl” means a saturated cyclic hydrocarbon radical having the number of carbon atoms designated. In specific embodiments, 3-7 carbon atoms are intended. “Cycloalkyl” also includes a monocyclic ring fused to an aryl group in which the point of attachment of the cycloalkyl group is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Heteroaryl” means an aromatic or partially aromatic cyclic ring structure in which one or more atoms in the ring, the heteroatom(s), is an element other than carbon. “Partially aromatic” refers to multi-cyclic fused ring groups where at least one but not all rings are aromatic, such as a benzodioxole group. Heteroatoms are typcially oxygen (“O”), sulfur (“S”) or nitrogen (“N”) atoms. Examples of heteroaryl groups include: pyrrolyl or pyrrole, isoxazolyl or isoxazole, isothiazolyl or isothiazole, pyrazolyl or pyrazole, pyridyl, oxazolyl or oxazole, oxadiazolyl or oxadiazole, thiadiazolyl or thiadiazole, thiazolyl or thiazole, imidazolyl or imidazole, triazolyl or triazole, tetrazolyl or tetrazole, furyl, triazinyl, thienyl, pyrimidyl, benzisoxazolyl or benzisoxazole, benzoxazolyl or benzoazole, benzothiazolyl or benzothiazole, benzothiadiazolyl or benzothiadiazole, dihydrobenzofuranyl or dihydrobenzofurane, indolinyl or indoline, pyridazinyl or pyridazine, indazolyl or indazole, isoindolyl or isoindole, dihydrobenzothienyl, indolizinyl or indolizine, cinnolinyl or cinnoline, phthalazinyl or phthalazine, quinazolinyl or quinazoline, naphthyridinyl or naphthyridine, carbazolyl or carbazole, benzodioxolyl or benzodioxole, quinoxalinyl or quinoxaline, purinyl or purine, furazanyl or furazane, isobenzylfuranyl or isobenzylfurane, benzimidazolyl or benzimidazole, benzofuranyl or benzofurane, benzothienyl or benzothiene, quinolyl or quinoline, oxo-dihydroqunoline, indolyl or indole, oxindole, isoquinolyl or isoquinoline, dibenzofuranyl or dibenzofurane, and the like.

“Heterocycle” or “heterocyclic” refers to a saturated cyclic ring structure in which one or more atoms in the ring, the heteroatom(s), is an element other than carbon. Heteroatoms are typcially oxygen (“O”), sulfur (“S”) or nitrogen (“N”) atoms. “Heterocycle” or “heterocyclic” also includes a monoheterocyclic ring fused to an aryl group in which the point of attachment is on the non-aromatic portion.

“Halogen” includes fluorine, chlorine, bromine and iodine.

Unless expressly depicted or described otherwise, variables depicted in a structural formula with a “floating” bond, such as substituent —(R²)ₙ, are permitted on any available carbon atom in the ring to which each is attached.

Substitution, where applicable, may be on any available carbon atom that results in a stable structure. Furthermore, where language indicates that certain groups or substituents (e.g., alkyl groups or substituents) are further optionally substituted, that language includes all groups or substituents having that particular group or substituent as a component thereof. For example, use of the language “wherein alkyl substituents are further optionally substituted” indicates that any substituents possessing an alkyl component(s) (e.g., (CH₂)₀₋₃—O—(CH₂)₀₋₁-6-10-membered aryl, C₁₋₆alkoxy and C₁₋₆alkylC₁₋₆alkyl) can be substituted in the alkyl group(s) thereof.

Also, number ranges where provided (e.g., 1-6, 0-3, etc.) expressly include each and every integer/whole number in that range as a discrete embodiment.

Atoms of the compounds described herein may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of any of (1)-(44). For example, different isotopic forms of hydrogen (H) include protium (¹H) and deuterium (²H). Protium is the predominant hydrogen isotope found in nature. Enriching for deuterium may yield certain therapeutic advantages, such as increasing in vivo half-life or reducing dosage requirements, or may provide a compound useful as a standard for characterization of biological samples. Isotopically-enriched compounds of any of (1)-(44) described herein can be prepared without undue experimentation by conventional techniques well known to those skilled in the art or by processes analogous to those described in the Examples herein using appropriate isotopically-enriched reagents and/or intermediates.

Individual tautomers of the compounds of any of (1)-(44), as well as mixtures thereof, are encompassed herein. Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers.

Compounds described herein may contain an asymmetric center and may thus exist as enantiomers. Where the compounds according to the invention possess two or more asymmetric centers, they may additionally exist as diastereomers. When bonds to the chiral carbon are depicted as straight lines in the formulas of the invention, it is understood that both the (R) and (S) configurations of the chiral carbon, and hence both enantiomers and mixtures thereof, are embraced. The present invention includes all such possible stereoisomers as substantially pure resolved enantiomers, racemic mixtures thereof, as well as mixtures of diastereomers. Except where otherwise specified, the formulae encompassing compounds of the present invention are shown without a definitive stereochemistry at certain positions. The present invention therefore may be understood to include all stereoisomers of compounds of any of (1)-(44) and pharmaceutically acceptable salts thereof.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of any of (1)-(44) may be obtained by stereospecific synthesis using optically pure starting materials or reagents of known configuration.

Furthermore, some of the crystalline forms for compounds of the present invention may exist as polymorphs and as such are intended to be included in the present invention. In addition, some of the compounds of the instant invention may form solvates with water or common organic solvents. Solvates, and in particular, the hydrates of the compounds of any of (1)-(44) are also included in the present invention.

The term “pharmaceutically acceptable salt” refers to a salt prepared from pharmaceutically acceptable non-toxic bases or acids including inorganic bases or acids and organic bases or acids.

Salts of basic compounds encompassed within the term “pharmaceutically acceptable salt” refer to non-toxic salts of the compounds described herein which are generally prepared by reacting the free base with a suitable organic or inorganic acid. Representative salts of basic compounds described herein include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, formate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, palmitate, pamoate (embonate), pantothenate, phosphate/diphosphate, polygalacturonate, salicylate, stearate, sulfate, subacetate, succinate, tannate, tartrate, teoclate, tosylate, triethiodide and valerate. Furthermore, where the compounds described herein carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. In particular embodiments, the salt is selected from ammonium, calcium, magnesium, potassium, or sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

The compounds of the present invention have bone anabolic activity, and therefore they are useful as therapeutic agents and/or preventive agents for diseases or disorders associated with abnormal bone and mineral homeostasis including but not limited to osteoporosis as well as hypoparathyroidism, osteosarcoma, chondrosarcoma, periodontal disease, bone fracture healing, osteoarthritis, Paget's disease, osteopenia, glucocorticoid-induced osteoporosis, osteomalacia, metastatic bone disease, abnormally increased bone turnover or joint replacement.

The calcium sensing receptor antagonist activity and bone anabolic properties of the compounds of Formula I and Ia may be determined according to methods available in the art for determining the functional response of cells regulated by the calcium sensing receptor, including but not limited to the Fluorescent Imaging Plate Reader (FLIPR®, Molecular Devices Corporation, Sunnyvale, Calif.) assay, parathyroid hormone (“PTH”) secretion by parathyroid cells, calcitonin secretion by C-cells, bone reabsorption by osteoclasts and Ca²⁺ secretion by kidney cells.

The present invention also relates to pharmaceutical compositions comprising compounds of any of (1)-(44) and a pharmaceutically acceptable carrier. The pharmaceutical compositions of the present invention comprise a compound of any of (1)-(44) as an active ingredient, as well as a pharmaceutically acceptable carrier and optionally other therapeutic ingredients.

A pharmaceutical composition may also comprise a prodrug, or a pharmaceutically acceptable salt thereof, if a prodrug is administered.

The compositions are typically suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the condition being treated and on the particular active ingredient selected. They may be conveniently presented in unit dosage form and prepared by any of the methods well-known in the art.

In practical use, compounds of any of (1)-(44) can be combined as the active ingredient in intimate admixture with the pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage form. Solid pharmaceutical carriers are therefore typically employed. If desired, tablets may be coated by standard aqueous or nonaqueous techniques. Such compositions and preparations typically comprise at least about 0.1 percent of active compound, the remainder of the composition being the carrier. The percentage of active compound in these compositions may, of course, be varied and is conveniently between about 2 percent to about 60 percent of the weight of the dosage form. The amount of active compound in such therapeutically useful compositions is such that an effective dosage will be delivered.

Alternatively, the active compound can be administered intranasally as, for example, in the form of liquid drops or a spray.

The tablets, capsules and the like also typically contain a binder. Examples of suitable binders include gum tragacanth, acacia, gelatin and a synthetic or semisynthetic starch derivative, such as hydroxypropylmethylcellulose (HPMC); excipients such as dicalcium phosphate; a disintegrating agent such as corn starch, potato starch, alginic acid; a lubricant such as magnesium stearate; and in some instances, a sweetening agent such as sucrose, lactose or saccharin. When the dosage form employed is a capsule, it may contain, in addition to the components described above, a liquid carrier such as fatty oil.

Various other materials may be present as coatings or to modify the physical form of the dosage unit. For instance, tablets may be coated with shellac, sugar or both. Syrups and elixirs typically contain, in addition to the active ingredient, sucrose as a sweetening agent, methyl or propylparabens as a preservative, a dye and a flavoring such as cherry or orange flavor.

The compounds of any of (1)-(44) may also be administered parenterally. Solutions or suspensions of these active compounds can be prepared in water, saline or another biocompatible vehicle, suitably mixed with a surfactant, buffer, and the like. Dispersions can also be prepared in glycerol, liquid polyethylene glycols and mixtures thereof in an oil. Under ordinary conditions of storage and use, these preparations can also contain a preservative to prevent the growth of microorganisms.

The pharmaceutical forms suitable for injectable use include sterile aqueous solutions and dispersions, and sterile powders for the extemporaneous preparation of sterile injectable solutions and dispersions. The preparation should be prepared under sterile conditions and be fluid to the extent that easy syringability exists. It should be sufficiently stable under the conditions of manufacture and storage and preserved against the growth of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and suitable oils.

Additionally, the present invention relates to use of a compound of any of (1)-(44) in the manufacture of a medicament for use in treating diseases or disorders associated with abnormal bone and mineral homeostasis, including but not limited to hypoparathyroidism, osteosarcoma, chondrosarcoma, periodontal disease, bone fracture healing, osteoarthritis, Paget's disease, osteopenia, glucocorticoid-induced osteoporosis, osteomalacia, osteoporosis, metastatic bone disease, abnormally increased bone turnover or joint replacement.

The present invention relates to the use of a compound of any of (1)-(44) in therapy, for example, in the treatment of diseases or disorders associated with abnormal bone and mineral homeostasis, including but not limited to hypoparathyroidism, osteosarcoma, chondrosarcoma, periodontal disease, bone fracture healing, osteoarthritis, Paget's disease, osteopenia, glucocorticoid-induced osteoporosis, osteomalacia, osteoporosis, metastatic bone disease, abnormally increased bone turnover or joint replacement.

The present invention further relates to a method for the treatment of diseases or disorders associated with abnormal bone and mineral homeostasis, including but not limited to hypoparathyroidism, osteosarcoma, chondrosarcoma, periodontal disease, bone fracture healing, osteoarthritis, Paget's disease, osteopenia, glucocorticoid-induced osteoporosis, osteomalacia, osteoporosis, metastatic bone disease, abnormally increased bone turnover or joint replacement comprising administering to an individual a pharmaceutical composition comprising a compound of any of (1)-(44).

Another embodiment of the present invention includes a method of treating a condition selected from: (1) hypoparathyroidism, (2) osteosarcoma, (3) chondrosarcoma, (4) periodontal disease, (5) bone fracture healing, (6) osteoarthritis, (7) Paget's disease, (8) osteopenia, (9) glucocorticoid-induced osteoporosis, (10) osteomalacia, (11) osteoporosis, (12) metastatic bone disease, (13) abnormally increased bone turnover or (14) joint replacement in a mammalian patient in need of such treatment, comprising administering to the patient a compound of any of (1)-(44) in an amount that is effective to treat said condition.

For dosing purposes, any suitable route of administration may be employed for providing a mammal, especially a human, with an effective amount of a compound of the present invention. Dosage forms may include tablets, troches, dispersions, suspensions, solutions, capsules, creams, ointments, aerosols, and the like. Most preferably, compounds of any of (1)-(44) are administered orally. The effective dosage of active ingredient employed may vary depending on the particular compound employed, the mode of administration, the condition being treated and the severity of the condition being treated. Such dosage may be ascertained readily by a person skilled in the art.

When treating or controlling osteoporosis or other diseases for which compounds of any of (1)-(44) are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 350 milligrams. For a particularly potent compound, the dosage for an adult human may be as low as 0.1 mg. The dosage regimen may be adjusted within this range or even outside of this range to provide the optimal therapeutic response. Oral administration will usually be carried out using tablets or capsules. Examples of doses in tablets and capsules are 0.1 mg, 0.25 mg, 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 12 mg, 15 mg, 20 mg, 25 mg, 50 mg, 100 mg, 200 mg, 350 mg, 500 mg, 700 mg, 750 mg, 800 mg and 1000 mg. Other oral forms may also have the same or similar dosages.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e., causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

The term “bone resorption,” as used herein, refers to the process by which osteoclasts degrade bone.

As discussed supra, compounds of the present invention may be used in combination with other drugs that may also be useful in the treatment or amelioration of the individual diseases and conditions described herein. Such other drugs may be administered by a route and in an amount commonly used therefore, contemporaneously or sequentially with a compound of any of (1)-(44). In the treatment of patients who have abnormal bone and mineral homeostasis, more than one drug can be administered. The compounds of this invention may generally be administered to a patient who is already taking one or more other drugs for these conditions.

When a compound of any of (1)-(44) is used contemporaneously with one or more other drugs, a pharmaceutical composition in unit dosage form containing such other drugs and the compound of any of (1)-(44) is preferred. However, the combination therapy also includes therapies in which a compound of any of (1)-(44) and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of any of (1)-(44).

If formulated as a fixed dose, such combination products employ the compounds of this invention within the dosage range described herein and the other pharmaceutically active agent(s) within its approved dosage range. Compounds of the instant invention may alternatively be used sequentially with known pharmaceutically acceptable agent(s) when a combination formulation is inappropriate.

Yet another embodiment of the present invention include a method of treating a condition selected from: (1) hypoparathyroidism, (2) osteosarcoma, (3) chondrosarcoma, (4) periodontal disease, (5) fracture healing, (6) osteoarthritis, (7) Paget's disease, (8) osteopenia, (9) glucocorticoid-induced osteoporosis, (10) osteomalacia, (11) osteoporosis, (12) metastatic bone disease, (13) abnormally increased bone turnover or (14) joint replacement in a mammalian patient in need of such treatment, comprising administering to the patient a compound of any of (1)-(44), and a compound useful in treating or preventing osteoporosis or other bone disorders.

A person of ordinary skill in the art would be able to discern which combinations of agents would be useful based on the particular characteristics of the drugs and the disease involved. Such agents include the following: an organic bisphosphonate; a selective estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; Vitamin D; a synthetic Vitamin D analogue; a Nonsteroidal anti-inflammatory drug; a selective cyclooxygenase-2 inhibitor; an inhibitor of interleukin-1 beta; a LOX/COX inhibitor; a cathepsin K inhibitor; and the pharmaceutically acceptable salts and mixtures thereof. A preferred combination is a compound of the present invention and an organic bisphosphonate. Another preferred combination is a compound of the present invention and a selective estrogen receptor modulator. Another preferred combination is a compound of the present invention and an androgen receptor modulator. Another preferred combination is a compound of the present invention and an osteoblast anabolic agent. Another preferred combination is a compound of the present invention and a cathepsin K inhibitor.

“Organic bisphosphonate” includes, but is not limited to, compounds of the chemical formula

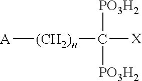

wherein n is an integer from 0 to 7 and wherein A and X are independently selected from the group consisting of H, OH, halogen, NH₂, SH, phenyl, C₁-C₃₀ alkyl, C₃-C₃₀ branched or cycloalkyl, bicyclic ring structure containing two or three N, C₁-C₃₀ substituted alkyl, C₁-C₁₀ alkyl substituted NH₂, C₃-C₁₀ branched or cycloalkyl substituted NH₂, C₁-C₁₀ dialkyl substituted NH₂, C₁-C₁₀ alkoxy, C₁-C₁₀ alkyl substituted thio, thiophenyl, halophenylthio, C₁-C₁₀ alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C₃-C₁₀ ring.

In the foregoing chemical formula, the alkyl groups can be straight, branched, or cyclic, provided sufficient atoms are selected for the chemical formula. The C₁-C₃₀ substituted alkyl can include a wide variety of substituents, nonlimiting examples which include those selected from the group consisting of phenyl, pyridyl, furanyl, pyrrolidinyl, imidazonyl, NH₂, C₁-C₁₀ alkyl or dialkyl substituted NH₂, OH, SH, and C₁-C₁₀ alkoxy.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C₁-C₁₀-alkyl-substituted ammonium. In particular embodiments, the salts are those selected from the group consisting of sodium, potassium, calcium, magnesium, and ammonium salts. In specific embodiments, the salts are sodium salts. Non-limiting examples of derivatives include those selected from the group consisting of esters, hydrates, and amides.

It should be noted that the terms “bisphosphonate” and “bisphosphonates”, as used herein are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein.

Non-limiting examples of bisphosphonates useful herein include the following:

Alendronate, which is also known as alendronic acid, 4-amino-1-hydroxybutylidene-1,1-bisphosphonic acid, alendronate sodium or alendronate monosodium trihydrate, 4-amino-1-hydroxybutylidene-1,1-bisphosphonic acid monosodium trihydrate.

Alendronate is described in U.S. Pat. No. 4,922,007, to Kieczykowski et al., issued May 1, 1990; U.S. Pat. No. 5,019,651, to Kieczykowski et al., issued May 28, 1991; U.S. Pat. No. 5,510,517, to Dauer et al., issued Apr. 23, 1996; U.S. Pat. No. 5,648,491, to Dauer et al., issued Jul. 15, 1997, all of which are incorporated by reference herein in their entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

1-hydroxy-2-imidazo-(1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zoledronate).

Nonlimiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

It is recognized that mixtures of two or more of the bisphosphonate actives can be utilized.

The precise dosage of the organic bisphosphonate will vary with the dosing schedule, the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight. For alendronate monosodium trihydrate, common human doses which are administered are generally in the range of about 2 mg/day to about 40 mg/day, preferably about 5 mg/day to about 40 mg/day. In the U.S., approved dosages for alendronate monosodium trihydrate are 5 mg/day for preventing osteoporosis, 10 mg/day for treating osteoporosis, and 40 mg/day for treating Paget's disease.

In alternative dosing regimens, the bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of 35 mg/week or 70 mg/week.

“Selective estrogen receptor modulators” refers to compounds which interfere or inhibit the binding of estrogen to the receptor, regardless of mechanism. Examples of estrogen receptor modulators include, but are not limited to, estrogen, progestogen, estradiol, droloxifene, raloxifene, lasofoxifene, TSE-424, tamoxifen, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

An “estrogen receptor beta modulator” is a compound that selectively agonizes or antagonizes estrogen receptor beta (ERβ Agonizing ERβ increases transcription of the tryptophan hydroxylase gene (TPH, the key enzyme in serotonin synthesis) via an ERβ mediated event. Examples of estrogen receptor beta agonists can be found in PCT International publication WO 01/82923, which published on Nov. 8, 2001, and WO 02/41835, which published on May 20, 2002, both of which are hereby incorporated by reference in their entirety.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“An inhibitor of osteoclast proton ATPase” refers to an inhibitor of the proton ATPase, which is found on the apical membrane of the osteoclast, and has been reported to play a significant role in the bone resorption process. This proton pump represents an attractive target for the design of inhibitors of bone resorption which are potentially useful for the treatment and prevention of osteoporosis and related metabolic diseases. See C. Farina et al., “Selective inhibitors of the osteoclast vacuolar proton ATPase as novel bone antiresorptive agents,” DDT, 4: 163-172 (1999)), which is hereby incorporated by reference in its entirety.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

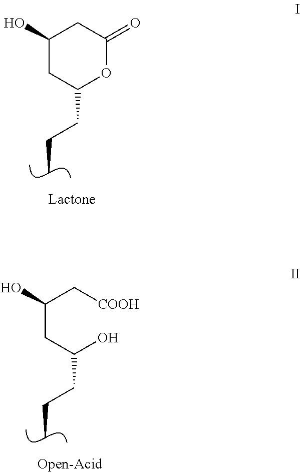

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

As used above, “integrin receptor antagonists” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins. H. N. Lode and coworkers in PNAS USA 96: 1591-1596 (1999) have observed synergistic effects between an antiangiogenic αᵥ integrin antagonist and a tumor-specific antibody-cytokine (interleukin-2) fusion protein in the eradication of spontaneous tumor metastases. Their results suggested this combination as having potential for the treatment of cancer and metastatic tumor growth. αᵥβ₃ integrin receptor antagonists inhibit bone resorption through a new mechanism distinct from that of all currently available drugs. Integrins are heterodimeric transmembrane adhesion receptors that mediate cell-cell and cell-matrix interactions. The α and β integrin subunits interact non-covalently and bind extracellular matrix ligands in a divalent cation-dependent manner. The most abundant integrin on osteoclasts is αᵥβ₃ (>10⁷/osteoclast), which appears to play a rate-limiting role in cytoskeletal organization important for cell migration and polarization. The αᵥβ₃ antagonizing effect is selected from inhibition of bone resorption, inhibition of restenosis, inhibition of macular degeneration, inhibition of arthritis, and inhibition of cancer and metastatic growth.

“An osteoblast anabolic agent” refers to agents that build bone, such as PTH. The intermittent administration of parathyroid hormone (PTH) or its amino-terminal fragments and analogues have been shown to prevent, arrest, partially reverse bone loss and stimulate bone formation in animals and humans. For a discussion refer to D. W. Dempster et al., “Anabolic actions of parathyroid hormone on bone,” Endocr Rev 14: 690-709 (1993). Studies have demonstrated the clinical benefits of parathyroid hormone in stimulating bone formation and thereby increasing bone mass and strength. Results were reported by R M Neer et al., in New Eng J Med 344 1434-1441 (2001).

In addition, parathyroid hormone-related protein fragments or analogues, such as PTHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)] and may also have potential as anabolic agents for treating osteoporosis.

“Vitamin D” includes, but is not limited to, vitamin D₃ (cholecalciferol) and vitamin D₂ (ergocalciferol), which are naturally occurring, biologically inactive precursors of the hydroxylated biologically active metabolites of vitamin D: 1α-hydroxy vitamin D; 25-hydroxy vitamin D, and 1α,25-dihydroxy vitamin D. Vitamin D₂ and vitamin D₃ have the same biological efficacy in humans. When either vitamin D₂ or D₃ enters the circulation, it is hydroxylated by cytochrome P₄₅₀-vitamin D-25-hydroxylase to give 25-hydroxy vitamin D. The 25-hydroxy vitamin D metabolite is biologically inert and is further hydroxylated in the kidney by cytochrome P450-monooxygenase, 25 (OH) D-1α-hydroxylase to give 1,25-dihydroxy vitamin D. When serum calcium decreases, there is an increase in the production of parathyroid hormone (PTH), which regulates calcium homeostasis and increases plasma calcium levels by increasing the conversion of 25-hydroxy vitamin D to 1,25-dihydroxy vitamin D.

1,25-dihydroxy vitamin D is thought to be responsible for the effects of vitamin D on calcium and bone metabolism. The 1,25-dihydroxy metabolite is the active hormone required to maintain calcium absorption and skeletal integrity. Calcium homeostasis is maintained by 1,25-dihydroxy vitamin D by inducing monocytic stem cells to differentiate into osteoclasts and by maintaining calcium in the normal range, which results in bone mineralization by the deposition of calcium hydroxyapatite onto the bone surface, see Holick, M F, Vitamin D photobiology, metabolism, and clinical applications, In: DeGroot L, Besser H, Burger H G, eg al., eds. Endocrinology, 3^{rd} ed., 990-1013 (1995). However, elevated levels of 1α,25-dihydroxy vitamin D₃ can result in an increase of calcium concentration in the blood and in the abnormal control of calcium concentration by bone metabolism, resulting in hypercalcemia. 1a,25-dihydroxy vitamin D₃ also indirectly regulates osteoclastic activity in bone metabolism and elevated levels may be expected to increase excessive bone resorption in osteoporosis.

“Synthetic vitamin D analogues” includes non-naturally occurring compounds that act like vitamin D.

“Nonsteroidal anti-inflammatory drugs” or NSAIDs, inhibit the metabolism of arachidonic acid to proinflammatory prostaglandins via cyclooxygenase (COX)-1 and COX-2. Nonlimiting examples of NSAIDs include: aspirin, ibuprofen, naproxen, diclofenac, etodolac, fenoporfen, flubiprofen, indomethacin, ketoprofen, ketorolac, meloxicam, nabumetone, oxaprozin, piroxicam, sulindac, tolmetin, diflunisal, meclofenamate and phenylbutazone.

A “selective cyclooxygenase-2 inhibitor,” or COX-2 inhibitor, refers to a type of nonsteroidal anti-inflammatory drug (NSAID), that inhibit the COX-2 coenzyme, which contributes to pain and inflammation in the body. Nonlimiting examples of COX-2 inhibitors include: celecoxib, etoricoxib, parecoxib, rofecoxib, valdecoxib and lumiracoxib.

An “inhibitor of interleukin-1 beta” or IL-1β refers to in inhibitors of IL-1, which is a soluble factor produced by monocytes, macrophages, and other cells which activates T-lymphocytes and potentiates their response to mitogens or antigens. Nonlimiting examples of IL-1B inhibitors include diacerein and rhein.

A “LOX/COX inhibitor” refers to an inhibitor or all three of the major enzymes involved in arachidonic acid pathway—namely, 5-LOX, COX-1 and COX-2. A nonlimiting example of a LOX/COX inhibitor is licofelone.

A “cathepsin K inhibitor” refers to an inhibitor of cathepsin K, an enzyme involved in bone resorption. Nonlimiting examples of cathepsin K inhibitors include Novartis's AAE-581, balicatib, GlaxoSmithKline's SB-462795 and odanacatib.

These and other aspects of the invention will be apparent from the teachings contained herein.

### EXAMPLES

The compounds of the invention can be prepared using the synthetic schemes and experimental procedures described herein as well as any of several alternate methods which will be apparent to a chemist skilled in the art; see, e.g., Synthetic Communications, 16(13), 1635-1640 (1986). The following abbreviations may be used in the synthetic schemes and experimental procedures: BCA is bicinchoninic acid; Bn is benzyl; BSA is bovine serum albumin; DCM is dichloromethane; DMF is N,N-dimethylformamide; DMSO is dimethyl sulfoxide; EDTA is ethylenediaminetetraacetic acid; EGTA is ethylene glycol tetraacetic acid; ELISA is enzyme-linked immunosorbent assay; HBSS is Hank's Balanced Salt Solution; hr is hour; HCl is hydrochloride acid; HEPES is 4-(2-Hydroxyethyl)piperazine-1-ethanesulfonic acid; HPLC is high performance liquid chromatography; min is minute; prep-HPLC is preparative high performance liquid chromatography; N/A is not available; PBS is phosphate-buffered saline; PEI is polyethylenimine; prep-TLC is preparative thin layer chromatography; PTH is parathyroid hormone; RT is room temperature; sec is second; TBDMSCl is tert-butyldimethylchlorosilane; THF is tetrahydrofuran; TLC is thin layer chromatography; Tf₂O is triflic anhydride.

### Example 1

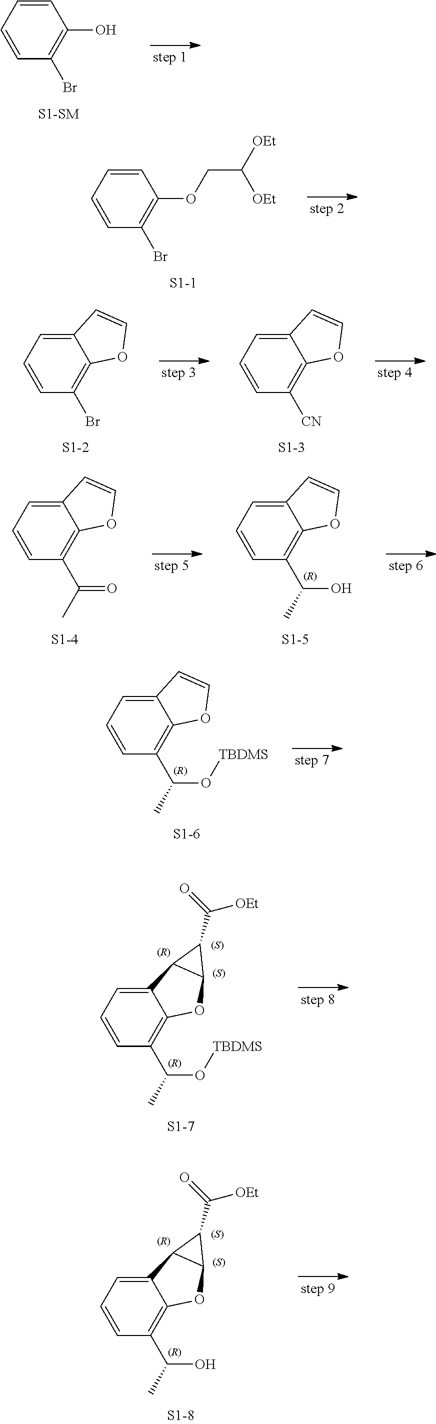

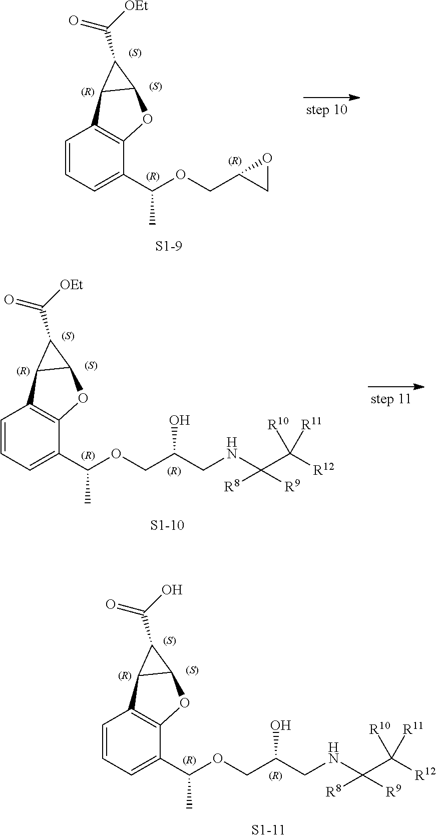

### Experimental Procedure

### Step 1

2-Bromophenol (S1-SM, 181.2 g, 1.04 mol) was dissolved in anhydrous DMF (300 mL) and the solution was added to a suspension of sodium hydride (46 g, 1.12 mol) in anhydrous DMF (900 mL) at 0° C. The mixture was stirred for an additional 30 mins and 2-bromo-1,1-diethoxyethane (307.2 g, 1.56 mol) was added. Then the mixture was refluxed at 130° C. for 2 hrs. After the mixture was cooled, it was poured into water. The mixture was concentrated and the residue was extracted with ethyl acetate. The organic layer was washed by brine, separated, dried over anhydrous sodium sulfate, filtered and concentrated to afford S1-1.

### Step 2

Polyphosphoric acid (480 g) and chlorobenzene (800 mL) were combined and the mixture was heated to reflux. To the refluxing mixture was added dropwise a solution of S1-1 (224 g, 0.744 mol) in chlorobenzene (160 mL) over 30 mins. The reaction mixture was refluxed for 2 hrs, then it was cooled to room temperature. 1N NaOH (800 mL) was added and the mixture was stirred at room temperature overnight. The reaction mixture was extracted with ethyl acetate. The organic layer was washed with brine, separated, dried over anhydrous sodium sulfate, filtered, and concentrated to afford the product which was purified by silica gel to give S1-2.

### Step 3

A mixture of S1-2 (13 g, 0.07 mol), CuCN (18.1 g, 0.2 mol) and CuI (25.3 g, 0.13 mol) in DMF (120 mL) was stirred at 150° C. for 4 hrs. Cooled, the mixture was diluted with ethyl acetate and washed with brine. The organic layer was separated, dried over anhydrous sodium sulfate, filtered and concentrated to provide a residue, which was purified by column chromatography to afford S1-3.

### Step 4

To a solution of S1-3 (4 g, 0.03 mol) in toluene (30 mL) was added dropwise methylmagnesium bromide (28 mL, 3.0 M, 0.08 mol) at room temperature under N₂. The mixture was stirred at 60° C. for 1 hr. NH₄Cl (aq.) was added and the mixture was acidified with 1N HCl. The resulting mixture was refluxed for 1 hr, and it was extracted with ethyl acetate. The organic layer was washed with saturated aqueous NaHCO₃ solution and brine, separated, dried over anhydrous sodium sulfate, filtered and concentrated to provide a residue, which was purified by column chromatography to afford S1-4.

### Step 5

(S)-diphenyl prolinol (500 mg) were added to anhydrous THF, then B(OCH₃)₃ (0.43 mL) were added to the solution at 0° C. ˜−10° C. under N₂. The mixture was stirred overnight. A solution of BH₃—S(CH₃)₂ in THF was added to it. Then a solution of S1-4 in THF was added by syringe pump at 0° C. ˜−10° C. over 5 hr. TLC indicated the completion of the reaction. The reaction was quenched with HCl (2N), and the mixture was extracted with ethyl acetate. The organic layer was washed with saturated aqueous NaHCO₃ solution and brine, separated, dried over anhydrous sodium sulfate, filtered and concentrated to provide a residue, which was purified by column chromatography to afford S1-5.

### Step 6

To a solution of S1-5 (3.0 g, 0.018 mol) in DMF (50 mL) was added imidazole (3.3 g, 0.046 mol), followed by tert-butyldimethylchlorosilane (TBDMSCl, 3.3 g, 0.022 mol) at 0° C. The mixture was stirred at room temperature for 2 hrs. Then the reaction mixture was diluted with ethyl acetate and washed with brine. The organic layer was separated, dried over anhydrous sodium sulfate, filtered and concentrated to provide a residue, which was purified by column chromatography to afford S1-6.

### Step 7

Copper (I) triflate (2:1 complex with toluene, 95 mg, 0.36 mmol) and (R,R)-(+)-2,2-Isopropylidenebis(4-tert-butyl-2-oxazoline) (0.135 g, 0.46 mmol, DL Chiral Chemicals) were stirred in DCM (20 mL) at room temperature under N₂ atmosphere for 1.5 hrs. A drop of ethyl diazoethanoate was added to this deep green solution. The color temporarily faded to brown and gas evolving was observed. A solution of S1-6 (4.5 g, 0.016 mol) in DCM (80 mL) was added, followed by a slow addition of a solution of ethyl diazoethanoate (9.5 mL, 0.09 mol) in DCM (40 mL) during a period of 16 hrs using a syringe pump. The reaction was stirred at room temperature for 2 hrs after the addition. The mixture was concentrated and the residue was purified by column chromatography to afford S1-7, which was directly used in the next step.

### Step 8

Tetrabutylammonium fluoride (16 mL, 1 M in THF, 0.016 mol) was added dropwise to a solution of S1-7 (4.0 g, 0.011 mol) in THF (50 mL) at 0° C. The reaction was stirred at room temperature for 6 hrs. The mixture was filtered and washed with ethyl acetate. The filtrate was washed with brine. The organic layer was separated, dried over anhydrous sodium sulfate, filtered and concentrated to provide a residue, which was purified by column chromatography to afford S1-8.

### Step 9

To a mixture of S1-8 (1.2 g, 0.005 mol) and (R)-oxiran-2-ylmethyl 3-nitrobenzenesulfonate (1.4 g, 0.005 mol) in DMF (10 mL) was added NaH (0.19 g, 0.005 mol) at 0° C. The reaction was stirred at room temperature for 2 days. The mixture was diluted with ethyl acetate and washed with brine. The organic layer was separated, dried over anhydrous sodium sulfate, filtered and concentrated to provide a residue, which was purified by column chromatography to afford S1-9.

### Step 10

To a solution of S1-9 (100 mg, 0.3 mmol) in DMF (2 mL) was added amine (80 mg, 0.4 mmol) at room temperature. The solution was stirred overnight. Then the reaction solution was diluted with ethyl acetate and washed with brine. The organic layer was separated, dried over anhydrous sodium sulfate, filtered and concentrated to provide a residue, which was purified by prep-TLC to afford S1-10.

### Step 11

2N aq. NaOH (0.5 mL) was added to S1-10 (0.207 mmol) in MeOH (1.0 mL) and THF (1.0 mL). The solution was stirred at 60° C. for 0.5 hrs. Then MeOH and THF were removed under reduced pressure. 1N aq. HCl was added to the reaction in water (10 mL) to make PH=7. The precipitate was collected and purified by prep-HPLC to afford the product S1-11.

The following compounds in Table 1 were made according to EXAMPLE 1.

TABLE 1

| | | FLIPR ASSAY | |
|---|---|---|---|
| Example | Structure | IC₅₀ (nM) | Analysis Data |
| 1-1 | 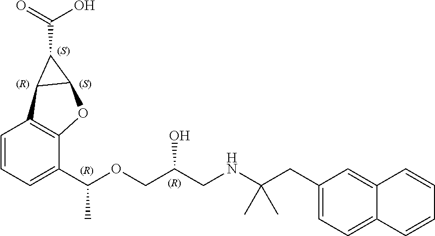 | 26 | ¹H NMR (301 MHz, CD₃OD) δ 7.82 (dd, J = 15.8, 13.5 Hz, 4H), 7.55-7.43 (m, 2H), 7.39 (dd, J = 6.2, 4.0 Hz, 2H), 7.17 (t, J = 6.6 Hz, 1H), 6.96 (t, J = 7.6 Hz, 1H), 5.12 (d, J = 5.4 Hz, 1H), 4.67 (q, J = 6.5 Hz, 1H), 4.01 (d, J = 5.1 Hz, 1H), 3.57-3.43 (m, 1H), 3.34 (d, J = 5.2 Hz, 3H), 3.19 (d, J = 13.4 Hz, 2H), 3.06 (dt, J = 21.5, 9.4 Hz, 1H), 1.38 (d, J = 5.2 Hz, 9H), 1.12 (dd, J = 8.0, 2.2 Hz, 1H). LC- MS (ESI) 476.2 [M + 1]. HPLC (220 nm): 96.2%. |
| 1-2 | 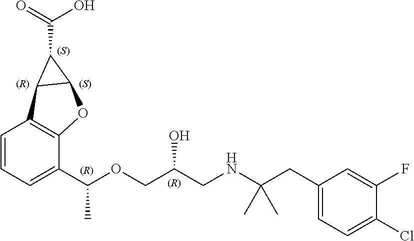 | 148 | ¹H NMR (301 MHz, CD₃OD) δ 7.42-7.22 (m, 2H), 7.11 (dd, J = 8.5, 6.4 Hz, 2H), 7.03-6.77 (m, 2H), 5.10-4.93 (m, 1H), 4.59 (q, J = 6.3 Hz, 1H), 3.88 (d, J = 5.0 Hz, 1H), 3.37 (dd, J = 9.8, 4.9 Hz, 1H), 3.21 (dd, J = 6.0, 4.4 Hz, 3H), 3.14-3.08 (m, 3H), 1.42-1.28 (m, 3H), 1.21 (s, 6H), 1.00 (d, J = 3.0 Hz, 1H). LC-MS (ESI) 478.1 [M + 1]. HPLC (220 nm): 98.96%. |
| 1-3 | 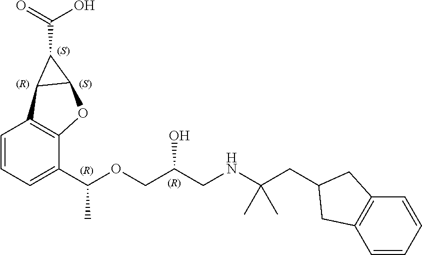 | 40 | ¹H NMR (301 MHz, CD₃OD) δ 7.38 (d, J = 7.4 Hz, 1H), 7.21 (t, J = 7.6 Hz, 1H), 7.12 (td, J = 7.9, 3.7 Hz, 4H), 6.96 (t, J = 7.5 Hz, 1H), 5.12 (d, J = 5.3 Hz, 1H), 4.76-4.59 (m, 1H), 3.97 (d, J = 5.1 Hz, 1H), 3.49 (dd, J = 9.8, 4.5 Hz, 1H), 3.30-3.04 (m, 5H), 3.03-2.80 (m, 1H), 2.75-2.42 (m, 3H), 1.98 (d, J = 6.0 Hz, 2H), 1.46 (d, J = 7.0 Hz, 9H), 1.20-0.94 (m, 1H). LC-MS (ESI) 466.2 [M + 1]. HPLC (220 nm): 96.35%. |
| 1-4 | 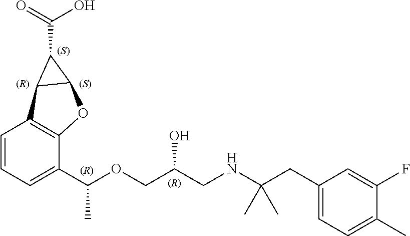 | 78 | ¹H NMR (MeOD, 300 MHz) δ 7.45-7.32 (m, 1H), 7.30- 7.15 (m, 2H), 7.10-6.90 (m, 3H), 5.20-5.12 (m, 1H), 4.70- 4.62 (m, 1H), 4.08-3.92 (m, 1H), 3.50-3.38 (m, 1H), 3.15- 2.80 (m, 3H), 2.25 (s, 3H), 1.48-1.40 (m, 3H), 1.35 (s, 6H), 1.15 (s, 1H). LC-MS (ESI): 457.2 [M + 1]. HPLC (220 nm): 98.2%. |
| 1-5 | 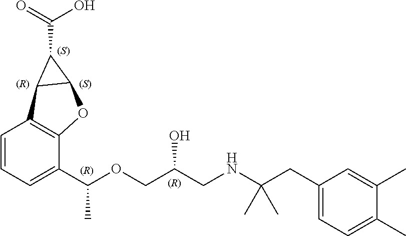 | 230 | ¹H NMR (MeOD, 300 MHz) δ 8.10-7.90 (m, 1H), 7.50- 7.30 (m, 1H), 7.20-7.15 (m, 1H), 7.12-6.95 (m, 1H), 6.90- 6.80 (m, 2H), 5.20-5.12 (m, 1H), 4.70-4.62 (m, 1H), 4.08- 3.92 (m, 1H), 3.60-3.40 (m, 1H), 3.25-2.90 (m, 3H), 2.85- 2.70 (m, 1H), 2.68-2.40 (m, 1H), 2.30-2.20 (m, 6H), 1.48- 1.32 (m, 3H), 1.25 (s, 6H), 1.19-1.00 (m, 1H). LC-MS (ESI): 454.2 [M + 1]. HPLC (220 nm): 92.3%. |
| 1-6 | 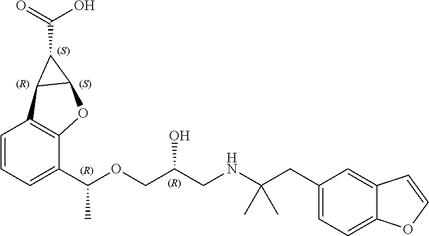 | 49 | ¹H NMR (MeOD, 300 MHz) δ 7.66 (s, 1H), 7.45-7.32 (m, 1H), 7.30-7.25 (m, 1H), 7.12- 7.01 (m, 2H), 6.90-6.82 (m, 1H), 6.73 (s, 1H), 5.08-4.96 (m, 1H), 4.65-4.50 (m, 1H), 3.90-3.85 (m, 1H), 3.40-3.30 (m, 2H), 3.20-3.15 (m, 1H), 3.00-2.85 (m, 4H), 1.30-1.25 (m, 3H), 1.22 (s, 6H), 1.00 (s, 1H). LC-MS (ESI) 466.2 [M + 1]. HPLC (220 nm): 95.11%. |
| 1-7 | 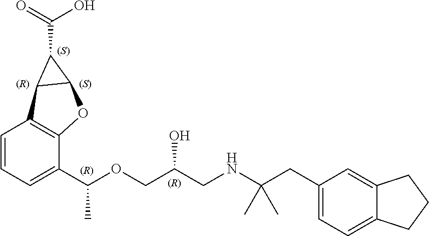 | 110 | ¹HNMR (MeOD, 300 MHz) δ 7.45-7.20 (m, 1H), 7.15-7.05 (m, 2H), 7.05-6.90 (s, 1H), 6.85-6.75 (m, 2H), 5.15-4.90 (m, 1H), 4.60-4.50 (m, 1H), 3.85 (s, 1H), 3.65-3.55 (m, 2H), 3.40-3.30 (m, 1H), 3.20- 3.15 (m, 1H), 3.00-2.60 (m, 7H), 2.05-1.85 (m, 2H), 1.80- 1.65 (m, 2H), 1.35 (d, J = 7.0 Hz, 3H), 1.21 (s, 6H), 1.0 (s, 1H). LC-MS (ESI): 466.3 [M + 1]. HPLC (220 nm): 88.60%. |
| 1-8 | 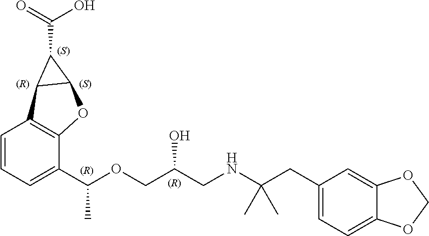 | 320 | ¹H NMR (MeOD, 300 MHz) δ 7.40-7.35 (m, 1H), 7.25- 7.15 (m, 1H), 7.00-6.90 (m, 1H), 6.82-6.70 (m, 3H), 5.94 (s, 2H), 5.15-5.08 (m, 1H), 4.75-4.62 (m, 1H), 4.00-3.82 (m, 1H), 3.52-3.40 (m, 2H), 3.30-3.20 (m, 2H), 3.10-2.85 (m, 3H), 1.50-1.40 (m, 3H), 1.30 (s, 6H), 0.91 (s, 1H). LC-MS (ESI) 470.1 [M + 1]. HPLC (220 nm): 98.50%. |
| 1-9 | 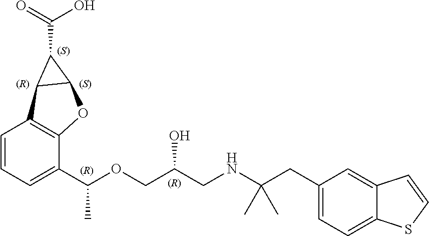 | 27 | ¹H NMR (MeOD, 300 MHz) δ 7.90-7.80 (m, 1H), 7.75 (s, 1H), 7.68-7.55 (m, 1H), 7.48- 7.30 (m, 2H), 7.28-6.60 (m, 3H), 5.17-5.0 (m, 1H), 4.70- 4.65 (m, 1H), 4.10-3.82 (m, 2H), 3.68-3.60 (m, 1H), 3.55- 3.46 (m, 1H), 3.30-3.22 (m, 1H), 3.20-2.75 (m, 4H), 1.95- 1.80 (m, 2H), 1.40-1.32 (m, 9H), 1.17-1.10 (m, 1H). LC-MS (ESI): 482.2 [M + 1]. HPLC (220 nm): 97.46%. |
| 1-10 | 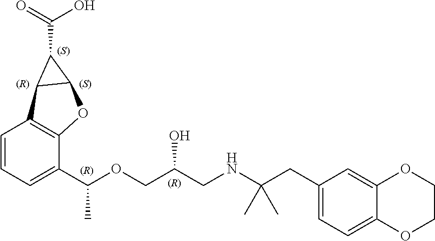 | 460 | ¹H NMR (MeOD, 300 MHz) δ 7.45-7.32 (m, 1H), 7.25- 7.18 (m, 1H), 7.05-6.90 (m, 1H), 6.80-6.73 (m, 1H), 6.70- 6.65 (m, 2H), 5.20-5.10 (m, 1H), 4.75-4.62 (m, 1H), 4.20 (s, 4H), 4.00-3.90 (m, 1H), 3.50-3.40 (m, 1H), 3.25-3.20 (m, 1H), 3.08-2.90 (m, 3H), 2.88-2.80 (m, 2H), 1.50-1.35 (d, J = 7.0 Hz, 3H), 1.25 (s, 6H), 1.18-1.10 (m, 1H). LC- MS (ESI) 484.2 [M + 1]. HPLC (220 nm): 99.04%. |
| 1-11 | 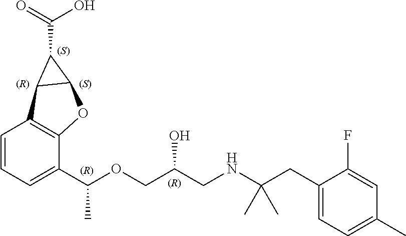 | 140 | δ 7.35 (MeOD, 300 MHz) (d, J = 7.3 Hz, 1H), 7.17 (t, J = 6.6 Hz, 2H), 7.05-6.89 (m, 3H), 5.06 (d, J = 5.3 Hz, 1H), 4.69 (q, J = 6.4 Hz, 1H), 4.10-3.93 (m, 1H), 3.51-3.43 (m, 1H), 3.34-3.31 (m, 1H), 3.28-3.17 (m, 2H), 3.09-2.91 (m, 3H), 2.34 (s, 3H), 1.43 (d, J = 6.4 Hz, 3H), 1.30 (s, 6H), 1.07-1.04 (m, 1H); LC-MS (ESI) 458.2 [M + 1]; HPLC (220 nm): 99.0%. |
| 1-12 | 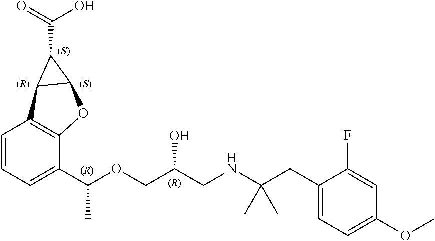 | 240 | ¹H NMR (MeOD, 300 MHz) δ 7.38 (dd, J = 7.4, 1.1 Hz, 1H), 7.26-7.09 (m, 2H), 6.96 (t, J = 7.5 Hz, 1H), 6.81-6.65 (m, 2H), 5.09 (d, J = 5.4 Hz, 1H), 4.69 (q, J = 6.2 Hz, 1H), 3.95 (dd, J = 16.1, 5.7 Hz, 1H), 3.80 (s, 3H), 3.58-3.41 (m, 1H), 3.35 (s, 1H), 3.30- 3.22 (m, 2H) 2.98 (dt, J = 19.0, 11.9 Hz, 3H), 1.44 (d, J = 6.5 Hz, 3H), 1.33 (d, J = 16.8 Hz, 6H), 1.08 (d, J = 2.3 Hz, 1H).. LC-MS (ESI): 474.2 [M + 1]. HPLC (220 nm): 95.06%. |
| 1-13 | 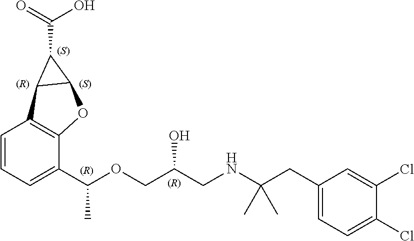 | 39 | ¹H NMR (MeOD, 300 MHz) δ 7.60-7.42 (m, 2H), 7.40- 7.30 (m, 2H), 7.28-7.15 (m, 2H), 7.08-6.95 (m, 1H), 5.20- 5.10 (m, 1H), 4.75-4.60 (m, 1H), 4.05-3.90 (m, 1H), 3.50- 3.40 (m, 1H), 3.15-2.95 (m, 3H), 1.48-1.40 (d, J = 7.0 Hz, 3H), 1.27 (s, 6H), 1.19-1.00 (m, 1H). LC-MS (ESI): 494.1 [M + 1]. HPLC (220 nm): 98.28%. |
| 1-14 | 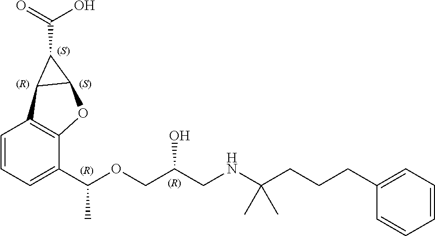 | 38 | ¹HNMR (MeOD, 300 MHz) δ 7.40-7.12 (m, 7H), 6.93- 6.68 (m, 1H), 5.12-5.00 (m, 1H), 4.70-4.60 (m, 1H), 3.95- 3.80 (m, 1H), 3.58-3.38 (m, 1H), 3.25-2.96 (m, 3H), 2.80- 2.60 (m, 3H), 1.70-1.58 (m, 4H), 1.50-1.40 (d, J = 5.3 Hz, 2H), 1.30-1.25 (m, 6H), 1.04 (s, 1H). LC-MS (ESI): 454.2 [M + 1]. HPLC (220 nm): 95.61%. |
| 1-15 | 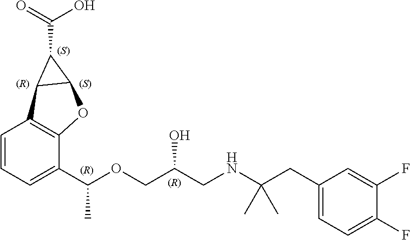 | 1266 | ¹H NMR (MeOD, 300 MHz) δ 7.30-7.25 (d, J = 6.5 Hz, 1H), 7.42-7.03 (m, 3H), 7.00-6.90 (m, 1H), 6.85-6.60 (m, 1H), 5.03-4.92 (d, J = 7.6 Hz, 1H), 4.68-4.56 (m, 1H), 3.90-3.80 (m, 1H), 3.70-3.62 (m, 2H), 3.47-3.32 (m, 1H), 3.20-3.12 (m, 2H), 3.00-2.86 (m, 3H), 1.92-1.70 (m, 2H), 1.42-1.30 (d, J = 5.2 Hz, 3H), 1.24 (s, 6H), 1.08-0.98 (m, 1H). LC- MS (ESI): 462.2 [M + 1]. HPLC (220 nm): 95.1%. |
| 1-16 | 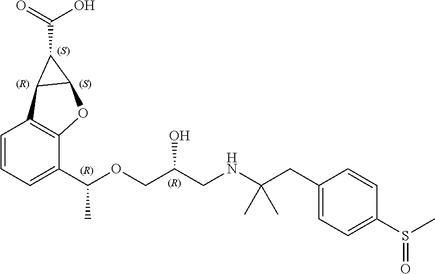 | >10000 | ¹H NMR (MeOD, 300 MHz) δ 7.62-7.55 (m, 2H), 7.46-7.38 (m, 2H), 7.30-7.22 (m, 1H), 7.20-7.13 (m, 1H), 6.90-6.82 (m, 1H), 5.08-4.95 (m, 1H), 4.70-4.52 (m, 1H), 4.00-3.83 (m, 1H), 3.50-3.30 (m, 1H), 3.28-3.20 (m, 1H), 3.08-2.86 (m, 3H), 2.70 (s, 3H), 1.42- 1.30 (d, J = 8.2 Hz, 2H), 1.24 (s, 6H), 1.08-1.00 (m, 1H). LC-MS (ESI): 488.2 [M +1]. HPLC (220 nm): 99.63%. |
| 1-17 | 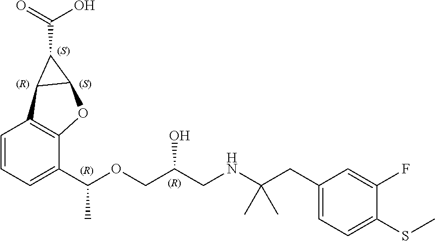 | 165 | ¹H NMR (MeOD, 300 MHz) δ 7.30-7.25 (d, J = 7.4 Hz, 1H), 7.21-7.18 (m,, 1H), 7.18- 7.10 (m, 1H), 7.0-6.86 (m, 3H), 5.12-4.97 (d, J = 5.3 Hz, 1H), 4.66-4.52 (m, 1H), 3.90- 3.75 (m, 1H), 3.48-3.30 (m, 1H), 3.30-2.75 (m, 4H), 2.40 (s, 3H), 1.38-1.30 (d, J = 6.0 Hz, 2H), 1.20 (s, 6H), 1.08- 0.94 (m, 1H). LC-MS (ESI): 490.2 [M + 1]. HPLC (220 nm): 100%. |
| 1-18 | 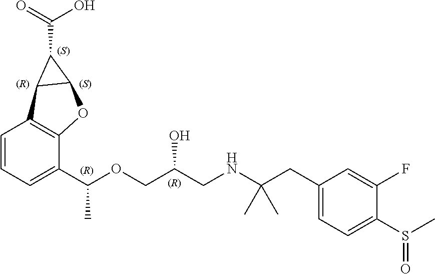 | >10000 | ¹H NMR (MeOD, 300 MHz) δ 7.72-7.60 (m, 1H), 7.38-7.20 (m, 2H), 7.18-7.06 (m, 2H), 6.92-6.80 (m, 1H), 5.08-4.95 (m, 1H), 4.70-4.52 (m, 1H), 4.00-3.83 (m, 1H), 3.46-3.30 (m, 1H), 3.10-2.80 (m, 3H), 2.78-2.70 (m, 3H), 1.37-1.30 (d, J = 8.2 Hz, 2H), 1.25 (s, 6H), 1.08-1.00 (m, 1H). LC- MS (ESI): 505.2 [M + 1]. HPLC (220 nm): 95.6%. |
| 1-19 | 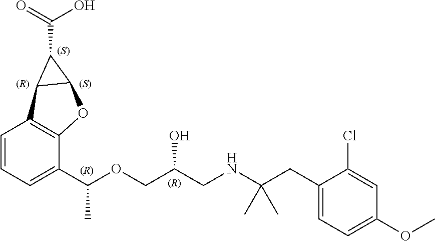 | 170 | ¹H NMR (MeOD, 300 MHz) δ 7.40-7.20 (m, 2H), 7.20-7.10 (m, 1H), 7.00-6.92 (m, 1H), 6.80-6.70 (m, 2H), 5.10-4.95 (m, 1H), 4.75-4.60 (m, 1H), 4.00-3.80 (m, 1H), 3.68 (s, 3H), 3.50-3.40 (m, 1H), 3.15- 2.90 (m, 4H), 2.85-2.80 (m, 1H), 1.50-1.40 (d, J = 6.5 Hz, 3H), 1.25 (s, 6H), 0.97 (s, 1H). LC-MS (ESI): 490.2 [M + 1]. HPLC (220 nm): 100%. |
| 1-20 | 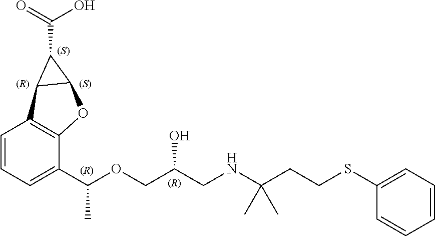 | 330 | ¹H NMR (MeOD, 300 MHz) δ 7.35-7.25 (m, 3H), 7.25-7.18 (m, 2H), 7.15-7.00 (m, 2H), 6.90-6.60 (m, 2H), 5.05-4.98 (m, 1H), 4.62-4.50 (m, 1H), 3.85-3.75 (m, 1H), 3.35-3.25 (m, 1H), 3.20-3.10 (m, 1H), 3.05-2.80 (m, 4H), 2.75-2.62 (m, 1H), 1.90-1.80 (m, 2H), 1.38-1.30 (d, J = 7.0 Hz, 3H), 1.28 (s, 6H), 1.00 (s, 1H). LC-MS (ESI) 472.2 [M + 1]. HPLC (220 nm): 95.42%. |
| 1-21 | 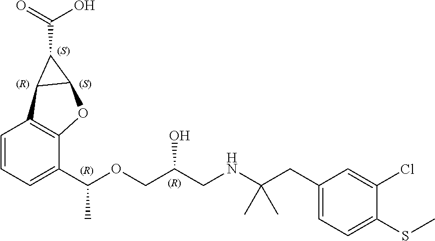 | 86 | ¹H NMR (MeOD, 300 MHz) δ 7.30-7.23 (m, 1H), 7.20-7.15 (m, 1H), 7.15-7.05 (m, 3H), 6.90-6.80 (m, 1H), 5.05-4.90 (d, J = 5.3 Hz, 1H), 4.65-4.52 (m, 1H), 3.90-3.78 (m, 1H), 3.40-3.35 (m, 1H), 3.18-3.10 (m, 1H), 3.00-2.80 (m, 3H), 2.35 (s, 3H), 1.40-1.30 (d, J = 8.5 Hz, 3H), 1.25 (s, 6H), 0.98 (s, 1H). LC-MS (ESI): 506.2 [M + 1]. HPLC (220 nm): 95.67% |
| 1-12 | 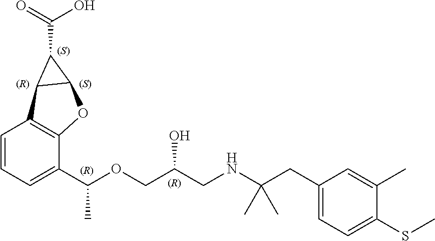 | 170 | ¹H NMR (MeOD, 300 MHz) δ 7.45-7.35 (m, 1H), 7.25- 7.18 (m, 2H), 7.15-7.00 (m, 2H), 7.00-6.90 (m, 1H), 5.16 (d, J = 5.2 Hz, 1H), 4.75-4.60 (m, 1H), 4.00-3.86 (m, 2H), 3.58-3.40 (m, 1H), 3.10-2.85 (m, 4H), 2.45 (s, 3H), 2.28 (s, 3H), 1.50-1.32 (d, J = 6.1 Hz, 3H), 1.25 (s, 6H), 1.08- 1.00 (m, 1H). LC-MS (ESI): 486.1 [M + 1]. HPLC (220 nm): 95.10%. |

### Example 2

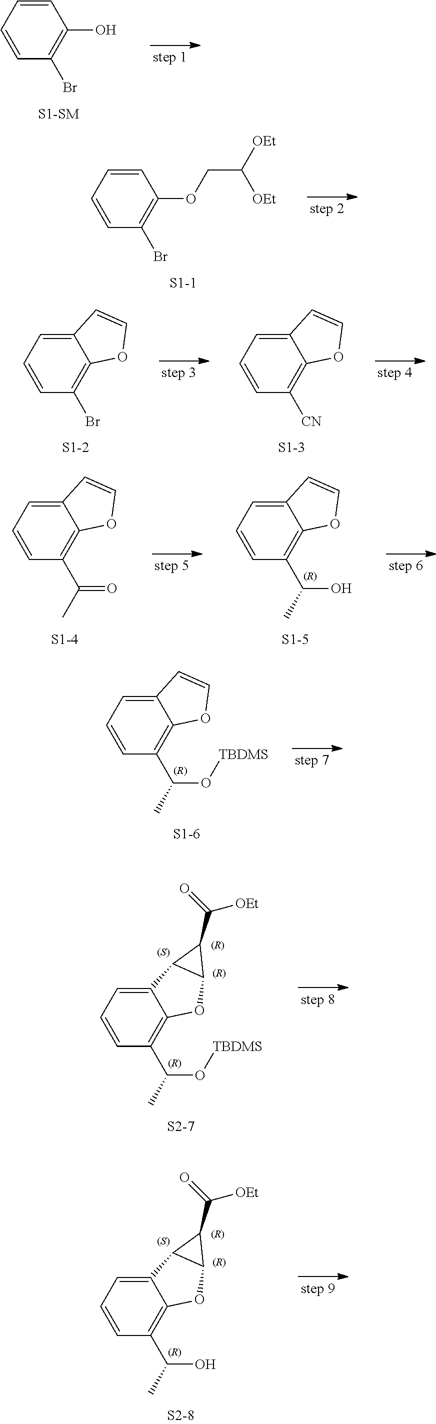

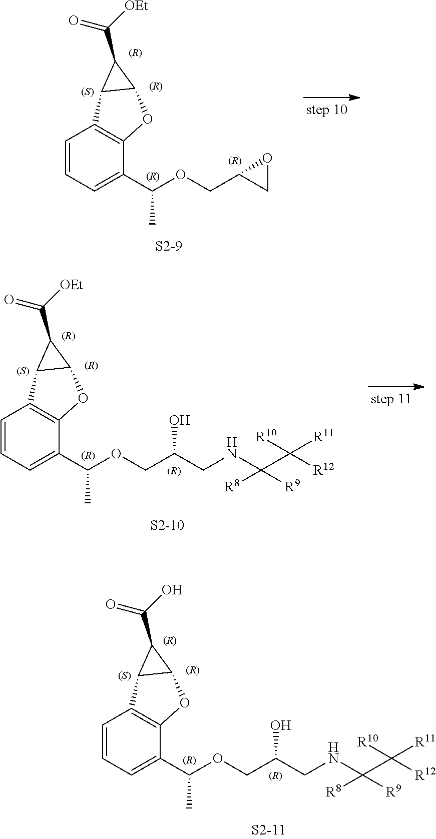

### Experimental Procedure

The experimental procedure in EXAMPLE 2 was the same as that in EXAMPLE 1 except (S,S)-(−)-2,2-Isopropylidenebis(4-tert-butyl-2-oxazoline) was used as the chiral ligand in Step 7.

The following compounds in Table 2 were made according to EXAMPLE 2.

TABLE 2

| | | FLIPR ASSAY | |
|---|---|---|---|
| Example | Structure | IC₅₀ (nM) | Analysis Data |
| 2-1 | 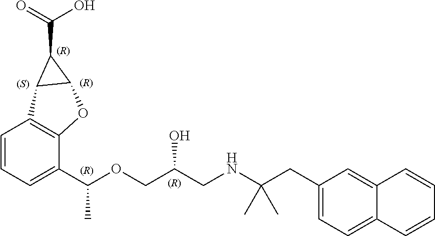 | 132 | ¹H NMR (301 MHz, CD₃OD) δ 8.47 (s, 1H), 7.86 (dd, J = 32.5, 24.6 Hz, 5H), 7.53-7.27 (m, 4H), 7.15-6.85 (m, 1H), 5.05 (s, 1H), 4.65 (d, J = 6.3 Hz, 1H), 4.05 (s, 1H), 3.63- 3.43 (m, 1H), 3.20 (s, 3H), 3.03-2.80 (m, 3H), 2.09 (dt, J = 13.2, 6.9 Hz, 1H), 1.37 (d, J = 7.0 Hz, 9H), 1.17 (d, J = 67.4 Hz, 1H). LC-MS (ESI) 476.2 [M + 1]. HPLC (220 nm): 98.4%. |
| 2-2 | 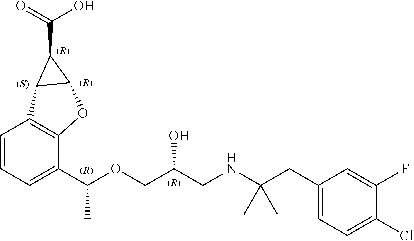 | 148 | ¹H NMR (301 MHz, CD₃OD) δ 7.45 (t, J = 8.0 Hz, 1H), 7.34 (d, J = 7.2 Hz, 1H), 7.27-7.06 (m, 3H), 6.93 (t, J = 7.5 Hz, 1H), 5.05 (s, 1H), 4.73-4.58 (m, 1H), 4.01 (s, 1H), 3.64- 3.39 (m, 1H), 3.20 (s, 1H), 2.97 (dd, J = 28.8, 20.0 Hz, 4H), 2.09 (dt, J = 14.0, 7.3 Hz, 1H), 1.44 (d, J = 6.4 Hz, 3H), 1.33 (s, 6H), 1.30 (s, 1H). LC- MS (ESI) 478.1 [M + 1]. HPLC (220 nm): 97.77%. |
| 2-3 | 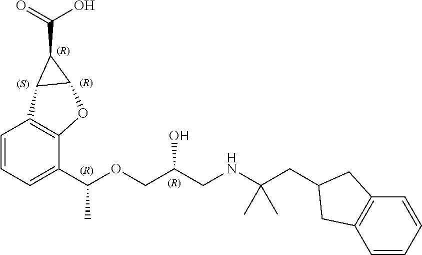 | 307 | ¹H NMR (301 MHz, CD₃OD) δ 7.38 (d, J = 7.4 Hz, 1H), 7.21 (t, J = 7.6 Hz, 3H), 7.12 (td, J = 7.9, 3.7 Hz, 2H), 6.96 (t, J = 7.5 Hz, 1H), 5.12 (d, J = 5.3 Hz, 1H), 4.76-4.59 (m, 1H), 3.97 (d, J = 5.1 Hz, 1H), 3.49 (dd, J = 9.8, 4.5 Hz, 1H), 3.30- 3.04 (m, 3H), 3.03-2.80 (m, 1H), 2.75-2.42 (m, 3H), 1.98 (d, J = 6.0 Hz, 3H), 1.46 (d, J = 7.0 Hz, 9H), 1.20-0.94 (m, 1H). LC-MS (ESI) 466.2 [M + 1]. HPLC (220 nm): 98.64%. |
| 2-4 | 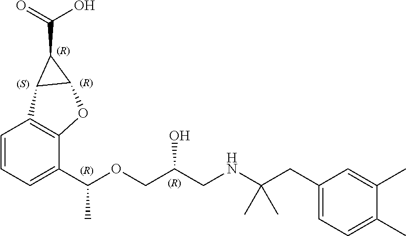 | 381 | ¹H NMR (300 MHz, CD₃OD) δ 1.01-1.02 (m, 1H), 1.28 (s, 6H), 1.40-1.42 (d, J = 6.0 Hz, 3H), 2.24, 2.26 (s, 3H × 2 ), 2.88-2.99 (m, 3H), 3.11-3.12 (m, 1H), 3.22-3.27 (m, 2H), 3.47-3.50 (m, 1H), 3.93-3.95 (m, 1H), 4.61-4.66 (m, 1H), 4.98-5.00 (m, 1H), 6.87-7.12 (m, 5H), 7.31-7.34 (d, 1H); LC-MS: (M + H)⁺ 454; HPLC (220 nm): 98.9%. |
| 2-5 | 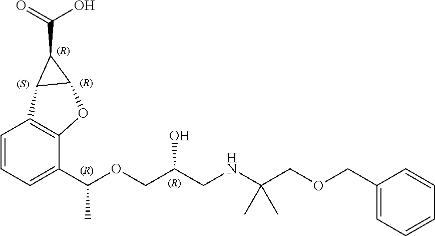 | 4219 | ¹H NMR (300 MHz, CD₃OD) δ 1.10 (m, 1H), 1.34-1.42 (m, 9H), 2.82-2.89 (m, 1H), 3.09- 3.13 (m, 1H), 3.25-3.30 (m, 1H), 3.43-3.49 (m, 3H), 3.95- 3.97 (m, 1H), 4.61-4.66 (m, 3H), 5.11-5.13 (q, J = 6.0 Hz, 1H), 6.94-6.99 (t, 1H), 7.17- 7.19 (m, 1H), 7.31-7.41 (m, 6H); LC-MS: (M + H)⁺ 456; HPLC (220 nm): 99.0%. |
| 2-6 | 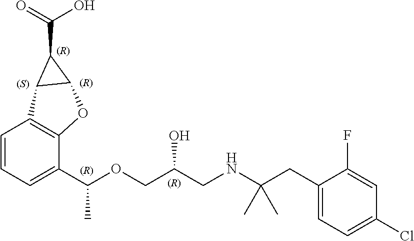 | 730 | ¹H NMR (300 MHz, CD₃OD) δ 1.02 (s, 1H), 1.31 (s, 6H), 1.44-1.46 (d, J = 6.0 Hz, 3H), 2.95-3.14 (m, 4H), 3.25-3.30 (m, 1H), 3.47-3.52 (m, 1H), 3.95-3.97 (m, 1H), 4.61-4.70 (m, 1H), 4.99-5.01 (d, J = 6.0 Hz, 1H), 6.88-6.93 (m, 1H), 7.09-7.36 (m, 5H); LC-MS: (M + H)⁺ 478; HPLC (220 nm): 95.5%. |
| 2-7 | 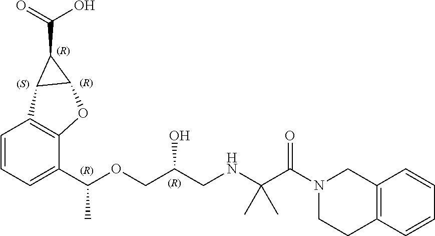 | 10000 | ¹H NMR (300 MHz, CD₃OD) δ 1.11 (m, 1H), 1.42-1.46 (m, 3H), 1.73-1.77 (m, 6H), 1.79- 1.82 (m, 1H), 2.92-3.06 (m, 3H), 3.29-3.30 (m, 1H), 3.47- 3.61 (m, 1H), 3.84-3.92 (m, 2H), 4.04-4.06 (m, 1H), 4.69- 4.71 (m, 1H), 4.77 (s, 2H), 5.11-5.13 (m, 1H,), 6.92-6.99 (m, 1H), 7.20 (s, 5H), 7.30- 7.39 (m, 1H); LC-MS: (M + H)⁺ 495: HPLC (220 nm): 96.7%. |
| 2-8 | 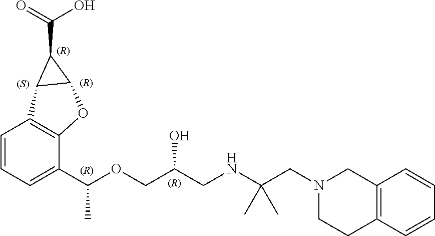 | 7394 | ¹H NMR (300 MHz, CD₃OD) δ 1.12-1.14 (m, 1H), 1.43-1.45 (d, J = 6.0 Hz, 3H), 1.63 (s, 3H), 1.69 (s, 3H), 3.02-3.09 (m, 1H), 3.31-3.32 (m, 4H), 3.45- 3.50 (m, 1H), 3.67 (s, 5H), 4.07-4.09 (m, 1H), 4.57-4.72 (m, 3H), 5.13-5.15 (m, 1H,), 6.95-7.00 (t, 1H), 7.20-7.41 (m, 6H); LC-MS: (M + H)⁺ 481; HPLC (220 nm): 95.2%. |
| 2-9 | 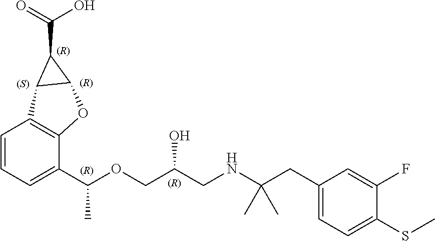 | 830 | ¹HNMR (DMSO, 300 MHz) δ 7.42-7.35 (m, 1H), 7.30-7.25 (m, 1H), 7.20-7.12 (m, 1H), 7.10-7.00 (m, 2H), 7.00-6.88 (m, 1H), 5.25-5.12 (m, 1H), 4.70-4.57 (m, 1H), 3.90 (s, 1H), 3.25-3.18 (m, 2H), 3.10-2.70 (m, 5H), 2.45 (s, 3H), 1.40-1.30 (m, 3H), 1.28- 1.20 (m, 1H), 1.10 (s, 6H). LC-MS (ESI) 490.1 [M + 1]. HPLC (220 nm): 97.86%. |
| 2-10 | 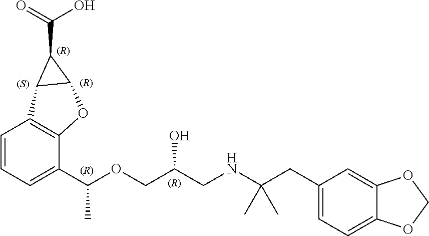 | N/A | ¹H NMR (300 MHz, CD₃OD) δ 7.40-7.35 (m, 1H), 7.20-7.12 (m, 1H), 7.00-6.88 (m, 2H), 6.85-6.60 (m, 2H), 5.82 (s, 2H), 5.20-5.05 (m, 1H), 4.75-4.60 (m, 1H), 4.00-3.60 (m, 1H), 3.58-3.40 (m, 1H), 3.30-3.20 (m, 2H), 3.10-2.85 (m, 4H), 1.46-1.35 (d, 3H), 1.30 (s, 6H), 1.15 (s, 1H); LC-MS: (M + H)⁺ 470; HPLC (220 nm): 95.34%. |
| 2-11 | 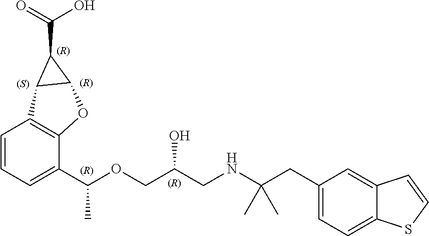 | N/A | ¹H NMR (300 MHz, CD₃OD) δ 7.90-7.85 (m, 1H), 7.75 (s, 1H), 7.65-7.52 (m, 1H), 7.40- 7.35 (m, 2H), 7.30-7.20 (m, 1H), 7.20-7.10 (m, 1H), 7.00-6.90 (m, 1H), 5.18-5.10 (m, 1H), 4.75-4.60 (m, 1H), 4.07-3.88 (m, 1H), 3.60-3.40 (m, 3H), 3.20-2.95 (m, 4H), 1.45-1.25 (m, 9H), 1.18-1.10 (m, 1H); LC-MS: (M + H)⁺ 482; HPLC (220 nm): 97.69%. |

### Example 3

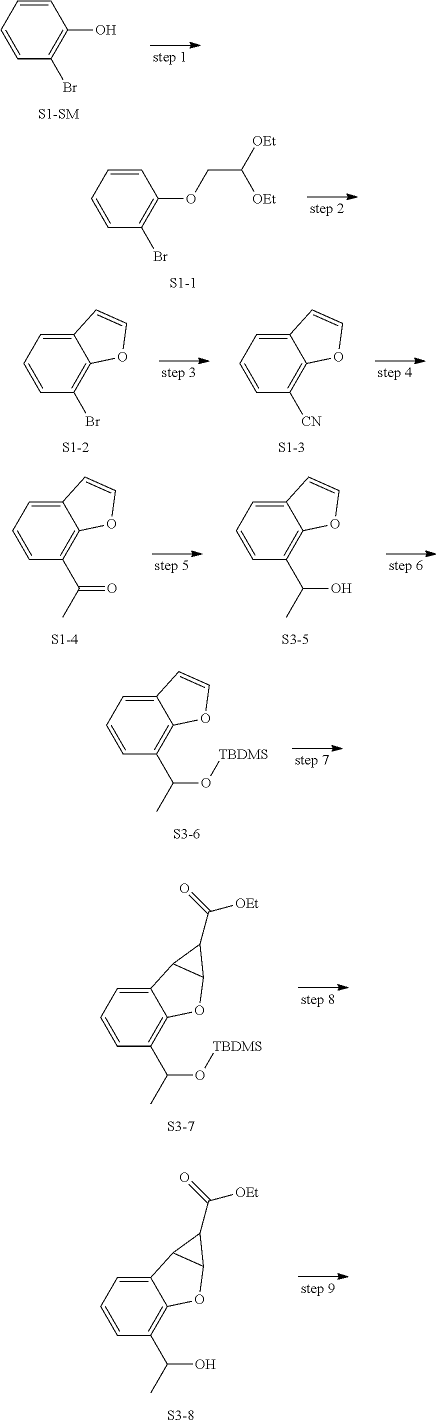

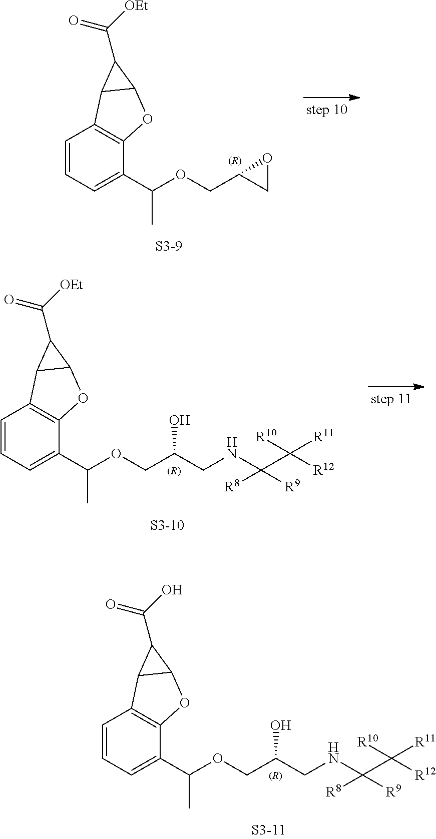

### Experimental Procedure

The experimental procedure in EXAMPLE 3 was the same as that in EXAMPLE 1 except no chiral reagents were used in step 4 and step 7.

The following compounds in Table 3 were made according to EXAMPLE 3.

TABLE 3

| Example | Structure | Analysis Data |
|---|---|---|
| 3-1 | 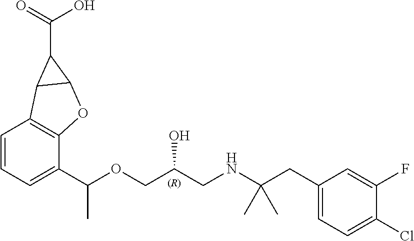 | LC-MS: (M + H)⁺ 478.1 |
| 3-2 | 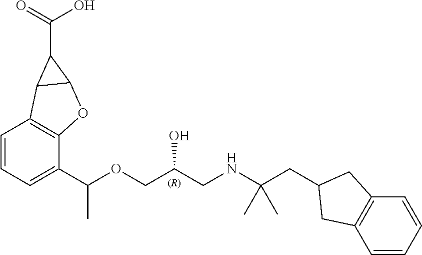 | LC-MS: (M + H)⁺ 466.3 |
| 3-3 | 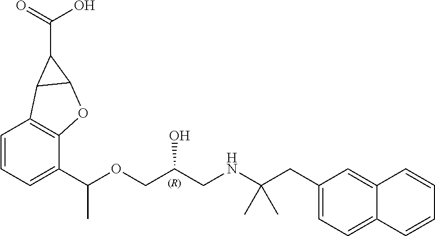 | LC-MS: (M + H)⁺ 476.2 |

### Example 4

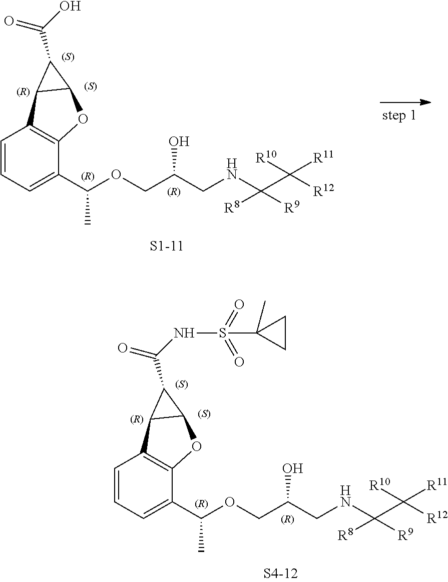

### Experimental Procedure

### Step 1

Oxalyl chloride (0.61 mmol) was added to a solution of S1-11(0.51 mmol) in anhydrous DCM (4 mL) at 0° C. under N₂ atmosphere. After stirring for 30 min, the reaction mixture was evaporated under reduced pressure, and the residue was dissolved in anhydrous DCM (2 mL) and then added dropwise to a solution of 1-methylcyclopropane-1-sulfonamide (0.61 mmol), triethylamine (1.02 mmol), and dimethylaminopyridine (1.02 mmol) in anhydrous DCM (3 mL) at 0° C. The reaction mixture was allowed to warm to room temperature and reacted overnight. The organic solvents were removed in vacuo, and the residue was purified by pre-HPLC to afford S4-12.

The following compound in Table 4 was made according to EXAMPLE 4.

TABLE 4

| | | FLIPR ASSAY | |
|---|---|---|---|
| Example | Structure | IC₅₀ (nM) | Analysis Data |
| 4-1 | 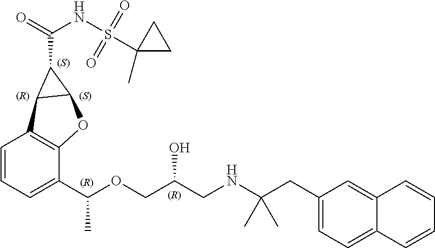 | 60 | ¹H NMR (301 MHz, cd₃od) δ 7.92-7.81 (m, 3H), 7.78 (s, 1H), 7.55-7.44 (m, 2H), 7.44-7.35 (m, 2H), 7.20 (d, J = 6.7 Hz, 1H), 6.99 (t, J = 7.5 Hz, 1H), 5.15 (d, J = 5.4 Hz, 1H), 4.65 (q, J = 6.6 Hz, 1H), 4.01 (s, 1H), 3.49 (dd, J = 9.7, 4.7 Hz, 1H), 3.45-3.34 (m, 2H), 3.21- 3.01 (m, 3H), 1.53 (s, 5H), 1.45-1.23 (m, 9H), 0.95- 0.88 (m, 2H). LC-MS (ESI) 593.2 [M + 1]; HPLC (220 nm): 97.06%. |

### Example 5

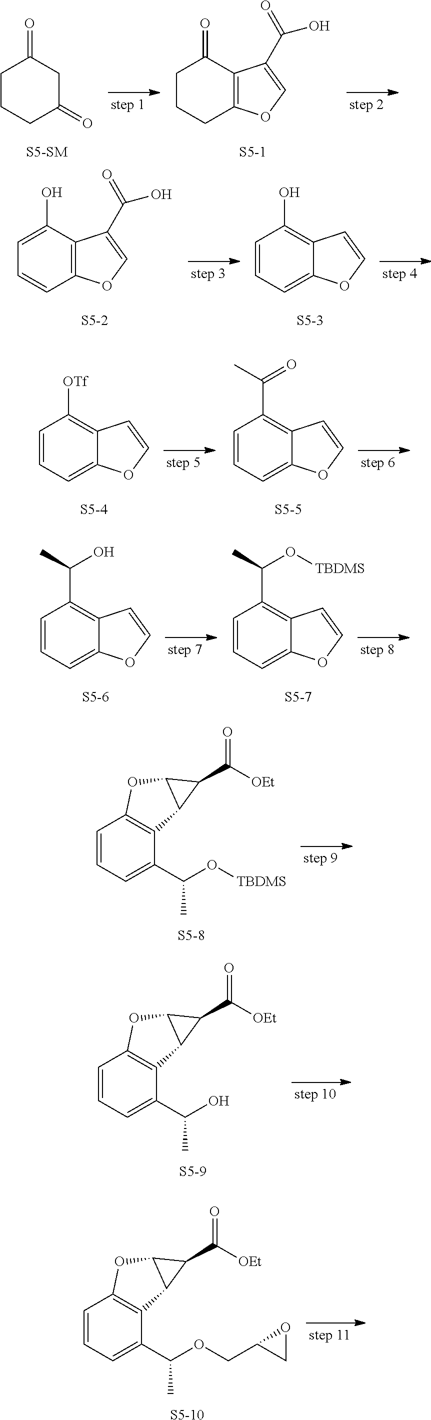

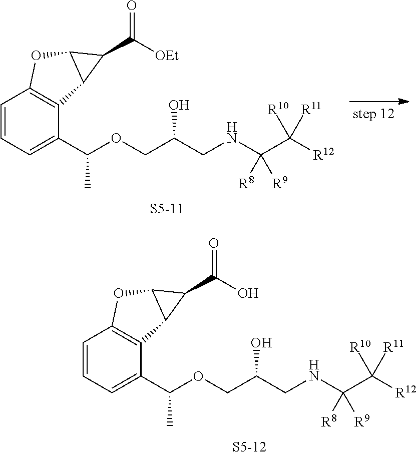

### Experimental Procedure

### Step 1

To a solution of cyclohexane-1,3-dione (S5-SM, 88 g, 786 mmol) in aqueous potassium hydroxide (45 g, 800 mmol) in an ice bath, was added a freshly prepared solution of bromopyruvic acid (131.5 g, 787 mmol) in methanol (400 mL). After the removal of most of the methanol at 30° C. in vacuo, water (800 mL) was added. The pH of the resulting solution was adjusted from ˜2 to 0.2 with concentrated hydrochloric acid and the mixture was heated under reflux at 90-100° C. for 2 hrs. After cooled in an ice bath, the crystalline product was filtered off and dried to give S5-1. ¹H NMR (300 MHz, CDCl₃) δ 13.173 (s, 1H), 8.416 (s, 1H), 2.978-2.936 (m, 2H), 2.613-2.493 (m, 2H), 2.182-2.119 (m, 2H); LC-MS (ESI): 179 [M−H]⁻.

### Step 2

The mixture of S5-1 (20 g, 111 mmol), dodecene (30 mL) and 10% palladium on carbon (10 g, added portionly) were heated in decalin (180 mL) under reflux in a nitrogen atmosphere for 20 hrs. When the mixture had been cooled to ˜80° C., ethanol (300 mL) was added and the cooled mixture was filtered under nitrogen, and washed with ethanol. The filtrate was evaporated and the resulting slurry was cooled to 5° C. Filtration was followed by washing with petroleum ether and air drying gave S5-2. ¹H NMR (300 MHz, CDCl₃) δ 8.602 (s, 1H), 7.280-7.226 (m, 1H), 7.133-7.105 (d, J=8.4 Hz, 1H), 6.699-6.673 (d, J=7.8 Hz, 1H); LC-MS (ESI): 177 [MH]⁻.

### Step 3

S5-2 (20 g, 112 mmol) was stirred with copper powder (20 g, 315 mmol) in quinoline (60 mL) at 230° C. for 5 hrs. The mixture was allowed to cool to about 100° C. and was poured onto crushed ice (500 mL). The mixture was extracted with ether. The combined ether extracts were washed with 2N hydrochloric acid and the brine. The organic layer was separated, dried over anhydrous sodium sulfate, filtered, and concentrated. The product was purified by chromatography (200˜300 mesh silica gel, eluted with petroleum ether/ethyl acetate=6:1) to produce S5-3. ¹H NMR (300 MHz, CDCl₃) δ 7.549-7.542 (d, J=2.1 Hz, 1H), 7.149-7.130 (m, 2H), 6.865-6.867 (d, J=2.4 Hz, 1H), 6.665-6.637 (m, 1H).

### Step 4

To a solution of S5-3 (20 g, 149 mmol) and 2,6-lutidine (40 g, 373 mmol) in CH₂Cl₂ (200 mL) in a nitrogen atmosphere was added triflic anhydride (63 g, 223 mmol) drop-wise under −78° C. The reaction was allowed to proceed for 1 hr at 0° C. After diluted with CH₂Cl₂, the reaction mixture was quenched with water. The organic layer was separated, dried over anhydrous Na₂SO₄, and filtered. The solvent was removed in vacuo to give the product. The product was purified by chromatography (200˜300 mesh silica gel, eluted with petroleum ether/ethyl acetate=100:1) to produce S5-4. ¹H NMR (300 MHz, CDCl₃) δ 7.720-7.691 (d, J=8.7 Hz, 1H)), 7.565-7.537 (d, J=8.4 Hz, 1H), 7.369-7.259 (m, 1H), 7.212-7.185 (d, J=8.1 Hz, 1H), 6.900-6.893 (d, J=2.1 Hz, 1H).

### Step 5

To a mixture of S5-4 (66 g, 248 mmol), 1,3-bis(diphenylphosphino)propane (10.6 g, 25.7 mmol), Pd(OAc)₂ (5.77 g, 25.7 mmol) in ethylene glycol (660 mL) in a nitrogen atmosphere were added 1-(vinyloxy)butane (92.4 g, 923 mmol) and Et₃N (92.4 mL, 640 mmol). The reaction mixture was stirred at 60° C. for 5 hrs. Then HCl (5% aq.) was added, and the mixture was stirred for 30 mins at room temperature. The mixture was diluted with ethyl acetate and washed with brine. The organic layer was separated, filtered and dried over anhydrous Na₂SO₄. The solvent was removed in vacuo to give the product, which was purified by chromatography (200˜300 mesh silica gel, eluted with petroleum ether/ethyl acetate=10:1) to give S5-5. ¹H NMR (300 MHz, CDCl₃) δ 7.815-7.790 (m, 1H), 7.753-7.722 (m, 1H), 7.602-7.575 (m, 1H), 7.398-7.258 (m, 1H), 7.011-7.000 (m, 1H).

### Step 6

(S)-diphenyl prolinol (500 mg) were added to anhydrous tetrahydrofuran, then B(OCH₃)₃ (0.43 mL) were added to the solution at 0° C. ˜−10° C. under N₂. The mixture was stirred overnight. A solution of BH₃.S(CH₃)₂ in THF was added to it. Then a solution of S5-5 (5.0 g, 0.02 mol) in THF was added by syringe pump at 0° C. ˜−10° C. over 5 hrs. TLC indicated the completion of the reaction. The reaction was quenched with HCl (2N aq.), and the mixture was diluted with ethyl acetate. It was washed with aq. NaHCO₃ and brine. The organic layer was separated, dried over anhydrous sodium sulfate, filtered and concentrated to provide a residue, which was purified by column chromatography to afford S5-6. ¹H NMR (300 MHz, CDCl₃) δ 7.82 (d, J=7.5 Hz, 1H), 7.75 (d, J=2.1 Hz, 1H), 7.70 (d, J=8.2 Hz, 1H), 7.55 (dd, J=2.1, 0.8 Hz, 1H), 7.36 (t, J=7.9 Hz, 1H), 2.70 (s, 3H).

### Step 7

To a solution of S5-6 (5.0 g, 0.03 mol) in DMF (50 mL) was added imidazole (5.2 g, 0.075 mol), followed by tert-butyldimethylchlorosilane (TBDMSCl, 5.4 g, 0.036 mol) at 0° C. The mixture was stirred at room temperature for 2 hrs. Then the reaction solution was diluted with ethyl acetate and washed with brine. The organic layer was separated, dried over anhydrous sodium sulfate, filtered and concentrated to provide a residue, which was purified by column chromatography to afford S5-7.

### Step 8

Copper (I) triflate (2:1 complex with toluene, 340 mg, 4%) and (S,S)-(−)-2,2-Isopropylidenebis(4-tert-butyl-2-oxazoline) (0.45 g, 5%, DL Chiral Chemicals) were stirred in dichloromethane (20 mL) at room temperature under N₂ atmosphere overnight. A drop of ethyl diazoethanoate was added to this deep green solution. The color temporarily faded to brown and gas evolving was observed. A solution of S5-7 (8.5 g, 0.031 mol) in dichloromethane (150 mL) was added, followed by a slow addition of a solution of ethyl diazoethanoate (20 mL, 0.13 mol) in DCM (40 mL) during a period of 16 hrs using a syringe pump. The reaction was stirred at room temperature for 2 hrs after the addition. The mixture was concentrated and purified by column chromatography to afford S5-8.

### Step 9

Tetrabutylammonium fluoride (1M in THF, 0.06 mol) was added dropwise to a solution of S5-8 (11.0 g, 0.03 mol) in THF (150 mL) at 0° C. The reaction was stirred at room temperature for 6 hrs. The mixture was filtered and washed with ethyl acetate. The filtrate was washed with brine. The organic layer was separated, dried over anhydrous sodium sulfate, filtered and concentrated to provide a residue, which was purified by column chromatography to afford S5-9. ¹H NMR (300 MHz, CDCl₃) δ 7.19-7.09 (m, 1H), 6.99-6.93 (m, 1H), 6.84-6.76 (m, 1H), 5.16-5.03 (m, 2H), 4.23-4.12 (m, 2H), 3.51-3.44 (m, 1H), 1.55 (dt, J=13.6, 6.8 Hz, 3H), 1.32-1.26 (m, 4H).

### Step 10

To a mixture of S5-9 (4 g, 0.016 mol) and (R)-oxiran-2-ylmethyl 3-nitrobenzenesulfonate (8.35 g, 0.032 mol) in DMF (30 mL) added NaH (1.3 g, 0.032 mol) at 0° C. The reaction was stirred at room temperature for 2 days. The mixture was diluted with ethyl acetate and washed with brine. The organic layer was separated, dried over anhydrous sodium sulfate, filtered and concentrated to provide a residue, which was purified by column chromatography to afford S5-10. ¹H NMR (300 MHz, CDCl₃) δ 7.11 (t, J=7.8 Hz, 1H), 6.82 (dd, J=16.5, 7.8 Hz, 2H), 5.07 (d, J=5.5 Hz, 1H), 4.66 (q, J=6.4 Hz, 1H), 4.24-4.09 (m, 2H), 3.59 (dd, J=11.2, 2.9 Hz, 1H), 3.46 (dd, J=5.4, 3.1 Hz, 1H), 3.24 (dd, J=11.2, 6.3 Hz, 1H), 3.15 (dt, J=6.5, 5.8 Hz, 1H), 2.75 (t, J=4.5 Hz, 1H), 2.50 (dd, J=4.9, 2.6 Hz, 1H), 1.52 (t, J=8.3 Hz, 3H), 1.31-1.22 (m, 4H).

### Step 11

To a solution of S5-10 (100 mg, 0.33 mmol) in DMF (2 mL) was added amine (86 mg, 0.43 mmol). The mixture was stirred at 85° C. overnight. Then the reaction mixture was diluted with ethyl acetate and washed with brine. The organic layer was separated, dried over anhydrous sodium sulfate, filtered and concentrated to provide a residue, which was purified by prep-TLC to afford S5-11.

### Step 12

2N aq. NaOH (0.5 mL) was added to S5-11 (0.207 mmol) in MeOH (1.0 mL) and THF (1.0 mL). The mixture was stirred at 60° C. for 0.5 hrs. Then MeOH and THF were removed under reduced pressure. 1N aq. HCl was added to the reaction in water (10 mL) to make PH=7. The precipitate was collected and purified by prep-HPLC to afford the product S5-12.

The following compounds in Table 5 were made according to EXAMPLE 5.

TABLE 5

| | | FLIPR | |
|---|---|---|---|
| Ex- | | ASSAY | |
| am- | | IC₅₀ | |
| ple | Structure | (nM) | Analysis Data |
| 5-1 | 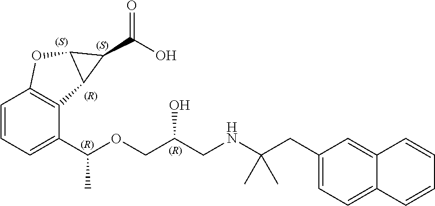 | 9 | ¹H NMR (300 MHz, CD₃OD) δ 7.86 (d, J = 8.3 Hz, 3H), 7.77 (s, 1H), 7.52-7.47 (m, 2H), 7.39 (d, J = 6.7 Hz, 1H), 7.14 (t, J = 7.9 Hz, 1H), 6.84 (dd, J = 14.3, 7.8 Hz, 2H), 5.07 (d, J = 5.5 Hz, 1H), 4.64 (d, J = 6.5 Hz, 1H), 4.00 (s, 1H), 3.46 (m, 5H), 3.14 (s, 2H), 1.48 (d, J = 6.5 Hz, 3H), 1.37 (s, 6H), 1.09 (d, J = 3.1 Hz, 1H). LC-MS (ESI) 476.2 [M + 1]. HPLC (220 nm): 100.00%. |
| 5-2 | 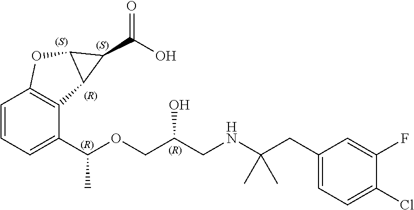 | 32 | ¹H NMR (300 MHz, CD₃OD) δ 7.46 (t, J = 8.1 Hz, 1H), 7.27-7.04 (m, 3H), 6.87 (dd, J = 17.0, 7.9 Hz, 2H), 5.11 (d, J = 5.5 Hz, 1H), 4.66 (t, J = 6.5 Hz, 1H), 3.99 (s, 1H), 3.62-3.44 (m, 2H), 3.42-3.34 (m, 1H), 3.24 (d, J = 3.0 Hz, 1H), 3.14-2.84 (m, 3H), 1.59 (dd, J = 32.0, 6.7 Hz, 3H), 1.31 (s, 6H), 1.13 (d, J = 3.1 Hz, 1H). LC-MS (ESI) 478.1 [M + 1]. HPLC (220 nm): 96.71%. |
| 5-3 | 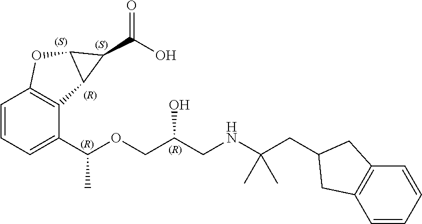 | 35.5 | ¹H NMR (300 MHz, CD₃OD) δ 7.20-7.13 (m, 3H), 7.12-7.07 (m, 2H), 6.90 (d, J = 7.5 Hz, 1H), 6.83 (d, J = 8.1 Hz, 1H), 5.10 (d, J = 5.5 Hz, 1H), 4.67 (q, J = 6.4 Hz, 1H), 3.98 (dd, J = 9.2, 3.1 Hz, 1H), 3.56- 3.46 (m, 2H), 3.35 (d, J = 5.6 Hz, 1H), 3.13 (dd, J = 14.7, 7.2 Hz, 3H), 2.95 (dd, J = 12.4, 9.6 Hz, 1H), 2.61 (ddd, J = 22.4, 15.5, 7.8 Hz, 3H), 1.97 (d, J = 6.1 Hz, 2H), 1.55 (d, J = 6.5 Hz, 3H), 1.43 (s, 6H), 1.12 (d, J = 2.1 Hz, 1H). LC-MS (ESI) 466.2 [M + 1]. HPLC (220 nm): 93.86%. |
| 5-4 | 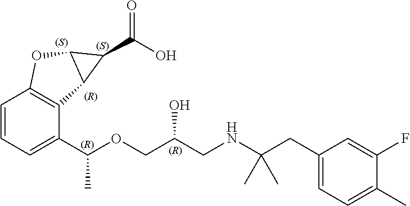 | 36 | ¹H NMR (300 MHz, CD₃OD) δ 7.13 (t, J = 9.0 Hz, 1H), 7.06 (t, J = 9.0 Hz, 1H), 6.92-6.84 (m, 2H), 6.79 (d, J = 9.0 Hz, 1H), 6.72 (d, J = 9.0 Hz, 1H), 4.98 (d, J = 6.0 Hz, 1H), 4.59 (q, J = 6.0 Hz, 1H), 3.94-3.87 (m, 1H), 3.43-3.38 (m, 1H), 3.28-3.25 (m, 1H), 3.19- 3.14 (m, 1H), 3.00-2.90 (m, 2H), 2.86 (s, 2H), 2.17 (s, 3H), 1.45 (d, J = 6.0 Hz, 3H), 1.22 (s, 6H), 1.00 (m, 1H). LC-MS (ESI) 476.2 [M + 1]. HPLC (220 nm): 95.32%. |
| 5-5 | 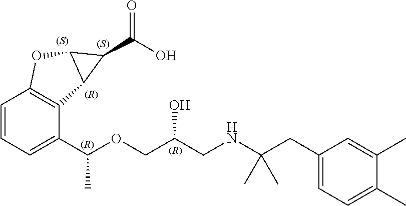 | 26 | ¹H NMR (300 MHz, CD₃OD) δ 7.09-7.04 (m, 1H), 7.04-7.00 (m, 1H), 6.93 (s, 1H), 6.89-6.84 (m, 2H), 6.80-6.77 (m, 1H), 6.75-6.72 (m, 1H), 5.00-4.98 (dd, 1H), 4.57 (q, J = 6.0 Hz, 1H), 3.92-3.88 (m, 1H), 3.43-3.40 (m, 1H), 3.30-3.26 (m, 1H), 3.18-3.13 (m, 1H), 2.97-2.90 (m, 2H), 2.80 (s, 2H), 2.17 (d, J = 6.0 Hz, 6H), 1.42 (d, J = 6.0 Hz, 3H), 1.21 (s, 6H), 1.01 (m, 1H). LC-MS (ESI) 454.2 [M + 1]. HPLC (220 nm): 96.58%. |
| 5-6 | 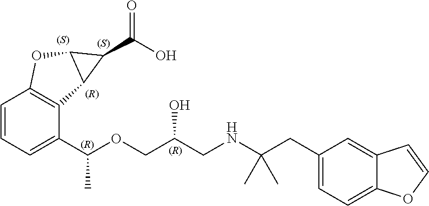 | 137 | ¹H NMR (300 MHz, CD₃OD) δ 7.65 (d, J = 2.1 Hz, 1H), 7.42-7.36 (m, 2H), 7.10-7.03 (m, 2H), 6.78-7.72 (m, 3H), 5.00 (dd, J = 5.4 Hz, 1H), 4.53 (q, J = 6.0 Hz, 1H), 3.94-3.87 (m, 1H), 3.45-3.36 (m, 2H), 3.27- 3.23 (m, 1H), 3.15-3.14 (m, 1H), 2.99-2.92 (m, 3H), 1.38 (d, J = 6.0 Hz, 3H), 1.23 (s, 6H), 1.01 (dd, J = 3.0 Hz, 1H). LC-MS (ESI) 466.1 [M + 1]. HPLC (220 nm): 95.09%. |
| 5-7 | 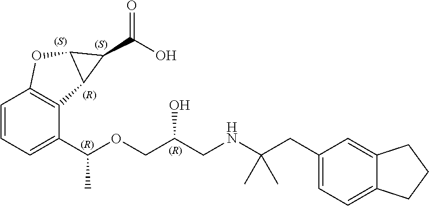 | 25 | ¹H NMR (300 MHz, CD₃OD) δ 7.11-7.01 (m, 3H), 6.92-6.89 (m, 1H), 6.72-6.84 (m, 2H), 4.99 (d, J = 6.0 Hz, 1H), 4.57 (q, J = 6.0 Hz, 1H), 3.94-3.86 (m, 1H), 3.43-3.38 (m, 2H), 3.28-3.25 (m, 1H), 3.19-3.14 (m, 2H), 2.98-2.90 (m, 1H), 2.84-2.78 (m, 5H), 2.04-1.94 (m, 2H), 1.42 (d, J = 6.0 Hz, 3H), 1.22 (s, 6H), 1.01 (m, 1H). LC-MS (ESI) 466 [M + 1]. HPLC (220 nm): 96.03%. |
| 5-8 | 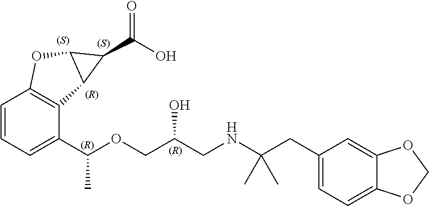 | 160 | ¹H NMR (300 MHz, CD₃OD) δ 6.98 (t, J = 7.8 Hz, 1H), 6.75-6.58 (m, 5H), 5.82 (s, 2H), 4.86 (dd, J = 5.4 Hz, 1H), 4.57 (q, J = 6.6 Hz, 1H), 3.89-3.80 (m, 1H), 3.38-3.33 (m, 1H), 3.24-3.16 (m, 2H), 3.08-3.03 (m, 1H), 2.85-2.81 (m, 1H), 2.74 (s, 2H), 1.42 (d, J = 6.6 Hz, 3H), 1.15 (s, 6H), 0.89 (m, 1H). LC-MS (ESI) 470.1 [M + 1]. HPLC (220 nm): 97.81%. |
| 5-9 | 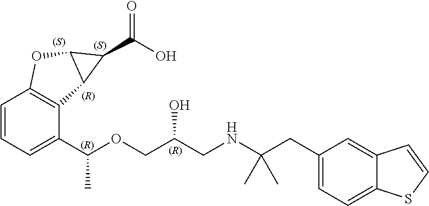 | 23 | ¹H NMR (300 MHz, CD₃OD) δ 7.84 (d, J = 6.0 Hz, 1H), 7.72 (s, 1H), 7.55 (d, J = 6.0 Hz, 1H), 7.34 (d, J = 6.0 Hz, 1H), 7.22 (dd, 1H), 7.07 (t, J = 6.0 Hz, 1H), 6.81 (d, J = 6.0 Hz, 1H), 6.73 (d, J = 6.0 Hz, 1H), 4.96 (dd, 1H), 4.62 (q, J = 6.0 Hz, 1H), 3.96-3.89 (m, 1H), 3.46-3.41 (m, 1H), 3.34-3.32 (m, 1H), 3.29-3.27 (m, 1H), 3.09-3.04 (m, 1H), 2.99 (s, 2H), 2.90-2.84 (m, 1H), 1.47 (d, J = 9.0 Hz, 3H), 1.23 (s, 6H), 1.01 (m, 1H). LC-MS (ESI) 482.2 [M + 1]. HPLC (220 nm): 95.10%. |
| 5-10 | 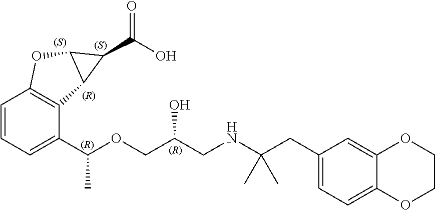 | 153 | ¹H NMR (300 MHz, CD₃OD) δ 7.09 (t, J = 6.0 Hz, 1H), 6.85-6.67 (m, 5H), 4.98-4.96 (m, 1H), 4.68 (q, J = 6.0 Hz, 1H), 4.22 (s, 4H), 4.00-3.93 (m, 1H), 3.49- 3.44 (m, 1H), 3.35-3.32 (m, 1H), 3.28-3.26 (m, 1H), 3.22-3.17 (m, 1H), 2.98-2.91 (m, 1H), 2.83 (s, 2H), 1.52 (d, J = 6.0 Hz, 3H), 1.27 (s, 6H), 1.00 (m, 1H). LC-MS (ESI) 484.2 [M + 1]. HPLC (220 nm): 97.30%. |
| 5-11 | 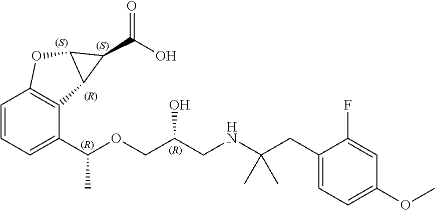 | 108 | ¹H NMR (300 MHz, CD₃OD) δ 7.07 (t, J = 8.4 Hz, 1H), 6.98 (t, J = 7.8 Hz, 1H), 6.73 (d, J = 7.5 Hz, 1H), 6.67-6.58 (m, 3H), 4.86 (d, J = 5.4 Hz, 1H), 4.57 (q, J = 6.3 Hz, 1H), 3.89-3.76 (m, 1H), 3..69 (s, 3H), 3.37-3.32 (m, 1H), 3.24-3.16 (m, 2H), 3.04-2.99 (m, 1H), 2.82-2.71 (m, 3H), 1.43 (d, J = 6.6 Hz, 3H), 1.12 (s, 6H), 0.89 (m, 1H). LC-MS (ESI) 474.1 [M + 1]. HPLC (220 nm): 95.32%. |
| 5-12 | 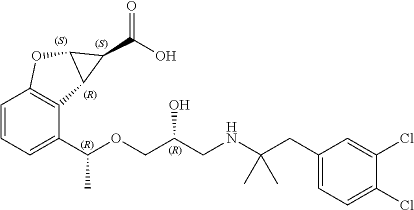 | 39 | ¹H NMR (300 MHz, CD₃OD) δ 7.39-7.34 (m, 2H), 7.08 (dd, J = 8.4 Hz, 1H), 6.98 (t, J = 7.8 Hz, 1H), 6.74 (d, J = 7.5 Hz, 1H), 6.63 (d, J = 7.8 Hz, 1H), 4.86 (dd, J = 5.4 Hz, 1H), 4.57 (q, J = 6.6 Hz, 1H), 3.89-3.81 (m, 1H), 3.38-3.33 (m, 1H), 3.25-3.17 (m, 2H), 3.07-3.02 (m, 1H), 2.89-2.77 (m, 3H), 1.43 (d, J = 6.6 Hz, 3H), 1.14 (s, 6H), 0.90 (dd, J = 3.0 Hz, 1H). LC-MS (ESI) 494.0 [M + 1]. HPLC (220 nm): 98.23%. |
| 5-13 | 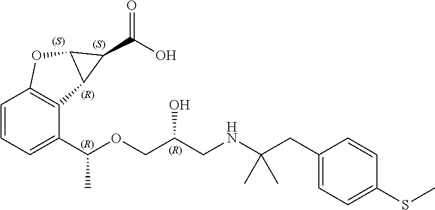 | 72 | ¹H NMR (300 MHz, CD₃OD) δ 7.14-7.05 (m, 4H), 6.98 (t, J = 7.8 Hz, 1H), 6.73 (d, J = 7.5 Hz, 1H), 6.64 (d, J = 8.1 Hz, 1H), 4.86 (dd, J = 5.4 Hz, 1H), 4.56 (q, J = 6.6 Hz, 1H), 3.87-3.80 (m, 1H), 3.37-3.32 (m, 1H), 3.24-3.17 (m, 2H), 3.05-3.00 (m, 1H), 2.83-2.76 (m, 3H), 2.35 (s, 3H), 1.41 (d, J = 6.6 Hz, 3H), 1.13 (s, 6H), 0.90 (d, J = 2.4 Hz, 1H). LC-MS (ESI) 472.1 [M + 1]. HPLC (220 nm): 95.18%. |
| 5-14 | 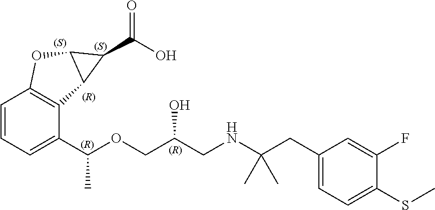 | 27 | ¹H NMR (300 MHz, CD₃OD) δ 7.21 (t, J = 6.0 Hz, 1H), 7.07 (t, J = 6.0 Hz, 1H), 6.98-6.89 (m, 2H), 6.80- 6.72 (m, 2H), 5.01 (m, 1H), 4.56 (q, J = 6.0 Hz, 1H), 3.93-3.85 (m, 1H), 3.46-3.37 (m, 1H), 3.28-3.24 (m, 1H), 3.19-3.14 (m, 1H), 2.99-2.91 (m, 1H), 2.86 (s, 2H), 2.37 (s, 3H), 1.43 (d, J = 6.0 Hz, 3H), 1.21 (s. 6H), 1.03 (m, 1H). LC-MS (ESI) 489.9 [M + 1]. HPLC (220 nm): 99.34%. |
| 5-15 | 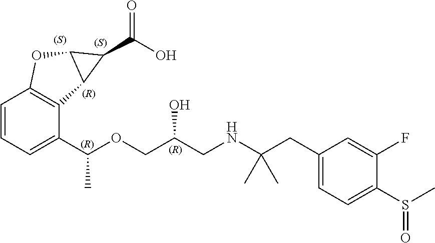 | >10000 | ¹H NMR (300 MHz, CD₃OD) δ 7.78 (t, J = 6.0 Hz, 1H), 7.39 (d, J = 9.0 Hz, 1H), 7.26-7.14 (m, 2H), 6.91-6.83 (m, 2H), 5.11 (d, J = 3.0 Hz, 1H), 4.69 (q, J = 6.0 Hz, 1H), 4.05-3.98 (m, 1H), 3.56-3.48 (m, 1H), 3.39-3.35 (m, 1H), 3.29-3.26 (m, 1H), 3.16-2.97 (m, 3H), 2.89 (s, 3H), 1.55 (d, J = 6.0 Hz, 3H), 1.33 (s, 6H), 1.13 (m, 1H). LC-MS (ESI) 506.1 [M + 1]. HPLC (220 nm): 99.07%. |
| 5-16 | 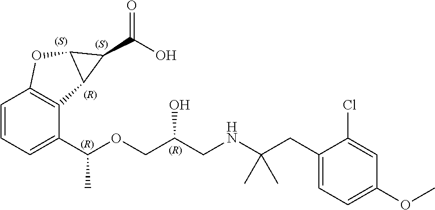 | 38 | ¹H NMR (300 MHz, CD₃OD) 7.27 (d, J = 9.0 Hz, 1H), 7.09 (t, J = 6.0 Hz, 1H), 6.90-6.84 (m, 2H), 6.75 (d, J = 9.0 Hz, 2H), 4.97 (d, J = 6.0 Hz, 1H), 4.70 (q, J = 6.0 Hz, 1H), 4.05-3.95 (m, 1H), 3.79 (s, 3H), 3.52- 3.46 (m, 1H), 3.37-3.33 (m, 1H), 3.29-3.22 (m, 1H), 3.10 (d, J = 3.0 Hz, 1H), 3.05-2.95 (m, 2H), 1.54 (d, J = 9.0 Hz, 3H), 1.31 (s, 6H), 1.01 (m, 1H). LC-MS (ESI) 490.1 [M + 1]. HPLC (220 nm): 96.74% |
| 5-17 | 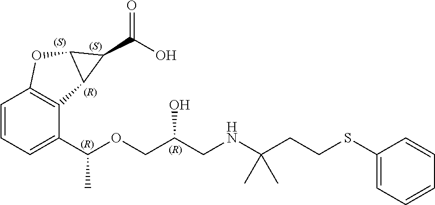 | 150 | ¹H NMR (300 MHz, CD₃OD) δ 7.40-7.37 (m, 2H), 7.29 (t, J = 7.5 Hz, 2H), 7.22-7.17 (m, 1H), 7.08 (t, J = 7.8 Hz, 1H), 6.83 (d, J = 7.5 Hz, 1H), 6.75 (d, J = 7.8 Hz, 1H), 4.96 (d, J = 8.4 Hz, 1H), 4.67 (q, J = 6.3 Hz, 1H), 3.95-3.87 (m, 1H), 3.44-3.39 (m, 1H), 3.28-3.24 (m, 2H), 3.06-2.95 (m, 3H), 2.80-2.73 (m, 1H), 1.93 (t, J = 8.7 Hz, 2H), 1.53 (d, J = 6.3 Hz, 3H), 1.33 (s, 6H), 0.99 (d, J = 2.4 Hz, 1H). LC-MS (ESI) 472.2 [M + 1]. HPTC (220 nm): 96.69%. |
| 5-18 | 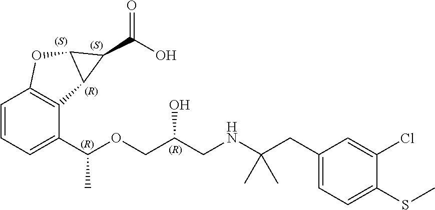 | 53 | ¹H NMR (300 MHz, CD₃OD) δ 7.30-7.17 (m, 4H), 6.90-6.82 (m, 2H), 5.11 (d, J = 5.1 Hz, 1H), 4.65 (q, J = 6.6 Hz, 1H), 4.03-3.96 (m, 1H), 3.56-3.47 (m, 2H), 3.38-3.23 (m, 2H), 3.08-3.94 (m, 3H), 2.48 (s, 3H), 1.52 (d, J = 6.3 Hz, 3H), 1.31 (s, 6H), 1.13 (d, J = 2.4 Hz, 1H). LC-MS (ESI) 506.2 [M + 1]. HPLC (220 nm): 95.20%. |
| 5-19 | 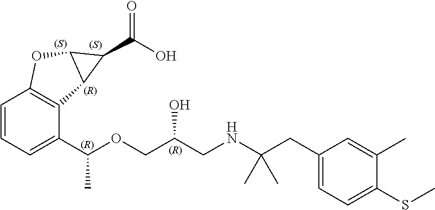 | 97 | ¹H NMR (300 MHz, CD₃OD) δ 7.17 (t, J = 7.8 Hz, 2H), 7.10-7.05 (m, 2H), 6.90-6.83 (m, 2H), 5.11 (d, J = 5.4 Hz, 1H), 4.64 (q, J = 7.2 Hz, 1H), 4.02-3.95 (m, 1H), 3.55-3.47 (m, 2H), 3.37-3.22 (m, 1H), 3.08- 3.00 (m, 2H), 2.90 (s, 2H), 2.45 (s, 3H), 2.29 (s, 3H), 1.51 (d, J = 6.6 Hz, 3H), 1.30 (s, 6H), 1.12 (m, 1H). LC-MS (ESI) 486.1 [M + 1]. HPLC (220 nm): 97.46%. |
| 5-20 | 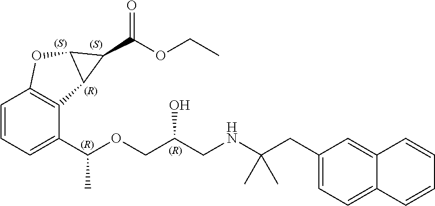 | 130 | ¹H NMR (300 MHz, CDCl₃) δ 7.88-7.70 (m, 4H), 7.52-7.44 (m, 2H), 7.36 (d, J = 8.4 Hz, 1H), 7.06 (t, J = 7.8 Hz, 1H), 6.78 (dd, J = 7.7, 3.6 Hz, 2H), 5.05 (d, J = 5.6 Hz, 1H), 4.58 (q, J = 6.4 Hz, 1H), 4.48 (s, 1H), 4.14 (m, 1H), 3.52-3.47 (m, 1H), 3.40-3.37 (m, 2H), 3.32 (s, 2H), 3.10-3.00 (m, 1H), 1.43 (t, J = 8.3 Hz, 8H), 1.30-1.20 (m, 5H). LCMS (ESI): 504.3 [M + 1]. HPLC (220 nm): 98.46%. |

### Example 6

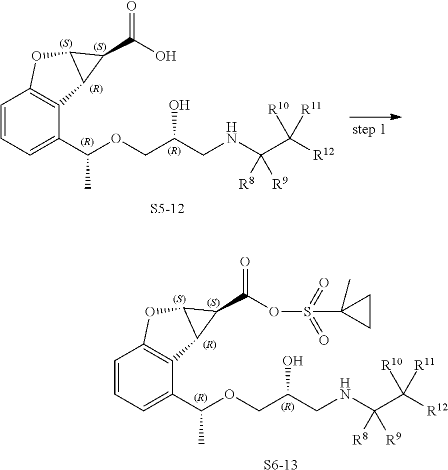

### Experimental Procedure

The experimental procedure in EXAMPLE 6 employed the same methods as that in EXAMPLE 4.

The following compound in Table 6 was made according to EXAMPLE 6.

TABLE 6

| | | FLIPR | |
|---|---|---|---|
| Exam- | | ASSAY | |
| ple | Structure | IC₅₀ (nM) | Analysis Data |
| 6-1 | 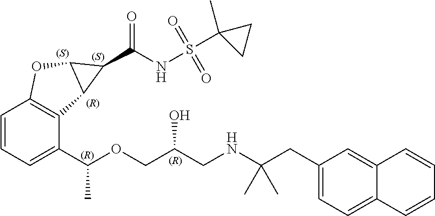 | 30 | ¹H NMR (CDCl₃, 300 MHz) δ 7.87-7.84 (m, 3H), 7.77 (s, 1H), 7.50-7.47 (m, 2H), 7.41-7.38 (m, 1H), 7.20-7.15 (m, 1H), 6.89-6.84 (m, 2H), 5.14 (d, J = 5.5 Hz, 1H), 4.62 (q, J = 6.4 Hz, 1H), 4.04 (m, 1H), 3.65 (dd, J = 5.4, 3.0 Hz, 1H), 3.51 (dd, J = 9.7, 5.0 Hz, 1H), 3.35-3.31 (m, 2H), 3.18-3.01 (m, 3H), 1.58- 1.52 (m, 4H), 1.49 (d, J = 6.4 Hz, 3H), 1.38 (s, 6H), 1.34-1.29 (m, 2H), 0.91 (t, J = 6.4 Hz, 2H): LC-MS (ESI) 593.3 [M + 1]; HPLC (220 nm): 97.3%. |

### Example 7

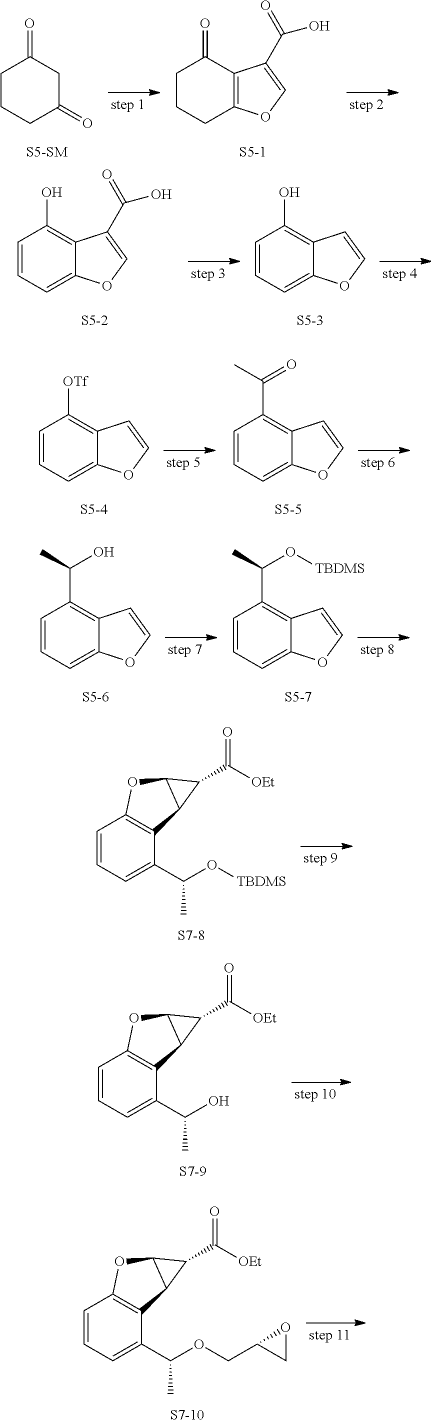

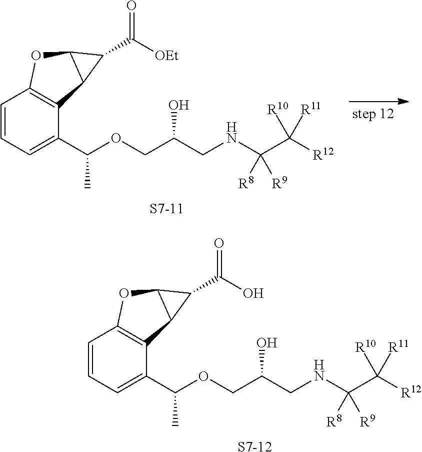

### Experimental Procedure

The experimental procedure in EXAMPLE 7 was the same as that in EXAMPLE 5 except (R,R)-(+)-2,2-Isopropylidenebis(4-tert-butyl-2-oxazoline) was used in step 8.

The following compound in Table 7 was made according to EXAMPLE 7.

TABLE 7

| | | FLIPR | |
|---|---|---|---|
| Ex- | | ASSAY | |
| am- | | IC₅₀ | |
| ple | Structure | (nM) | Analysis Data |
| 7-1 | 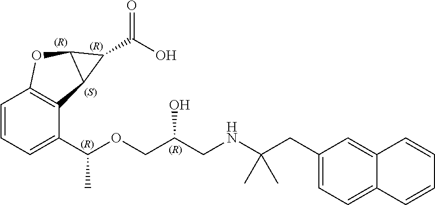 | 80 | ¹H NMR (300 MHz, CD₃OD) δ 7.85 (dd, J = 8.8, 2.8 Hz, 3H), 7.77 (s, 1H), 7.54-7.44 (m, 2H), 7.39 (dd, J = 8.5, 1.8 Hz, 1H), 7.17( t, J = 7.9 Hz, 1H), 6.89 (d, J = 7.5 Hz, 1H), 6.81 (d, J = 8.0 Hz, 1H), 5.13 (dd, J = 5.4, 1.1 Hz, 1H), 4.70 (q, J = 6.5 Hz, 1H), 4.04 (dd, J = 9.0, 3.1 Hz, 1H), 3.48-3.40 (m, 3H), 3.21-3.03 (m, 3H), 1.42 (s, 3H), 1.37 (s, 6H), 1.11 (dd, J = 3.1, 1.1 Hz, 1H). LC-MS (ESI) 476.2 [M + 1]. HPLC (220 nm): 96.26%. |

### Example 8

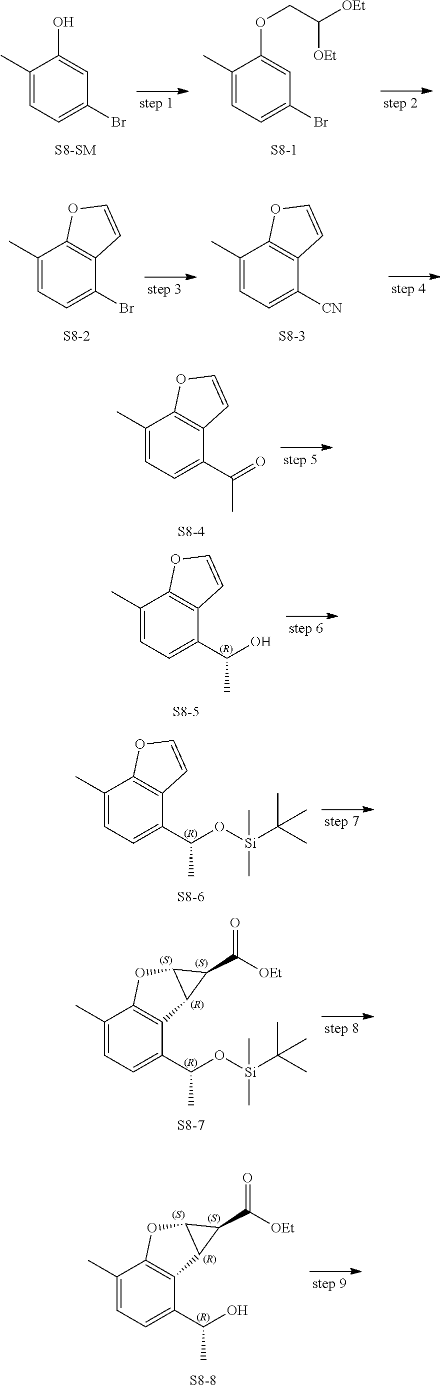

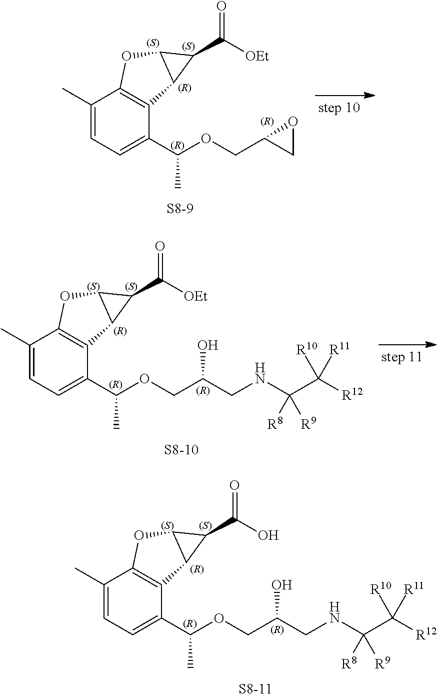

### Experimental Procedure

### Step 1

The suspension of S8-SM (23 g, 124 mmol) and K₂CO₃ (34.3 g, 248 mmol) in DMF was stirred at 100° C. for 1 hr, then 2-bromo-1,1-diethoxyethane (21 mL, 155 mmol) was added dropwise. The reaction mixture was stirred at 100° C. overnight. The reaction mixture was cooled to room temperature and filtered. The filtrate was poured into water and the mixture was extracted with ethyl acetate. The combined organic layer was washed with water and brine two times respectively, separated, dried over anhydrous sodium sulfate, filtered and concentrated. The obtained residue was purified by chromatography and eluted with petroleum ether:ethyl acetate (60:1) to afford S8-1. ¹H NMR (CDCl₃, 300 MHz) δ 6.96-6.95 (m, 2H), 6.92 (s, 1H), 4.83-4.80 (t, J=5.1 Hz, 1H,) 3.96-3.95 (d, J=5.4 Hz, 2H), 3.81-3.57 (m, 4H), 2.15 (s, 3H), 1.26-1.21 (t, J=6.9 Hz, 6H).

### Step 2

The mixture of S8-1 (25.2 g, 87.8 mmol) and polyphosphoric acid (44.5 g, 132 mmol) in chlorobenzene (100 mL) was stirred at 80° C. for 3 hrs, then the reaction mixture was cooled to room temperature. The mixture was concentrated and the black residue was disposed with aq. Na₂CO₃, and extracted with ethyl acetate. The combined organic layer was washed with aq. Na₂CO₃, separated, dried over anhydrous sodium sulfate, filtered and concentrated. The obtained residue was purified by chromatography to afford S8-2. ¹H NMR (CDCl₃, 300 MHz) δ 7.641-7.640 (d, J=1.8 Hz, 1H), 7.28-7.26 (d, J=7.8 Hz, 1H), 6.96-6.94 (d, J=8.1 Hz, 1H), 6.78-6.77 (d, J=1.8 Hz, 1H), 2.47 (s, 3H).

### Step 3

A mixture of S8-2 (16.3 g, 78 mmol), CuCN (21.1 g, 234 mmol) and CuI (29.8 g, 156 mmol) in DMF (140 mL) was stirred at 150° C. for 12 hrs. The reaction mixture was cooled to room temperature and filtered. The filtrate was poured into water and extracted with ethyl acetate. The combined organic layer was washed with water and brine two times respectively, separated, dried over anhydrous sodium sulfate, filtered and concentrated. The obtained residue was purified by chromatography and eluted with petroleum ether:ethyl acetate (60:1) to afford S8-3. ¹H NMR (CDCl₃, 300 MHz) δ 7.76-7.75 (d, J=2.1 Hz, 1H), 7.48-7.45 (d, J=7.8 Hz, 1H), 7.15-7.12 (d, J=7.8 Hz, 1H), 6.96-6.95 (d, J=2.4 Hz, 1H), 2.58 (s, 3H).

### Step 4

To the solution of S8-3 (6 g, 38.2 mmol) in toluene (60 mL) was added dropwise methylmagnesium bromide (38 mL, 3.0 M in ether, 114.6 mmol) at room temperature under N₂. The solution was stirred at 60° C. for 1 hr. NH₄Cl (aq.) was added to the solution and the mixture was acidified with 1N HCl. The resulting mixture was refluxed for 1 hr, and then diluted with ether, washed with saturated aq. NaHCO₃ and brine. The organic layer was separated, dried over anhydrous sodium sulfate, filtered and concentrated to provide a residue, which was purified by column chromatography to afford S8-4. ¹H NMR (CDCl₃, 300 MHz) δ 7.73-7.70 (m, 2H), 7.53 (d, J=2.1 Hz, 1H), 7.13 (d, J=7.5 Hz, 1H), 2.65 (s, 3H), 2.58 (s, 3H).

### Step 5

To a solution of (S)-diphenyl prolinol (0.746 g, 3.9 mmol) in THF (50 mL) was added BH₃.Me₂S (1.56 g, 39 mmol). The mixture was stirred at room temperature for 2 hrs. Then S8-4 (6.8 g, 39 mmol) was added to the solution for another 20 hrs. The reaction was quenched with diluted HCl, and concentrated. The residue was poured into water and extracted with ethyl acetate. The combined organic layer was washed with water and brine two times respectively, separated, dried over anhydrous sodium sulfate, filtered and concentrated. The obtained residue was purified by chromatography and eluted with petroleum ether:ethyl acetate (5:1) to afford product S8-5. ¹H NMR (CDCl₃ 300 MHz) δ 7.62-7.61 (d, J=1.8 Hz, 1H), 7.12-7.03 (m, 2H), 693-6.92-7.12 (d, J=7.8 Hz, 1H), 5.18-5.12 (q, J=6.3 Hz, 1H), 2.51 (s, 3H), 1.58-1.56 (d, J=6.6 Hz, 3H).

### Step 6

To a solution of S8-5 (4.95 g, 28.1 mmol) in DMF (50 mL) was added imidazole (3.8 g, 56.2 mmol), followed by tert-butyldimethylchlorosilane (TBDMSCl, 4.2 g, 28.1 mmol) at 0° C. The mixture was stirred at room temperature for 2 hrs. Then the reaction mixture was diluted with ethyl acetate and washed with brine. The organic layer was separated, dried over anhydrous sodium sulfate, filtered and concentrated to provide a residue, which was purified by column chromatography to afford S8-6. ¹H NMR (CDCl₃, 300 MHz) δ 7.60 (d, J=2.4 Hz, 1H), 7.08-7.00 (m, 2H), 7.00 (d, J=2.1 Hz, 1H), 5.08 (q, J=6.3 Hz, 1H), 2.50 (s, 3H), 1.48 (s, J=6.3 Hz, 3H), 0.926 (s, 9H), 0.057 (s, 3H), −0.093 (s, 3H).

### Step 7

Copper (I) triflate (2:1 complex with toluene, 82 mg, 0.31 mmol) and (S,S)-(−)-2,2-Isopropylidenebis(4-tert-butyl-2-oxazoline) (0.114 g, 0.388 mmol, DL Chiral Chemicals) were stirred in dichloromethane (20 mL) at room temperature under N₂ for 1 hr. A drop of ethyl diazoethanoate was added to this deep green solution. The color temporarily faded to brown and gas evolving was observed. A solution of S8-6 (4.5 g, 0.0155 mol) in dichloromethane (80 mL) was poured into it, followed by a slow addition of a solution of ethyl diazoethanoate (9 mL, 0.067 mol) in DCM (40 mL) during a period of 16 hrs using a syringe pump. The mixture was stirred at room temperature for 2 hrs after the addition. The mixture was concentrated and purified by column chromatography to afford S8-7, which was directly used in the next step.

### Step 8

Tetrabutylammonium fluoride (30.36 mL, 1 M in THF, 0.023 mol) was added dropwise to a solution of S8-7 (5.9 g, 0.016 mol) in THF (60 mL) at 0° C. The reaction was stirred at room temperature overnight, quenched with water and extracted with ethyl acetate. The organic layer was separated, dried over anhydrous sodium sulfate, filtered and concentrated under reduced pressure to provide a residue, which was purified by chromatography and eluted with petroleum ether:ethyl acetate (5:1) to afford S8-8. ¹H NMR (CDCl₃, 300 MHz) δ 6.97-6.82 (m, 2H), 5.10-5.03 (m, 2H), 4.191-4.11 (m, 2H), 3.46-3.35 (m, 1H), 2.18 (s, 3H), 1.88 (s, 1H), 1.53-1.50 (s, 3H), 1.26 (t, J=6.9 Hz, 3H), 1.21 (d, J=3.0 Hz, 1H).

### Step 9

To a mixture of S8-8 (0.7 g, 2.65 mmol) and (R)-oxiran-2-ylmethyl 3-nitrobenzenesulfonate (0.76 g, 2.92 mmol) in DMF (10 mL) was added NaH (0.12 g, 3 mmol) at 0° C. The reaction was stirred at room temperature for 2 days. The mixture was diluted with ethyl acetate and washed with brine. The organic layer was separated, dried over anhydrous sodium sulfate, filtered and concentrated to provide a residue, which was purified by column chromatography to afford S8-9. ¹H NMR (CDCl₃, 300 MHz) δ 6.93-6.74 (m, 2H), 5.05 (d, J=5.4 Hz, 2H), 4.67-4.61 (m, 1H), 4.15 (q, J=6.9 Hz, 2H), 3.63-3.60 (m, 1H), 3.43-3.41 (m, 1H), 3.20-3.10 (m, 2H), 2.74-2.71 (m, 1H), 2.48-2.47 (m, 1H), 2.17 (s, 3H), 1.49-1.46 (m, 3H), 1.27-1.22 (m, 3H), 1.16-1.13 (m, 1H).

### Step 10

To a solution of S8-9 (100 mg, 0.3 mmol) in DMF (2 mL) was added amine (126 mg, 0.629 mmol) at room temperature. The solution was stirred overnight. Then the reaction mixture was added with water and extracted with ethyl acetate. The combined organic layers were washed with brine. The organic layer was separated, dried over anhydrous sodium sulfate, filtered and concentrated to provide a residue, which was purified by prep-TLC to afford S8-10.

### Step 11

2N aq. NaOH (0.5 mL) was added to S8-10 (0.07 mmol) in MeOH (1.0 mL) and THF (1.0 mL). The mixture was stirred at 60° C. for 45 mins. Then MeOH and THF were removed under reduced pressure. 1N aq. HCl was added to the reaction in water (10 mL) to make PH=7. The precipitate was collected and purified by prep-HPLC to afford the product S8-11.

The following compounds in Table 8 were made according to EXAMPLE 8.

TABLE 8

| | | FLIPR | |
|---|---|---|---|
| Ex- | | ASSAY | |
| am- | | IC₅₀ | |
| ple | Structure | (nM) | Analysis Data |
| 8-1 | 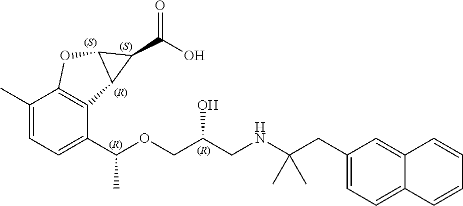 | 14 | ¹H NMR (CD3OD, 300 MHz) δ 7.87-7.84 (m, 3H), 7.76 (s, 1H), 7.50-7.38 (m, 3H), 6.96-6.94 (d, 1H), 6.78-6.75 (d, J = 9.0 Hz, 1H), 5.12-5.10 (d, J = 6.0 Hz, 1H), 4.59-4.57 (m, 1H), 4.00 (m, 1H), 3.50-3.30 (m, 4H), 3.15-3.04 (m, 3H), 2.18 (s, 3H), 1.48-1.45 (d, J = 9.0 Hz, 3H), 1.36 (s, 6H), 1.10-1.09 (m, 1H): LC-MS (ESI): 490.3 [M + 1]; HPLC (220 nm): 96.7%. |
| 8-2 | 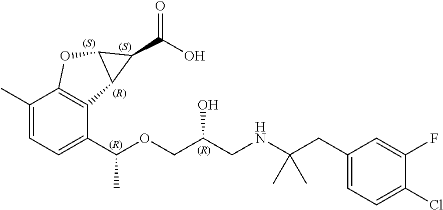 | 58.5 | ¹H NMR (CD3OD, 300 MHz) δ 7.46-7.43 (t, J = 7.8 Hz, 1H), 7.23 (d, 1H), 7.15 (d, 1H), 7.86-7.92 (d, 1H), 6.65-6.63 (d, 1H), 5.03-5.00 (d, 1H), 4.59-4.57 (m, 1H), 4.02 (m, 1H), 3.32-3.18 (m, 3H), 3.00-2.92 (m, 3H), 2.15 (s, 3H), 1.50-1.47 (m, 3H), 1.29 (s, 6H), 0.89-0.88 (m, 1H); LC-MS (ESI): 492.2 [M + 1]; HPLC (220 nm): 96.4%. |
| 8-3 | 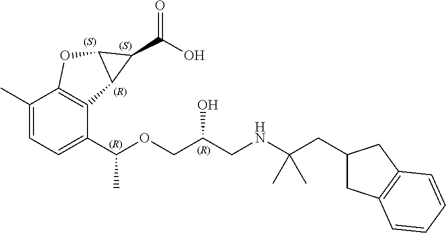 | 20 | ¹H NMR (CD3OD, 300 MHz) δ 7.15-7.05 (m, 4H), 6.90-6.87 (d, J = 9.0 Hz, 1H), 6.75-6.72 (d, J = 9.0 Hz, 1H), 4.97-4.95 (m, 1H), 4.64-4.62 (m, 1H), 3.91- 3.89 (m, 1H), 3.41-3.33 (m, 2H), 3.16-3.02 (m, 3H), 2.89-2.61 (m, 4H), 2.14 (s, 3H), 1.89 (d, 2H), 1.55- 1.52 (d, J = 9.0 Hz, 3H), 1.33 (s, 6H), 1.00-0.98 (m, 1H); LC-MS (ESI): 480.3 [M + 1]; HPLC (220 nm): 95.7%. |
| 8-4 | 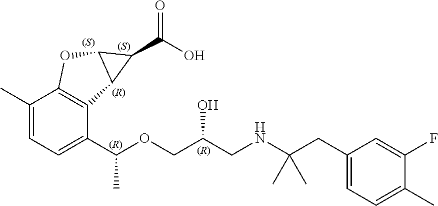 | 21 | ¹H NMR (CD3OD, 300 MHz) δ 7.50-7.48 (d, 2H, J = 6.0 Hz), 6.92-6.88 (m, 3H), 6.74-6.72 (d, 1H, J = 6.0 Hz), 4.99-4.98 (m, 1H), 4.63 4.61 (m, 1H), 3.87 (m,1H), 3.35 (m, 1H), 3.21 (m, 1H), 2.76-2.75 (m, 5H), 2.23 (s, 3H), 2.15 (s, 3H), 1.52-1.50 (d, 3H, J = 6.0 Hz), 1.14 (s, 6H), 0.98-0.96 (m, 1H): LC-MS (ESI): 472.2 [M + 1]; HPLC (220 nm): 95.8%. |
| 8-5 | 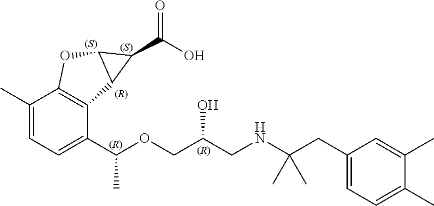 | 18 | ¹H NMR (CD3OD, 300 MHz) δ 7.09 (d, J = 7.6 Hz. 1H), 7.01 (s, 1H), 6.98-6.96 (m, 2H), 6.80-6.76 (m, 1H), 5.10 (dd, J = 5.5, 1.0 Hz, 1H), 4.60 (q, J = 6.4 Hz, 1H), 4.07-3.95 (m, 1H), 3.56-3.38 (m, 2H), 3.40- 3.34 (m 1H), 3.28-3.19 (m, 1H), 3.07-2.83 (m, 3H), 2.25 (d, J = 5.7 Hz, 6H), 2.18 (s, 3H), 1.49 (d, J = 6.4 Hz, 2H), 1.47-1.44 (m, 1H), 1.30 (d, J = 4.6 Hz, 6H), 1.13-1.05 (m, 1H); LC-MS (ESI): 468.2 [M + 1]; HPLC (220 nm): 94.8%. |
| 8-6 | 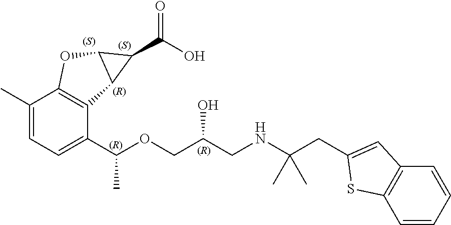 | 96 | ¹H NMR (CD3OD), 300 MHz) δ 7.82-7.73 (m, 1H), 7.32 (m, 3H), 7.20 (s, 1H), 6.92-6.75 (d, J = 6.9 Hz, 1H), 6.75-6.52 (d, J = 6.9 Hz, 1H), 5.36 (t, 1H), 4.87 (m, 1H), 4.58 (m, 1H), 3.82 (s, 1H), 67 (s, 1H), 3.34-3.33 (m, 1H), 3.13 (m, 5H), 3.13 (m, 5H), 2.94 (s ,1H), 2.77-2.68 (m, 1H), 2.19 (s, 3H), 2.04 (m, 3H), 1.47 (d, J = 9.0 Hz, 3H), 1.32 (s, 6H), 1.13-1.10 (m, 1H); LC-MS (ESI): 496.2 [M + 1]; HPLC (220 nm): 95.3%. |
| 8-7 | 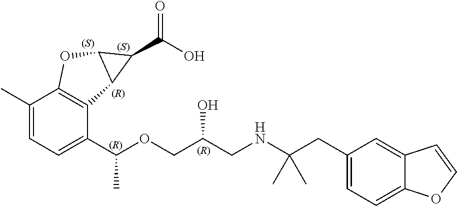 | 42 | ¹H NMR (CD3OD, 300 MHz) δ 7.77-7.76 (d, 1H, J = 3.0 Hz), 7.56 7.47 (m, 2H), 7.23-7.21 (d, 1H, J = 6.0 Hz), 6.97-6.95 (d, 1H, J = 6.0 Hz), 6.83-6.76 (m, 2H), 5.13-5.11 (m, 1H), 4.59- 4.57 (m, 1H), 4.00- 3.98 (m, 1H), 3.53-3.51 (m, 2H), 3.24-3.23 (m, 1H), 3.06-2.99 (m, 3H), 2.18 (s, 3H), 1.49-1.47 (d, 3H, J = 6.0), 1.32 (s, 6H), 1.11-1.10 (m, 1H); LC-MS (ESI): 480.2 [M+ 1]: HPLC (220 nm): 97.5%. |
| 8-8 | 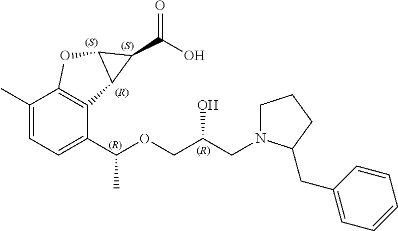 | 489 | ¹H NMR (CD3OD, 300 MHz) δ 7.38-7.19 (m, 5H), 6.97 (d, J = 7.7 Hz, 1H), 6.78 (d, J = 7.7 Hz, 1H), 5.11 (d, J = 5.5 Hz, 1H), 4.59 (q, J = 6.4 Hz, 1H), 4.21-4.06 (m, 1H), 3.92-3.70 (m, 2H), 3.52-3.49 (m, 1H), 3.46-3.32 (m, 5H), 3.25 (m, 1H), 3.20-3.06 (m, 1H), 2.95-2.80 (m, 1H), 2.18 (s, 1H), 2.04 (m, 3H), 1.95 1.67 (m, 1H), 1.53 (d, J = 6.4 Hz, 3H), 1.48 (d, J = 8.1 Hz, 1H), 1.29 (s, 1H), 1.09 (m, 1H): LC-MS (ESI): 452.2 [M + 1]: HPLC (220 nm): 95.3%. |
| 8-9 | 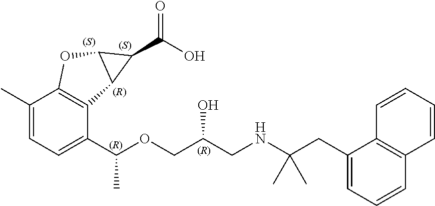 | 119 | ¹H NMR (CD3OD, 300 MHz) δ 8.18-8.16 (d, J = 8.4 Hz, 1H), 7.91-7.83 (m, 2H), 7.59-7.43 (m, 4H), 6.94-6.75 (m, 2H), 5.02-5.00 (m, 1H), 4.69-4.63 (m, 1H), 3.53-3.37 (m, 5H), 3.11-3.03 (m, 1H), 2.14 (s, 3H), 1.98 (s, 1H), 1.53-1.51 (d, J = 6.6 Hz, 3H), 1.20 (s, 6H), 1.03- .02 (m, 1H); LC-MS (ESI): 490.2 [M + 1]; HPLC: 96% |
| 8-10 | 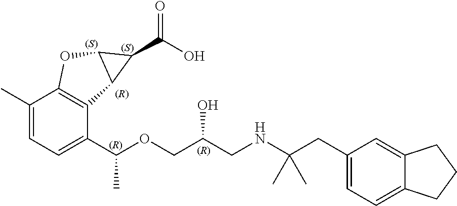 | 15 | ¹H NMR (CD3OD, 300 MHz) δ 7.21-7.19 (d, 1H, J = 6.0 Hz), 7.05 (s, 1H), 6.94-6.88 (m, 2H), 6.73- 6.71 (d, 1H, J = 6.0 Hz), 4.98-4.97 (m, 1H), 4.57- 4.55 (m, 1H), 3.72 (m, 1H), 3.31-3.28 (m, 1H), 3.18-3.15 (m, 1H), 2.83-2.68 (m, 6H), 2.73-2.70 (m, 3H), 2.15 (s, 3H), 2.09-2.03 (m, 3H), 1.50- 1.48 (d, 3H, J = 6.0 Hz), 1.18 (s, 6H), 1.13-1.12 (m, 1H); LC-MS (ESI): 480.2 [M + 1]; HPLC (220 nm): 93.9%. |
| 8-11 | 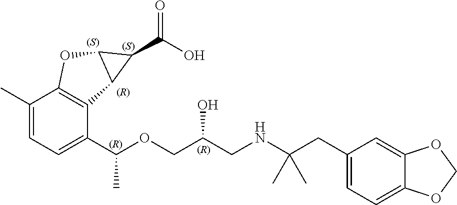 | 50 | ¹H NMR (CD3OD, 300 MHz) δ 6.94-6.91 (d, J = 9.0 Hz, 2H), 6.78-6.67 (m, 4H), 5.93 (s, 2H), 4.99-4.98 (d, J = 3.0 Hz, 1H), 4.63-4.61 (d, J = 6.0 Hz, 1H), 3.86 (m, 1H), 3.34 (m, 1H), 2.87- 2.67 (m, 4H), 2.17 (s, 3H), 1-53-1.51 (d, J = 6.0 Hz, 3H), 1.14 (s, 6H), 1.01-1.00 (m, 1H): LC-MS (ESI): 483.2 [M + 1]; HPLC (220 nm): 95.1%. |
| 8-12 | 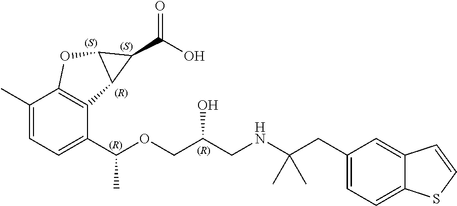 | 10 | ¹H NMR (CD3OD, 300 MHz) δ 7.89-7.86 (d, J = 9.0 Hz, 1H), 7.74 (s, 1H), 7.60-7.58 (d, J = 6.0 Hz, 1H), 7.38-7.36 (d, J = 6.0 Hz, 1H), 7.28-7.25 (d, J = 9.0 Hz, 1H), 6.92-6.90 (d, J = 6.0 Hz, 1H), 6.74-6.72 (d, J = 6.0 Hz, 1H), 5.02-4.97 (m, 1H), 4.69-4.60 (m, 1H), 3.94 (s, 1H), 3.32-3.31 (m, 1H), 3.05-2.98 (m, 4H), 2.01 (s, 3H), 1.49-1.46 (d, J = 9.0 Hz, 3H), 1.29 (s, 6H). 0.98-0.97 (m, 1H): LC-MS (ESI): 495.2 [M + 1]; HPLC (220 nm): 96.8%. |
| 8-13 | 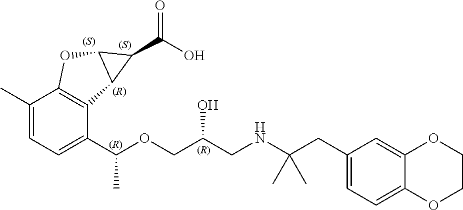 | 22 | ¹H NMR (CD3OD, 300 MHz) δ 6.82-6.80 (d, J = 6.0 Hz, 1H), 6.70-6.56 (m, 4H), 4.88-4.87 (m, 1H), 4.57-4.55 (m, 1H), 4.12 (s, 4H), 3.82 (m, 1H), 3.41-3.38 (m, 1H), 3.08-3.02 (m, 1H), 2.83-2.69 (m, 3H), 2.05 (s, 3H), 1.42-1.40 (d J = 6.0 Hz, 3H), 1.16 (s, 6H), 0.89-0.87 (m, 1H); LC-MS (ESI): 497.2 [M + 1]; HPLC (220 nm): 99.2%. |
| 8-14 | 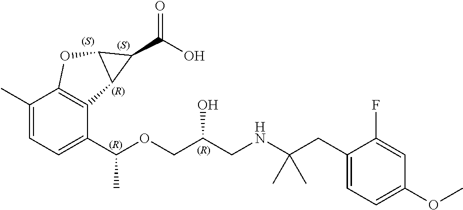 | 30 | ¹H NMR (CD3OD, 300 MHz) δ 7.22-7.16 (t, 1H), 6.96-6.94 (d, J = 6.0 Hz, 1H), 6.78-6.72 (m, 3H), 5.06-5.04 (m, 1H), 4.62-4.60 (m, 1H), 4.01-3.98 (m, 1H), 3.80 (s, 3H), 3.41-3.48 (m, 3H), 3.21-3.24 (m, 2H), 3.08-2.93 (m, 4H), 2.16 (s, 3H), 1.53-1.51 (s, J = 6.0 Hz, 3H), 1.29 (s, 6H), 0.91-0.89 (m, 1H); LC-MS (ESI): 487.2 [M + 1]; HPLC (220 nm): 97.5%. |
| 8-15 | 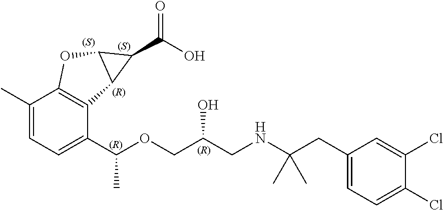 | 14 | ¹H NMR (CD3OD, 300 MHz) δ 7.41-7.39 (t, 2H), 7.23-7.21 (d, 1H, J = 6.0 Hz), 6.88-6.86 (d, 1H, J = 6.0 Hz), 6.74-6.72 (d, 1H, J = 6.0 Hz), 5.06-5.04 (m, 1H), 4.63-4.60 (m, 1H), 3.83-3.81 (m, 1H), 3.43-3.41 (s, 1H), 2.84-2.61 (m, 5H), 2.15 (s, 3H), 1.52-1.50 (d, 3H, J = 6.0 Hz), 1.11 (s, 6H), 0.91- 0.89 (m, 1H); LC-MS (ESI): 508.2 [M + 1]; HPLC (220 nm): 98.4%. |
| 8-16 | 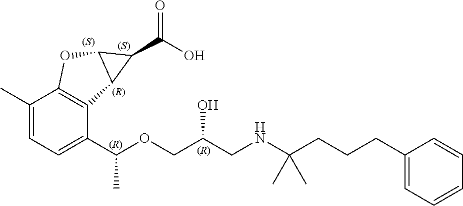 | 29 | ¹H NMR (CD3OD, 300 MHz) δ 7.31-7.08 (m, 5H), 6.97 (d, J = 7.6 Hz, 1H), 6.82-6.74 (m, 1H), 5.10 (d, J = 4.7 Hz, 1H), 4.60 (q, J = 6.4 Hz, 1H), 4.00-3.87 (m, 1H), 3.54-3.47 (m, 1H), 3.47-3.38 (m, 1H), 3.29- 3.22 (m, 1H), 3.06-2.98 (m, 1H), 2.85-2.74 (m, 1H), 2.72-2.60 (m, 2H), 2.19 (s, 3H), 1.74-1.59 (m, 3H), 1.53 (d, J = 6.4 Hz, 3H), 1.31 (s, 6H), 1.10 (m, 1H), 0.95-0.87 (m, 1H); LC-MS (ESI): 468.2 [M + 1]; HPLC (220 nm): 95.0%. |
| 8-17 | 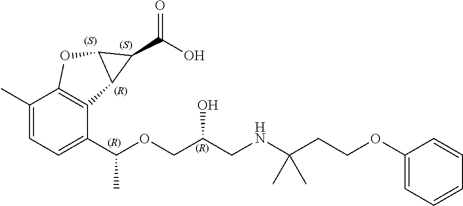 | 90 | ¹H NMR (CD3OD, 300 MHz) δ 7.32-7.27 (m, 2H), 7.01-6.91 (m, 4H), 6.77-6.75 (d, J = 6.0 Hz, 1H), 5.01-4.99 (m, 1H), 4.67-4.65 (m, 1H), 4.20 (m, 2H), 3.96 (m, 1H), 3.42-3.47 (m, 1H), 3.13-3.10 (m, 2H), 2.90-2.87 (m, 1H), 2.16 (m, 5H), 1.56-1.53 (d, J = 9.0 Hz, 3H), 1.41-1.40 (d, 6H), 1.01-1.00 (m, 1H); LC-MS (ESI): 469.2 [M + 1]; HPLC (220 nm): 95.8%. |
| 8-18 | 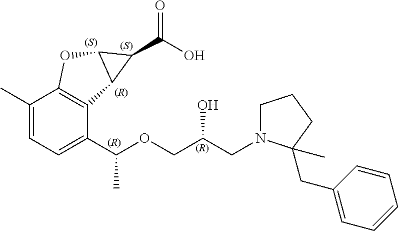 | 1333 | ¹H NMR (CD3OD, 300 MHz) δ 7.32-7.30 (m, 5H), 6.97- 6.94 (m, 1H), 6.80-6.78 (m, 1H), 5.13-5.12 (m, 1H), 4.65-4.62 (m, 1H), 4.20-4.17 (m, 1H), 3.53-3.45 (m, 2H), 3.20-3.17 (m, 1H), 2.94-2.89 (m, 3H), 2.18- 2.04 (m, 7H), 2.94-2.87 (m, 2H), 1.55-1.52 (d, J = 9.0 Hz, 3H), 1.37-1.32 (m, 6H), 1.06-1.05 (m, 1H); LC-MS (ESI): 465.2 [M + 1]; HPLC (220 nm): 89.4%. |
| 8-19 | 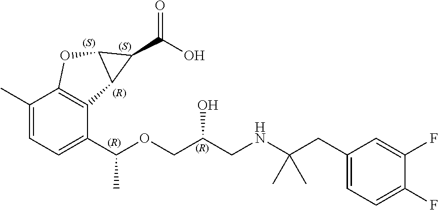 | 138 | ¹H NMR (CD3OD, 300 MHz) δ 7.23-7.20 (m, 2H), 7.13-7.05 (m, 1H), 6.90-6.88 (d, J = 6.0 Hz, 1H), 6.76-6.74 (d, J = 6.0 Hz, 1H), 4.99-4.98 (m, 1H), 4.65-4.63 (m, 1H), 3.91 (m, 1H), 3.43 (m, 1H), 3.09- 3.06 (m, 1H), 2.86-2.84 (m, 3H), 2.15 (s, 3H), 1.54- 1.52 (d, J = 6.0 Hz, 3H), 1.30 (s, 6H), 0.99-0.98 (m, 1H); LC-MS (ESI): 475.2 [M + 1]; HPLC (220 nm): 98.7%. |
| 8-20 | 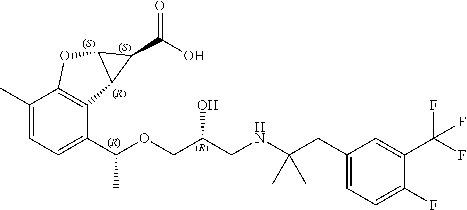 | 64 | ¹H NMR (CD3OD, 300 MHz) δ 7.66 (s, 1H), 7.60- 7.57 (d, J = 9.0 Hz, 1H), 7.52-7.49 (d, J = 9.0 Hz, 1H), 6.93-6.90 (d, J = 9.0 Hz, 1H), 6.77-6.74 (d, J = 9.0 Hz, 1H), 4.99-4.98 (m, 1H), 4.67-4.64 (m, 1H), 3.94 (m, 1H), 3.48-3.46 (m, 1H), 3.22-2.92 (m, 4H), 2.14 (s, 3H), 1.54-1.52 (d, J = 6.0 Hz, 3H), 1.26 (s, 6H), 0.99-0.98 (m, 1H); LC-MS (ESI): 541.2 [M + 1]; HPLC (220 nm): 97.4%. |
| 8-21 | 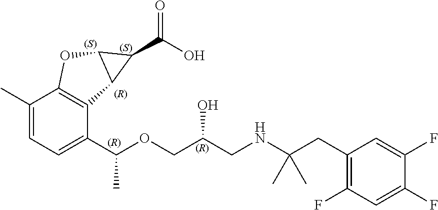 | 99 | ¹H NMR (CD3OD, 300 MHz) δ 7.33-7.22 (m, 2H), 6.98-6.96 (d, J = 6.0 Hz, 1H), 6.78-6.76 (d, J = 6.0 Hz, 1H), 5.05-5.04 (d, J = 3.0 Hz, 1H), 4.68-4.61 (m, 1H), 4.00 (m, 1H), 3.96-3.94 (m, 3H), 3.25- 3.24 (m, 1H), 3.04-2.97 (m, 3H), 2.16 (s, 3H), 1.55-1.53 (d, J = 6.0 Hz, 3H), 1.31 (s, 6H), 0.91- 0.90 (m, 1H); LC-MS (ESI): 494.2 [M + 1]; HPLC (220 nm): 97.8%. |
| 8-22 | 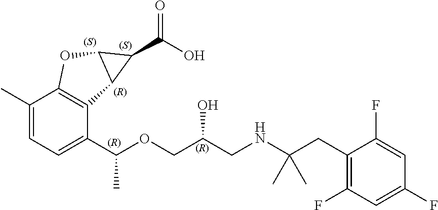 | 210 | ¹H NMR (CD3OD, 300 MHz) δ 6.96-6.90 (m, 3H), 6.77-6.74 (d, J = 9.0 Hz, 1H), 4.99-4.97 (d, J = 6.0 Hz, 1H), 4.66-4.64 (m, 1H), 3.96-3.95 (m, 1H), 3.45- 3.42 (m, 1H), 3.25-3.24 (m, 1H), 3.06-2.97 (m, 3H), 2.14 (s, 3H), 1.55-1.53 (d, J = 6.0 Hz, 3H), 1.27 (s, 6H), 0.99-0.98 (m, 1H); LC-MS (ESI): 494.2 [M + 1]; HPLC (220 nm): 95.4%. |
| 8-23 | 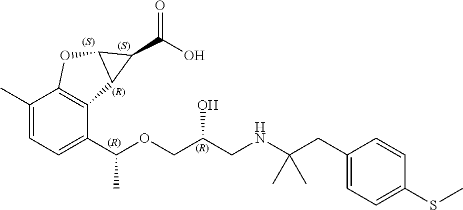 | 40 | ¹H NMR (CD3OD, 300 MHz) δ 7.25-7.16 (m, 2H), 6.93-6.73 (m, 2H), 4.99-4.97 (m, 1H), 4.68-4.61 (m, 1H), 3.97-3.93 (m, 1H), 3.28-3.15 (m, 3H), 2.99-2.92 (m, 1H), 2.90 (s, 2H), 2.47 (s, 3H), 1.92 (s, 1H), 1.53-1.51 (d, J = 6.3 Hz, 3H), 1.27 (s, 6H), 1.00-0.98 (m, 1H); LC-MS (ESI): 486.2 [M + 1]; HPLC: 98%. |
| 8-24 | 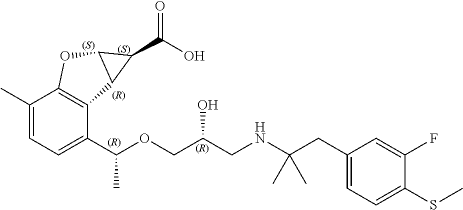 | 21 | ¹H NMR (CD3OD, 300 MHz) δ 7.30 (t, J = 8.0 Hz, 1H), 7.11-6.99 (m, 2H), 6.95 (d, J = 7.7 Hz, 1H), 6.77 (d, J = 7.7 Hz, 1H), 5.05 (dd, J = 5.4, 1.0 Hz, 1H), 4.63 (q, J = 6.4 Hz, 1H), 3.99 (m, 1H), 3.52- 3.48 (m, 1H), 3.45-3.42 (m, 1H), 3.36-3.34 (m, 1H), 3.25-3.21 (m, 1H), 3.06-2.91 (m, 3H), 2.46 (s, 3H), 2.16 (s, 3H), 1.52 (d, J = 6.4 Hz, 3H), 1.27 (s, 6H), 0.93-0.88 (m, 1H); LC-MS (ESI): 504.1 [M + 1]; HPLC (220 nm): 96.0%. |
| 8-25 | 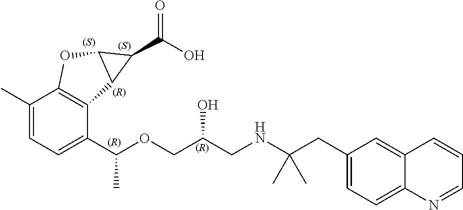 | 460 | ¹H NMR (CD3OD, 300 MHz) δ 8.87-8.86 (d, J = 3.0 Hz, 1H), 8.37-8.34 (d, J = 9.0 Hz, 1H), 8.06- 8.03 (d, J = 9.0 Hz, 1H), 7.89 (s, 1H), 7.16-7.74 (d, J = 6.0 Hz, 1H), 7.56-7.54 (d, J = 6.0 Hz, 1H), 6.95-6.93 (d, J = 6.0 Hz, 1H), 6.78-6.76 (d, J = 6.0 Hz, 1H), 5.02-5.00 (d, J = 6.0 Hz, 1H), 4.68- 4.66 (m, 1H), 3.98 (m, 1H), 3.50-3.47 (m, 1H), 3.15-2.98 (m, 3H), 2.16 (s, 3H), 1.50-1.48 (d, J = 6.0 Hz, 3H), 1.31 (s, 6H), 1.01-1.00 (m, 1H); LC-MS (ESI): 491.3 [M + 1]; HPLC (220 nm): 95.0%. |
| 8-26 | 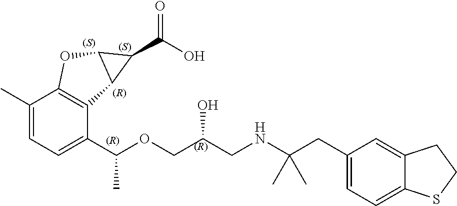 | 21 | ¹H NMR (CD3OD, 300 MHz) δ 7.21-7.08 (m, 2H), 6.97-6.89 (m, 2H), 6.74-6.72 (d, J = 6.0 Hz, 1H), 4.98-4.96 (m, 1H), 4.60-4.58 (m, 1H), 3.88 (m, 1H), 3.45-3.42 (m, 2H), 3.27-3.24 (m, 3H), 3.03-2.98 (m, 1H), 2.81-2.78 (m, 3H), 2.14 (s, 3H), 1.52-1.50 (d, J = 6.0 Hz, 3H), 1.24 (s, 6H), 1.10-1.09 (m, 1H); LC-MS (ESI): 498.2 [M + 1]; HPLC (220 nm): 97.7%. |
| 8-27 | 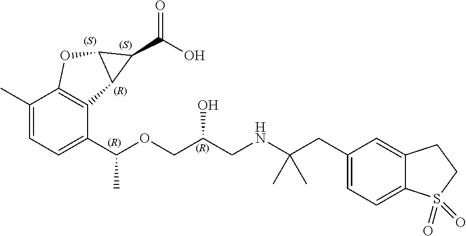 | >10000 | ¹H NMR (CD3OD, 300 MHz) δ 7.70-7.68 (d, J = 6.0 Hz, 1H), 7.46-7.44 (m, 2H), 6.97-6.95 (d, J = 6.0 Hz, 1H), 6.82-6.80 (d, J = 6.0 Hz, 1H), 5.04- 5.02 (m, 1H), 4.70-4.66 (m, 2H), 4.00 (m, 1H), 3.57-3.53 (m, 4H), 3.13-3.02 (m, 5H), 2.18 (s, 3H), 2.06-2.03 (m, 3H), 1.54-1.52 (d, J = 6.0 Hz, 3H), 1.33 (s, 6H), 1.06-1.05 (m, 1H); LC-MS (ESI): 530.2 [M + 1]; HPLC (220 nm): 97.3%. |
| 8-28 | 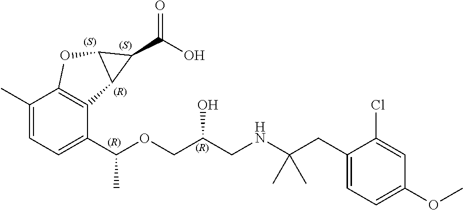 | 22 | ¹H NMR (CD3OD, 300 MHz) δ 7.26-7.23 (d, J = 9.0 Hz, 1H), 6.99-6.98 (d, J = 3.0 Hz, 1H), 6.89- 6.85 (m, 2H), 6.75-6.72 (d, J = 9.0 Hz, 1H), 4.98- 4.94 (m, 1H), 4.67-4.60 (m, 1H), 3.93 (m, 1H), 3.79 (s, 3H), 3.47-3.45 (m, 1H), 3.06-2.86 (m, 4H), 2.14 (s, 3H), 1.54-1.52 (d, J = 6.0 Hz, 3H), 1.24 (s, 6H), 1.00-0.99 (m, 1H); LC-MS (ESI): 504.2 [M + 1]; HPLC (220 nm): 96.6%. |
| 8-29 | 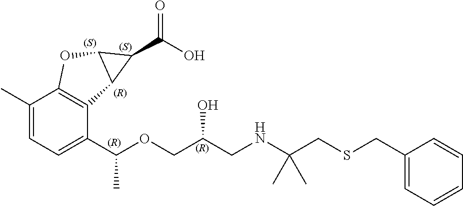 | 500 | ¹H NMR (CD3OD, 300 MHz) δ 7.37-7.22 (m, 5H), 7.00-6.97 (d, J = 9.0 Hz, 1H), 6.82-6.79 (d, J = 9.0 Hz, 1H), 5.10 (m, 1H), 4.65-4.62 (m, 1H), 3.93 (m, 1H), 3.84 (s, 1H), 3.51-3.45 (m, 2H), 2.97-2.77 (m, 4H), 2.20 (s, 3H), 1.58-1.55 (d, J = 9.0 Hz, 3H), 1.40 (s, 6H), 1.11-1.10 (m, 1H); LC-MS (ESI): 486.2 [M + 1]; HPLC (220 nm): 90.6%. |
| 8-30 | 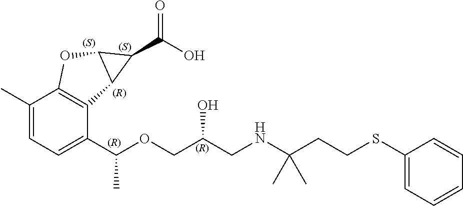 | 50 | ¹H NMR (CD3OD, 300 MHz) δ 7.40-7.20 (m, 5H), 6.96-6.93 (d, J = 9.0 Hz, 1H), 6.76-6.73 (d, J = 9.0 Hz, 1H), 4.99 (m, 1H), 4.65-4.63 (m, 1H), 3.88 (m, 1H), 3.45 (m, 1H), 3.02-2.97 (m, 4H), 2.87-2.77 (m, 1H), 2.15 (s, 3H), 1.96-1.91 (m, 2H), 1.53-1.51 (d, J = 6.0 Hz, 3H), 1.34 (s, 6H), 0.99-0.98 (m, 1H); LC-MS (ESI): 486.2 [M + 1]; HPLC (220 nm): 95.1%. |
| 8-31 | 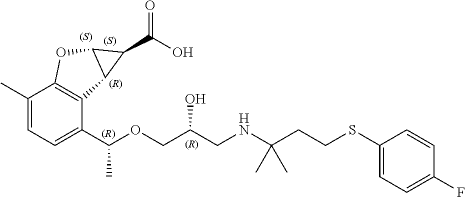 | 65 | ¹H NMR (CD3OD, 300 MHz) δ 7.46-7.41 (m, 2H), 7.08-7.02 (m, 2H), 6.86-6.83 (d, 1H, J = 9.0 Hz), 6.74-6.71 (d, 1H, J = 9.0 Hz), 4.99 (m, 1H), 4.65- 4.63 (m, 1H), 3.84-3.82 (m, 1H), 3.35-3.26 (m, 2H), 2.84-2.76 (m, 4H), 2.67-2.63 (m, 1H), 2.15 (s, 3H), 1.86-1.84 (m, 2H), 1.53-1.51 (d, 3H, J = 6.0 Hz), 1.35 (s, 6H), 0.99-0.98 (m, 1H); LC-MS (ESI): 504.2 [M + 1]; HPLC (220 nm): 96.6%. |
| 8-32 | 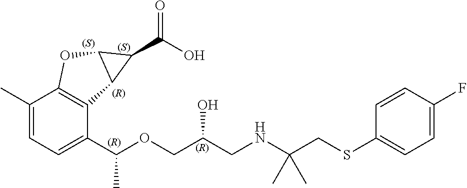 | 1410 | ¹H NMR (CD3OD, 300 MHz) δ 7.57-7.52 (m, 2H), 7.14-7.08 (m, 2H), 6.99-6.96 (d, J = 9.0 Hz, 1H), 6.80-6.77 (d, J = 9.0 Hz, 1H), 5.08 (m, 1H), 4.65- 4.63 (m, 1H), 3.97 (m, 1H), 3.47 (m, 1H), 3.20- 3.10 (m, 2H), 2.96-2.93 (m, 1H), 2.19 (s, 3H), 1.56- 1.53 (d, J = 9.0 Hz, 3H), 1.36 (s, 6H), 1.07-1.06 (m, 1H); LC-MS (ESI): 490.2 [M + 1]; HPLC (220 nm): 89.3%. |
| 8-33 | 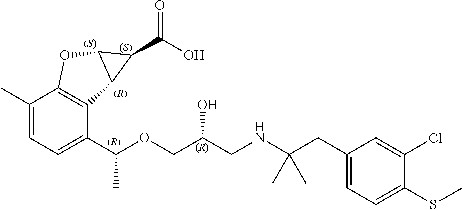 | 30 | ¹H NMR (CD3OD, 300 MHz) δ 7.30-7.19 (m, 3H), 6.94- 6.91 (d, J = 9.0 Hz, 1H), 6.77-6.74 (d, J = 9.0 Hz, 1H), 5.00 (m, 1H), 4.67-4.65 (m, 1H), 3.96 (m, 1H), 3.49-3.45 (m, 1H), 3.24-3.21 (m, 1H), 3.03-2.91 (m, 3H), 2.48 (s, 3H), 2.14 (s, 3H), 1.53-1.51 (d, J = 6.0 Hz, 3H), 1.29 (s, 6H), 0.99 (m, 1H); LC-MS (ESI): 520.2 [M + 1]; HPLC (220 nm): 97.0%. |
| 8-34 | 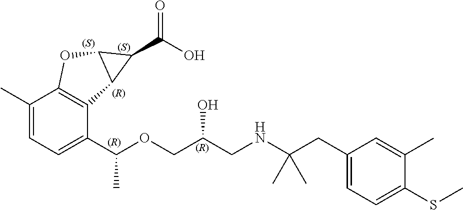 | 82 | ¹H NMR (CD3OD, 300 MHz) δ 7.21-7.06 (m, 3H), 6.95- 6.92 (d, J = 9.0 Hz, 1H), 6.78-6.75 (d, J = 9.0 Hz, 1H), 5.01 (m, 1H), 4.67-4.65 (m, 1H), 3.95 (m, 1H), 3.49-3.45 (m, 1H), 3.23-3.19 (m, 1H), 3.03-2.91 (m, 3H), 2.47 (s, 3H), 2.31 (s, 3H), 2.16 (s, 3H), 1.54-1.51 (d, J = 9.0 Hz, 3H), 1.26 (s, 6H), 1.01-1.00 (m, 1H); LC-MS (ESI): 500.2 [M + 1]; HPLC(220 nm): 92.7%. |

### Example 9

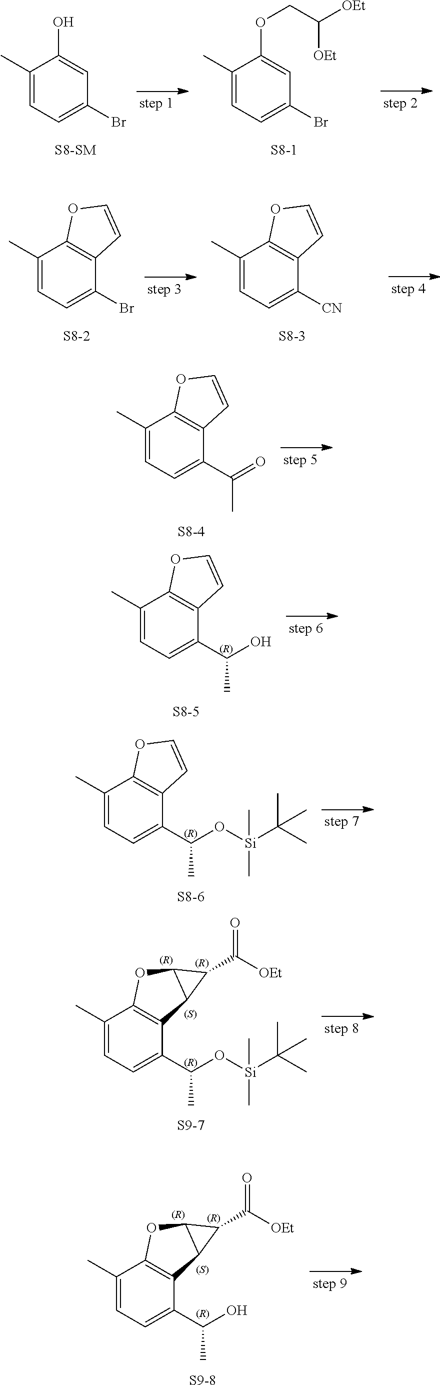

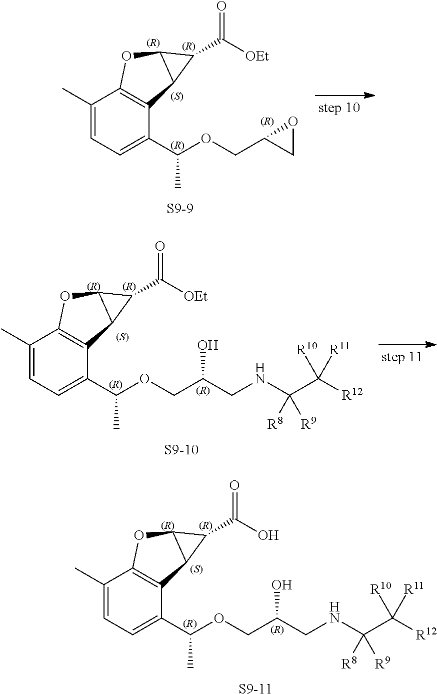

### Experimental Procedure

The experimental procedure in EXAMPLE 9 was the same as that in EXAMPLE 8 except (R,R)-(+)-2,2-Isopropylidenebis(4-tert-butyl-2-oxazoline) was used in step 7.

The following compounds in Table 9 were made according to EXAMPLE 9.

TABLE 9

| | | FLIPR | |
|---|---|---|---|
| Ex- | | ASSAY | |
| am- | | IC₅₀ | |
| ple | Structure | (nM) | Analysis Data |
| 9-1 | 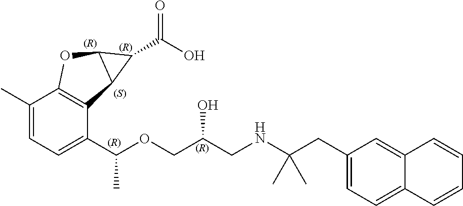 | 22 | ¹H NMR (CD3OD, 300 MHz) δ 7.87-7.84 (m, 3H), 7.77 (s, 1H), 7.50-7.38 (m, 3H), 6.96-6.94 (d, 1H), 6.79-6.76 (d, J = 9.0 Hz, 1H), 5.12-5.10 (d, J = 6.0 Hz, 1H), 4.59-4.57 (m, 1H), 4.00 (m, 1H), 3.50-3.30 (m, 4H), 3.15-3.04 (m, 3H), 2.18 (s, 3H), 1.48-1.45 (d, J = 9.0 Hz, 3H), 1.36 (s, 6H), 1.10-1.09 (m, 1H); LC-MS (ESI): 490.3 [M + 1]: HPI.C (220 nm): 98.1%. |
| 9-2 | 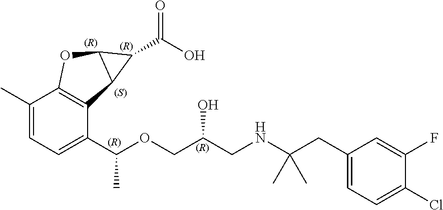 | 78 | ¹H NMR (CD3OD, 300 MHz) δ 7.45-7.43 (t, J = 7.8 Hz, 1H), 7.24 (d, 1H), 7.15 (d, 1H), 7.86-7.92 (d, 1H), 6.65-6.63 (d, 1H), 5.03-5.00 (d, 1H), 4.59-4.57 (m, 1H), 4.02 (m, 1H), 3.32-3.18 (m, 3H), 3.00-2.92 (m, 3H), 2.16 (s, 3H), 1.50-1.48 (m, 3H), 1.29 (s, 6H), 0.89-0.88 (m, 1H); LC-MS (ESI): 492.2 [M + 1]; HPLC (220 nm): 97.3%. |
| 9-3 | 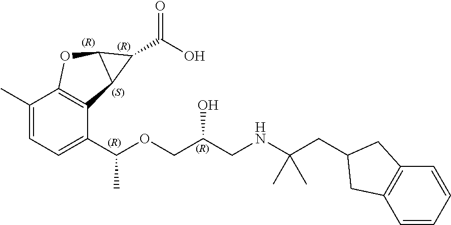 | 46 | ¹H NMR (CD3OD, 300 MHz) δ 7.15-7.05 (m, 4H), 6.90-6.87 (d, J = 9.0 Hz, 1H), 6.75-6.72 (d, J = 9.0 Hz, 1H),. 4.97-4.95 (m, 1H), 4.65-4.62 (m, 1H), 3.91-3.89 (m, 1H), 3.41-3.33 (m, 2H), 3.16-3.02 (m, 3H), 2.89-2.61 (m, 4H), 2.14 (s, 3H), 1.89 (d, 2H), 1.55-1.52 (d, J = 9.0 Hz, 3H), 1.33 (s, 6H), 1.00-0.98 (m, 1H); LC-MS (ESI) 480.3 [M + 1]; HPLC (220 nm): 95.8%. |
| 9-4 | 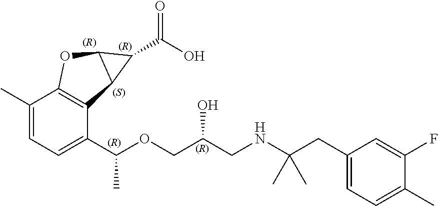 | 68 | ¹H NMR (DMSO-d₆, 300 MHz) δ 7.46-7.42 (m, 1H), 7.01-6.96 (m, 3H), 6.83-6.80 (m, 1H), 5.14- 5.10 (m, 1H), 4.71-4.69 (q, J = 6.0 Hz, 1H), 4.03- 3.99 (m, 1H), 3.49-3.40 (m, 3H), 3.10-2.94 (m, 3H), 2.24 (s, 3H), 2.18 (s, 3H), 1.50-1.48 (d, J = 6.0 Hz, 3H), 1.30 (s, 6H), 1.17-1.08 (m, 1H), LC-MS (ESI): 472.2 [M + 1]; HPLC (220 nm): 98.7% |
| 9-5 | 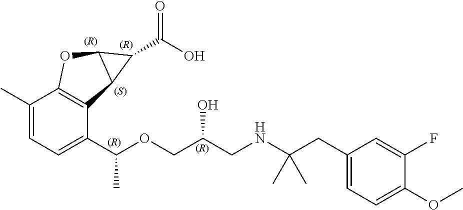 | 224 | ¹H NMR (DMSO-d₆, 300 MHz) δ 7.10-6.91 (m, 4H), 6.83-6.80 (m, 1H), 5.14-5.10 (m, 1H), 4.71- 4.69 (q, J = 6.0 Hz, 1H), 4.03-4.01 (m, 1H), 3.82 (s, 3H), 3.49-3.40 (m, 3H), 3.29 (s, 3H),. 3.07-2.91 (m, 3H), 2.17 (s, 3H), 1.48-1.46 (d, J = 6.0 Hz, 3H), 1.30 (s, 6H), 1.17-1.08 (m, 1H), LC-MS(ESI) 488.2 [M + 1]; HPLC (220 nm): 99.3%. |
| 9-6 | 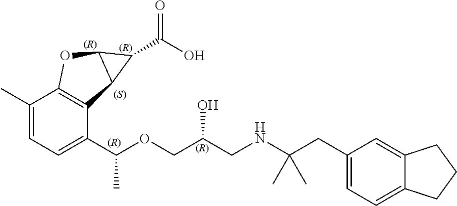 | 53 | ¹H NMR (CD₃OD, 300 MHz) δ 7.19-7.17 (d, J = 6.0 Hz, 1H), 7.11 (s, 1H), 7.00-6.98 (d, J = 6.0 Hz, 1H), 6.81-6.79 (d, J = 9.0 Hz, 1H),. 5.12 (m, 1H), 4.69-4.67 (m, 1H), 4.01 (m, 1H), 3.41-3.33 (m, 2H), 3.16-3.02 (m, 3H), 2.89-2.61 (m, 4H), 2.18 (s, 3H), 2.12-2.04 (m, 2H), 1.45-1.43 (d, J = 6.0 Hz, 3H), 1.30 (s,6H), 1.08-1.07 (m, 1H): LC-MS (ESI) 480.3 [M + 1]: HPLC (220 nm): 97.5%. |
| 9-7 | 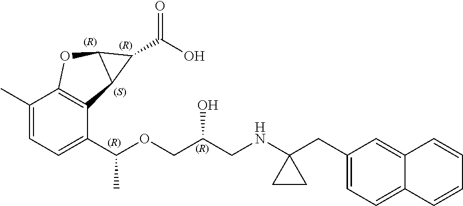 | 187 | ¹H NMR (CD3OD, 300 MHz,) δ 7.99-7.64 (m, 4H), 7.56-7.47 (m, 3H), 6.97 (d, J = 8.1, 1H), 6.75 (d, J = 8.1, 1H), 5.13 (dd, J = 5.4, 1.0 Hz, 1H), 4.68-4.61 (m, 1H), 4.00-3.97 (m, 1H), 3.43-3.29 (m, 4H), 3.25-3.10 (m, 3H), 2.18 (s, 3H), 1.39 (d, J = 6.3, 3H), 1.12-1.07 (m, 3H), 0.99-0.99 (m, 2H); LC-MS (ESI): 488.2 [M + 1]; HPLC (220 nm): 99.2%. |
| 9-8 | 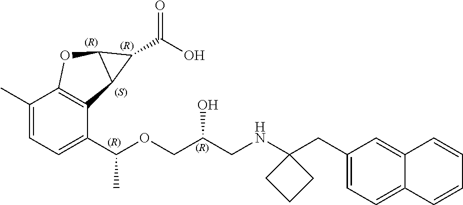 | >10000 | ¹H NMR (CD₃OD, 300 MHz,) δ 7.89-7.84 (m, 4H), 7.54-7.47 (m, 3H), 6.97 (d, J = 8.1, 1H), 6.76 (d, J = 8.1, 1H), 5.13 (dd, J = 5.4, 1.0 Hz, 1H), 4.68-4.60 (m, 1H), 4.05-3.99 (m, 1H), 3.56-3.37 (m, 3H), 3.27-3.16 (m, 3H), 2.42-2.34 (m, 4H), 2.18 (s, 3H), 1.44 (d, J = 6.3, 3H), 1.10-1.01 (m, 2H), 0.94-0.83 (m, 2H); LC-MS (ESI): 502.3 [M + 1]: HPLC (220 nm): 93.7% |
| 9-9 | 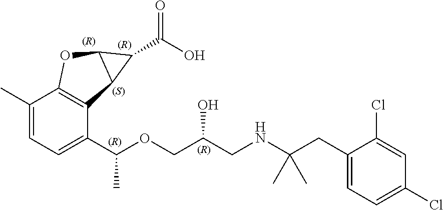 | 77 | ¹H NMR (DMSO-d₆, 300 MHz) δ 7.76-7.74 (m, 1H), 7.45-7.36 (m, 1H), 6.93-6.91 (m, 1H), 6.71- 6.69 (m, 1H), 5.01-4.99 (m, qH), 4.58-4.56 (q, J = 6.0 Hz, 1H), 3.87 (br s, qH), 3.19-3.15 (m, 2H), 3.05-2.95 (m, 2H), 2.78-2.73 (m, 2H),. 2.12 (s, 3H), 1.39-1.37 (d, J = 6.0 Hz, 3H), 1.09 (s, 6H), 0.87- 0.86 (m, 1H), LC-MS (ESI): 508.2 [M + 1]: HPLC (220 nm): 97.0%. |
| 9-10 | 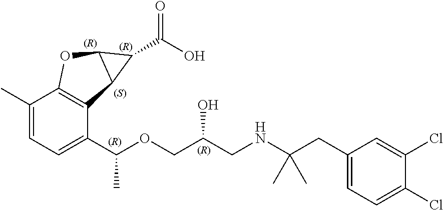 | 42 | ¹H NMR (300 MHz, DMSO-d₆) δ 7.52-7.49 (m, 1H), 7.26-7.24 (m, 1H), 7.00-6.97 (m, 1H), 6.76- 6.70 (m, 1H), 5.02-5.00 (m, 1H), 4.66-4.64 (q, J = 6.0 Hz, 1H), 4.03-4.01 (m, 1H), 3.47-3.42 (m, 3H), 3.28-3.26 (m, 1H), 3.03-2.98 (m, 2H), 2.16 (s, 3H), 1.49-1.47 (d, J = 6.0 Hz, 3H), 1.30 (s, 6H), 1.01-1.00 (m, 1H); LC-MS (ESI): 508. 2 \|M + 1]: HPLC (220 nm): 98.2%. |

### Example 10

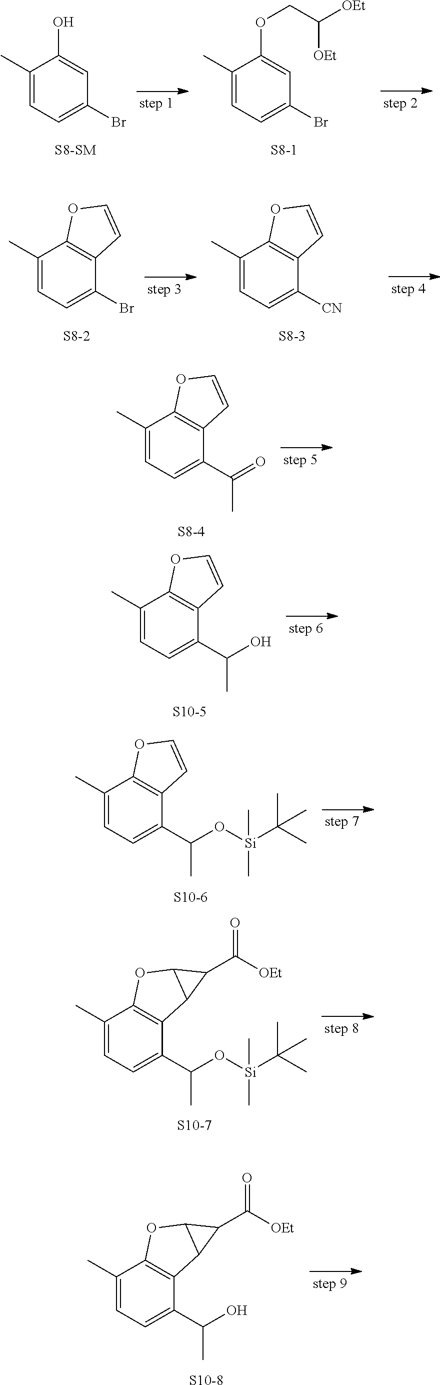

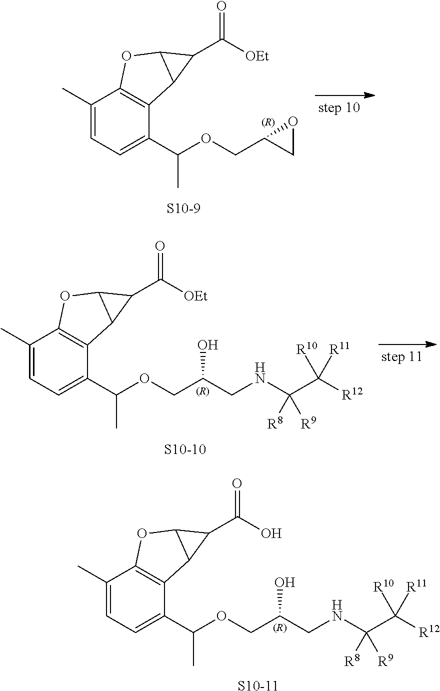

### Experimental Procedure

The experimental procedure in EXAMPLE 10 was the same as that in EXAMPLE 8 except no chiral reagents were used in step 5 and step 7.

The following compounds in Table 10 were made according to EXAMPLE 10.

TABLE 10

| Example | Structure | Analysis Data |
|---|---|---|
| 10-1 | 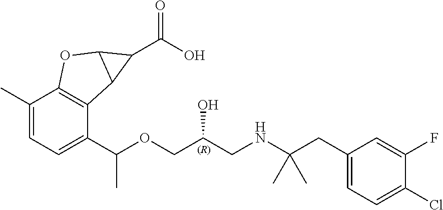 | LC-MS: (M + H)⁺ 492.2 |
| 10-2 | 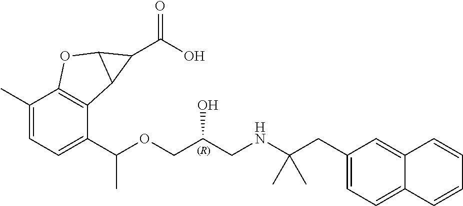 | LC-MS: (M + H)⁺ 490.2 |
| 10-3 | 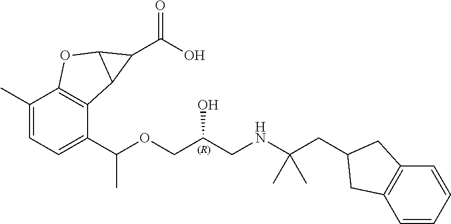 | LC-MS: (M + H)⁺ 480.3 |

### Example 11

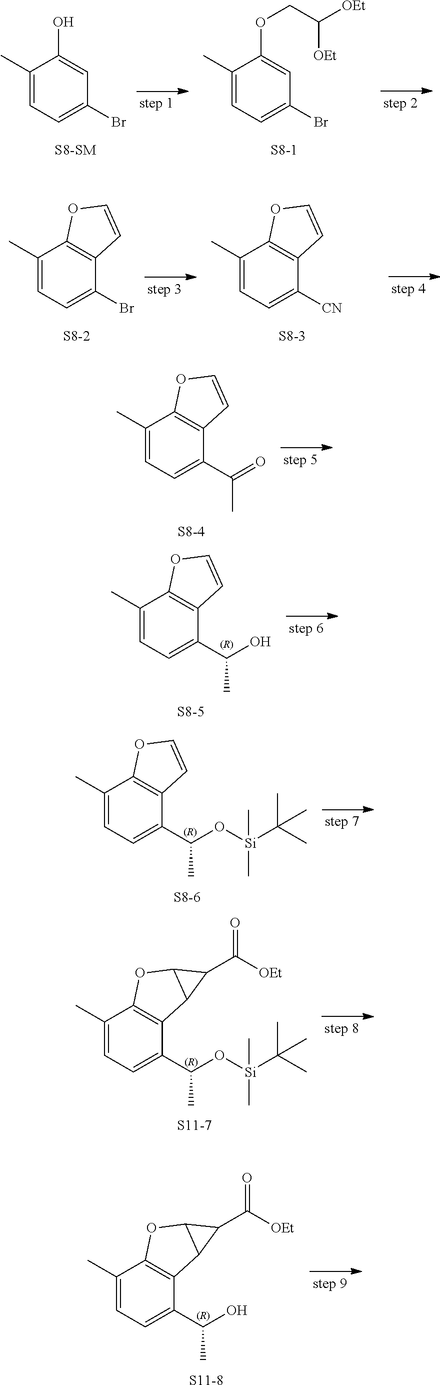

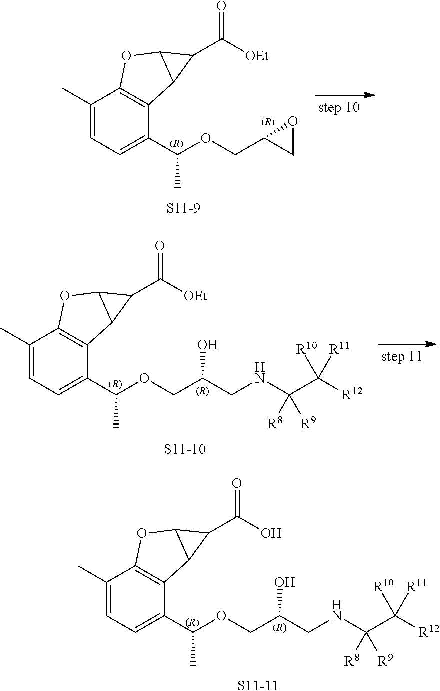

### Experimental Procedure

The experimental procedure in EXAMPLE 11 was the same as that in EXAMPLE 8 except no chiral ligand was used in step 7.

The following compound in Table 11 was made according to EXAMPLE 11.

TABLE 11

| Example | Structure | Analysis Data |
|---|---|---|
| 11-1 | 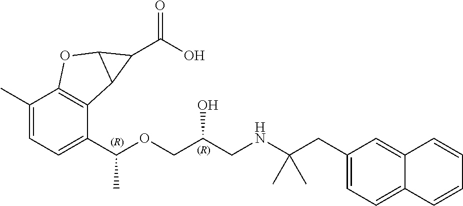 | LC-MS: (M + H)⁺ 490.3 |

### Example 12

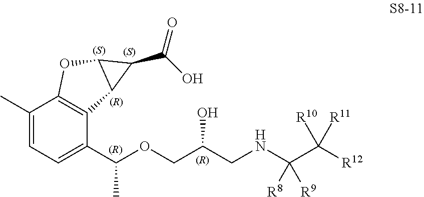

### Experimental Procedure

S8-11 was made according to EXAMPLE 8.

The following compound in Table 12 was made according to EXAMPLE 12.

TABLE 12

| | | FLIPR | |
|---|---|---|---|
| Ex- | | ASSAY | |
| am- | | IC₅₀ | |
| ple | Structure | (nM) | Analysis Data |
| 12-1 | 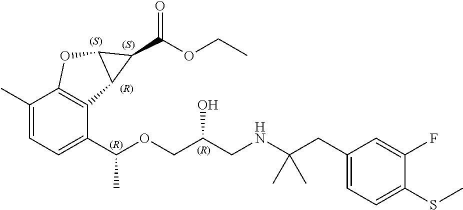 | 130 | ¹H NMR (301 MHz, cd₃od) δ 7.30 (t, J = 7.9 Hz, 1H), 7.10-6.96 (m, 3H), 6.79 (d, J = 7.7 Hz, 1H), 5.12 (dd, J = 5.5, 1.1 Hz, 1H), 4.60 (q, J = 6.4 Hz, 1H), 4.17 (q, J = 7.0 Hz, 2H), 3.99 (dd, J = 9.2, 2.8 Hz, 1H), 3.51 (ddd, J = 14.7, 7.7, 4.0 Hz, 2H), 3.25 (dd, J = 12.2, 2.8 Hz, 1H), 3.14-2.89 (m, 3H), 2.47 (s, 3H), 2.18 (s, 3H), 1.49 (t, J = 7.7 Hz, 3H), 1.35-1.22 (m, 9H), 1.18 (dd, J = 3.1, 1.1 Hz, 1H). LCMS (ESI): 532.3 [M + 1]. HPI.C (220 nm): 97.40%. |

### Example 13

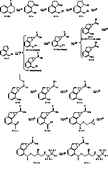

### Experimental Procedure

### Step 1

To a mixture of aluminum chloride (37.0 g, 278 mmol) and sodium chloride (3.7 g, 61.3 mmol) was added chroman-4-one (S13-SM, 6.4 g, 43.2 mmol) at 150° C. After being allowed to stir at 180° C. for 30 mins, the mixture was cooled to 160° C. and poured into ice-cold hydrochloric acid (100 mL) carefully and stirred for 30 mins. The mixture was extracted with methylene chloride and the organic phases were combined and washed with brine. The organic phase was separated, dried with anhydrous Na₂SO₄, filtered and concentrated to give a residue, which was purified by column chromatography to afford S13-1. ¹H NMR (300 MHz, CDCl₃) δ 2.68-2.73 (m, 2H), 3.08-3.12 (m, 2H), 6.72-6.75 (d, J=8.1 Hz, 1H), 6.91-6.93 (d, J=7.2 Hz, 1H), 7.42-7.47 (m, 1H), 9.04 (s, 1H); LC-MS: (M+H)⁺149.

### Step 2

A mixture of S13-1 (10.36 g, 70 mmol), benzyl bromide (12 g, 70 mmol) and potassium carbonate (10.63 g, 77 mmol) in acetone (300 mL) was refluxed overnight. Then the reaction mixture was cooled to room temperature and filtered. The filtrate was concentrated and the residue was purified by column chromatography to afford S13-2. ¹H NMR (300 MHz, CDCl₃) δ 2.65-2.69 (m, 2H), 3.05-3.09 (m, 2H), 5.26 (s, 2H), 6.73-6.76 (d, J=8.4 Hz, 1H), 6.96-6.98 (d, J=7.5 Hz, 1H), 7.24-7.46 (m, 6H); LC-MS: (M+H)⁺239.

### Step 3

To a solution of S13-2 (15.97 g, 67.1 mmol) in methanol (250 mL) was added in portion NaBH₄ (2.04 g, 53.7 mmol). After being allowed to stir for 1 hr, the reaction mixture was filtered and the filtrate was concentrated to provide a residue, which was purified by column chromatography to afford S13-3. ¹H NMR (300 MHz, CDCl₃) δ 1.98-2.09 (m, 1H), 2.40-2.51 (m, 1H), 2.64 (s, 1H), 2.76-2.87 (m, 1H), 3.04-3.14 (m, 1H), 5.12 (s, 2H), 5.49-5.53 (m, 1H), 6.73-6.76 (d, J=7.8 Hz, 1H), 6.84-6.87 (d, J=7.8 Hz, 1H), 7.16-7.42 (m, 1H); LC-MS: (M+Na)⁺263.

### Step 4

To a solution of S13-3 (15.46 g, 64.4 mmol) in toluene (130 mL) was added 4-methylbenzenesulfonic acid (1.1 g, 6.44 mmol). After being allowed to stir for 1 hr at 80° C., the mixture was cooled to room temperature and washed with sat. aq. sodium bicarbonate and brine. The organic layer was separated, dried with anhydrous Na₂SO₄, filtered and concentrated to give a residue, which was purified by column chromatography to afford S13-4. ¹H NMR (300 MHz, CDCl₃) δ 3.43-3.44 (m, 2H), 5.17 (s, 2H), 6.46-6.50 (m, 1H), 6.82-6.88 (m, 1H), 7.10-7.18 (m, 3H), 7.31-7.50 (m, 5H).

### Step 5

To a mixture of S13-4 (1.0 g, 4.5 mmol) and copper bromide (5 mg) in 1,2-dichloroethane (15 mL) was added drop-wise a solution of ethyl diazoacetate (1.79 g, 15.8 mmol) in 1,2-dichloroethane (4 mL) at 80° C. After being allowed to stir for 15 mins, the mixture was cooled to room temperature, concentrated and purified by column chromatography to afford S13-5a and S13-5b. S13-5a (exo-isomer): ¹H NMR (300 MHz, CDCl₃) δ 1.37-1.50 (m, 4H), 2.52-2.57 (m, 1H), 3.17-3.46 (m, 3H), 4.26-4.32 (m, 2H), 5.10 (s, 1H), 6.84-6.93 (m, 2H), 7.17-7.22 (m, 1H), 7.43-7.59 (m, 4H). S13-5b (endo-isomer): ¹H NMR (300 MHz, CDCl₃) δ 0.94-0.99 (m, 3H), 2.03-2.08 (m, 1H), 2.21-2.26 (m, 1H), 3.10-3.38 (m, 3H), 3.82-3.89 (m, 2H), 5.11 (s, 2H), 6.66-6.69 (d, J=8.1 Hz, 1H), 6.75-6.77 (d, J=7.5 Hz, 1H), 7.02-7.08 (m, 1H), 7.29-7.44 (m, 5H).

### Step 6

To a solution of S13-5a (5.0 g, 16.2 mmol) in methanol (20 mL) and THF (20 mL) was added 2 N sodium hydroxide solution (12 mL) and the mixture was stirred at 60° C. for 2 hrs. The organic solvent was removed in vacuo and the aqueous residue was acidized to PH 1-2 with 2 N diluted hydrochloric acid. The mixture was filtered and the cake was washed with water and dried to give S13-6. ¹H NMR (300 MHz, DMSO-d₆) δ 0.99-1.01 (m, 1H), 2.25-2.30 (m, 1H), 2.83-2.85 (m, 1H), 2.96-3.02 (m, 1H), 3.15-3.23 (m, 1H), 5.14 (s, 2H), 6.74-6.83 (m, 2H), 7.02-7.07 (m, 1H), 7.27-7.45 (m, 5H), 12.20 (s, 1H); LC-MS: (M+H)⁺281.

### Step 7

To a solution of S13-6 (78 g, 0.28 mol) in acetone: H₂O=8:5 (1.0 L) was added (R)-1-phenylethylamine (33.7 g, 0.28 mol) at 55° C. After being allowed to stir for 3 hrs at room temperature, the mixture was filtered and the cake was washed with the solvent of acetone:H₂O=8:5, dried, and then dispersed in H₂O, and acidized with hydrochloric acid to PH 2-3. The resulting mixture was stirred for 3 hrs, filtered and the cake was washed with water and dried to give S13-7a. S13-7b was recovered from the mother liquor.

### Step 8

The mixture of S13-7a (7.56 g, 27 mmol), potassium carbonate (3.92 g, 28.35 mmol) and ethyl iodide (4.63 g, 29.7 mmol) in DMF (50 mL) was stirred at room temperature overnight. The mixture was poured into water and extracted with dichloromethane. The combined organics were washed with brine. The organic layer was separated, dried over Na₂SO₄, filtered and concentrated to give a residue, which was purified on silica gel column chromatography (eluted with petroleum ether:ethyl acetate=20:1) to give S13-8a.

### Step 9

A mixture of S13-8a (8.46 g, 27.5 mmol) and 10% Pd/C (0.45 g) in ethanol (110 mL) was stirred overnight under H₂ atmosphere. The reaction mixture was filtered, and the filtrate was concentrated to provide a residue, which was purified on silica gel column chromatography (eluted with petroleum ether:ethyl acetate=10:1) to give S13-9a.

### Step 10

To a mixture of S13-9a (6.05 g, 27.5 mmol) and 2,6-lutidine (7.36 g, 68.8 mmol) in dry dichloromethane (200 mL) was added drop-wise Trifluoromethanesulfonic anhydride (11.7 g, 41.8 mmol) at −60° C. The reaction mixture was stirred overnight at room temperature, and then quenched with water. The organic layer was separated, washed with diluted 1 N HCl and sat. aq NaHCO₃. The organic layer was separated, dried over anhydrous Na₂SO₄, filtered and concentrated to give a residue, which was purified on silica gel column chromatography (eluted with petroleum ether:ethyl acetate=10:1) to give S13-10a. ¹H NMR (300 MHz, CDCl₃) δ 1.24-1.30 (m, 4H), 2.49-2.55 (m, 1H), 3.08-3.15 (m, 2H), 3.31-3.39 (m, 1H), 4.10-4.23 (m, 2H), 7.06-7.22 (m, 3H).

### Step 11

A mixture of S13-10a (1.25 g, 3.6 mmol), 1,3-bis(diphenylphosphino)propane (147 mg, 0.35 mmol), and Pd(OAc)₂ (40 mg, 0.2 mmol) in dry DMF (20 mL) was heated to 60° C. and then triethylamine (1.08 g, 10.7 mmol), 1-(vinyloxy)butane (3.57 g, 36 mmol) were added and the mixture was stirred at 60° C. overnight. The mixture was cooled to room temperature and acidized with 5% dilute hydrochloric acid, and stirred for 1 hr. The above mixture was poured into water and extracted by ethyl acetate. The organic phase was separated, dried over anhydrous Na₂SO₄, filtered, and concentrated to give a residue, which was purified on silica gel column chromatography (eluted with petroleum ether:ethyl acetate=20:1) to give S13-11a.

### Step 12

To a solution of (S)-diphenyl prolinol (0.18 g, 0.7 mmol) in dry THF (15 mL) was added trimethy borate (0.3 g, 2.88 mmol) at room temperature under N₂ atmosphere. The mixture was stirred at room temperature for 1 hr and then cooled to 0° C. Borane dimethyl sulfide complex (2.0 M in THF, 2.2 mL, 4.4 mmol) was added drop-wise into the above mixture, and the mixture was stirred for 1 hr at 0° C. A solution of S13-11a (0.89 g, 3.6 mmol) in dry THF (10 mL) was added drop-wise into the above mixture at 0° C. in 1 hr. After being allowed to stir for another 30 mins, the reaction was quenched with methanol (10 mL) and concentrated to provide a residue, which was purified by column chromatography (eluted with petroleum ether:ethyl acetate=2:1) to give S13-12a. LC-MS: 269 [M+Na]⁺.

### Step 13

To a mixture of S13-12a (0.9 g, 3.7 mmol) and (R)-oxiran-2-ylmethyl 3-nitrobenzenesulfonate (1.14 g, 4.4 mmol) in dry DMF (10 mL) was added in portion sodium hydrogen (0.18 g, 4.4 mmol) at 0° C. After being allowed to stir for 48 hrs at room temperature, the reaction mixture was poured into water, and extracted with dichloromethane. The combined organic layer was dried over anhydrous Na₂SO₄, filtered, and concentrated to give a residue, which was purified on silica gel column chromatography (eluted with petroleum ether:ethyl acetate=20:1) to give S13-13a. LC-MS: 325 [M+Na]⁺.

### Step 14

The mixture of S13-13a (260 mg, 0.86 mmol) and amines (1.72 mmol) in dry DMF (5 mL) was stirred for 48 hrs at 85° C. The mixture was then poured into water, extracted with dichloromethane and the combined organic layer was dried over anhydrous Na₂SO₄, filtered, and concentrated to give a residue, which was purified on silica gel column chromatography purified by Prep-HPLC to give an intermediate ester S13-14a.

### Step 15

The intermediate ester S13-14a was dissolved in methanol (5 mL) and THF (5 mL) and 2 N sodium hydroxide solution (0.6 mL) was added. After being allowed to stir for 30 mins at 60° C., the reaction mixture was concentrated to remove the organic solvent. The aqueous phase was acidized to pH 3-4 with 2 N dilute hydrochloric acid. The mixture was filtered and the cake was washed with water and dried to give the corresponding targets S13-15a.

The following compounds in Table 13 were made according to EXAMPLE 13.

TABLE 13

| | | FLIPR | |
|---|---|---|---|
| Ex- | | ASSAY | |
| am- | | IC₅₀ | |
| ple | Structure | (nM) | Analysis Data |
| 13-1 | 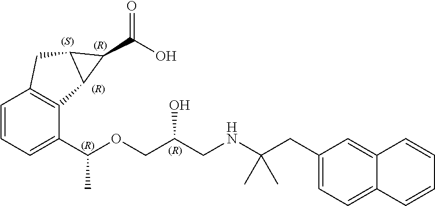 | 28 | ¹HNMR (300 MHz, DMSO-d₆) δ 7.90-7.84 (m, 3H), 2.74-7.72 (m, 1H), 7.50-7.47 (m, 2H), 7.36-7.33 (m, 1H), 7.15-7.05 (m, 3H), 4.64-4.56 (m, 1H), 3.98-3.89 (m, 1H), 3.40-2.67 (m, 1H), 2.36-2.31 (m, 1H), 1.44- 1.42 (m, 3H), 1.23-1.20 (m, 6H), 1.39-1.02 (m, 1H); LC-MS: 474.3 [M + 1]; HPLC: 96.4% |
| 13-2 | 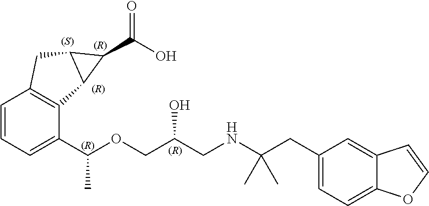 | 47 | ¹H NMR (300 MHz, CD₃OD) δ 7.76-7.75 (m, 1H), 7.52-7.46 (m, 2H), 7.21-7.06 (m, 4H), 6.83-6.82 (m, 1H), 4.68-4.61 (m, 1H), 4.04-3.96 (m, 1H), 3.49-3.44 (m, 1H), 3.33-3.17 (m, 3H), 3.09-2.99 (m, 4H), 2.48- 2.42 (m, 1H), 1.49-1.41 (m, 3H), 1.34 -1.28 (m, 6H), 1.09-1.04 (m, 1H); LC-MS: 464.2 [M + 1]; HPLC 97.6% |
| 13-3 | 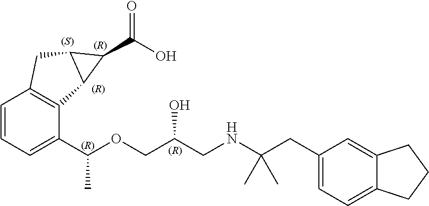 | 88 | ¹H NMR (300 MHz, CD₃OD) δ 7.08-6. 98 (m, 5H), 6.89-6.87 (m, 1H), 4.57-4.51 (m, 1H), 3.91-3.84 (m, 1H), 3.37-3.32 (m, 1H), 3.17-3.07 (m, 3H), 2.97- 2.74 (m, 9H), 2.37-2.31 (m, 1H), 2.01-1.83 (m, 2H), 1.39-1.37 (m, 3H), 1.20-1.18 (m, 6H), 0.98-0.96 (m, 1H); LC-MS: 464.3 [M + 1]; HPLC: 99.9% |
| 13-4 | 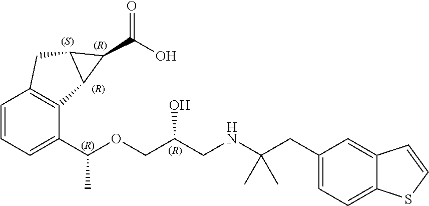 | 36 | ¹H NMR (300 MHz, DMSO-d₆) δ 7.94-7.91 (m, 1H); 7.76-7.72 (m, 2H), 7.44-7.42 (m, 1H), 7.23- 7.09 (m, 4H), 4.66-4.59 (m, 1H), 3.87-3.82 (m, 1H), 3.37-3.20 (m, 3H), 3.09-2.94 (m, 6H), 2.77-2.68 (m, 1H), 2.38-2.33 (m, 1H), 1.46 -1.02 (m, 10H), LC-MS: 480.2 [M + 1]; HPLC: 98.2% |
| 13-5 | 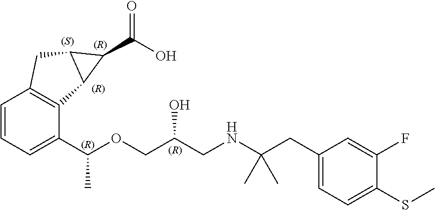 | 130 | ¹H NMR (300 MHz, DMSO-d₆) δ 7.28-7.22 (m, 1H) 7.13-7.01 (m, 5H), 4.63-4.56 (m, 1H), 3.68 (s, 1H), 3.31-2.98 (m, 3H), 2.74-2.65 (m, 3H), 2.47 (s, 3H), 2.34-2.33 (m, 1H), 1.45-1.20 (m, 3H), 1.01-0.97 (m, 6H); LC-MS: 488.2 [M + 1]; HPLC: 96.1% |
| 13-6 | 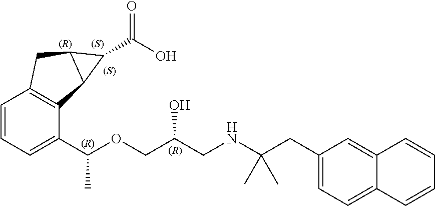 | 4899 | ¹H NMR (301 MHz, CDCl₃) δ 7.87-7.63 (m, 4H), 7.46 (dd, J = 6.1, 3.1 Hz, 2H), 7.32 (d, J = 8.1 Hz, 1H), 7.10-6.93 (m, 2H), 6.92-6.82 (m, 1H), 4.77- 4.42 (m, 1H), 4.42-4.00 (m, 1H), 3.54-2.82 (m, 9H), 2.37-1.93 (m, 2H), 1.51-1.20 (m, 9H). LC- MS: 474.3 [M + 1]. |

### Example 14

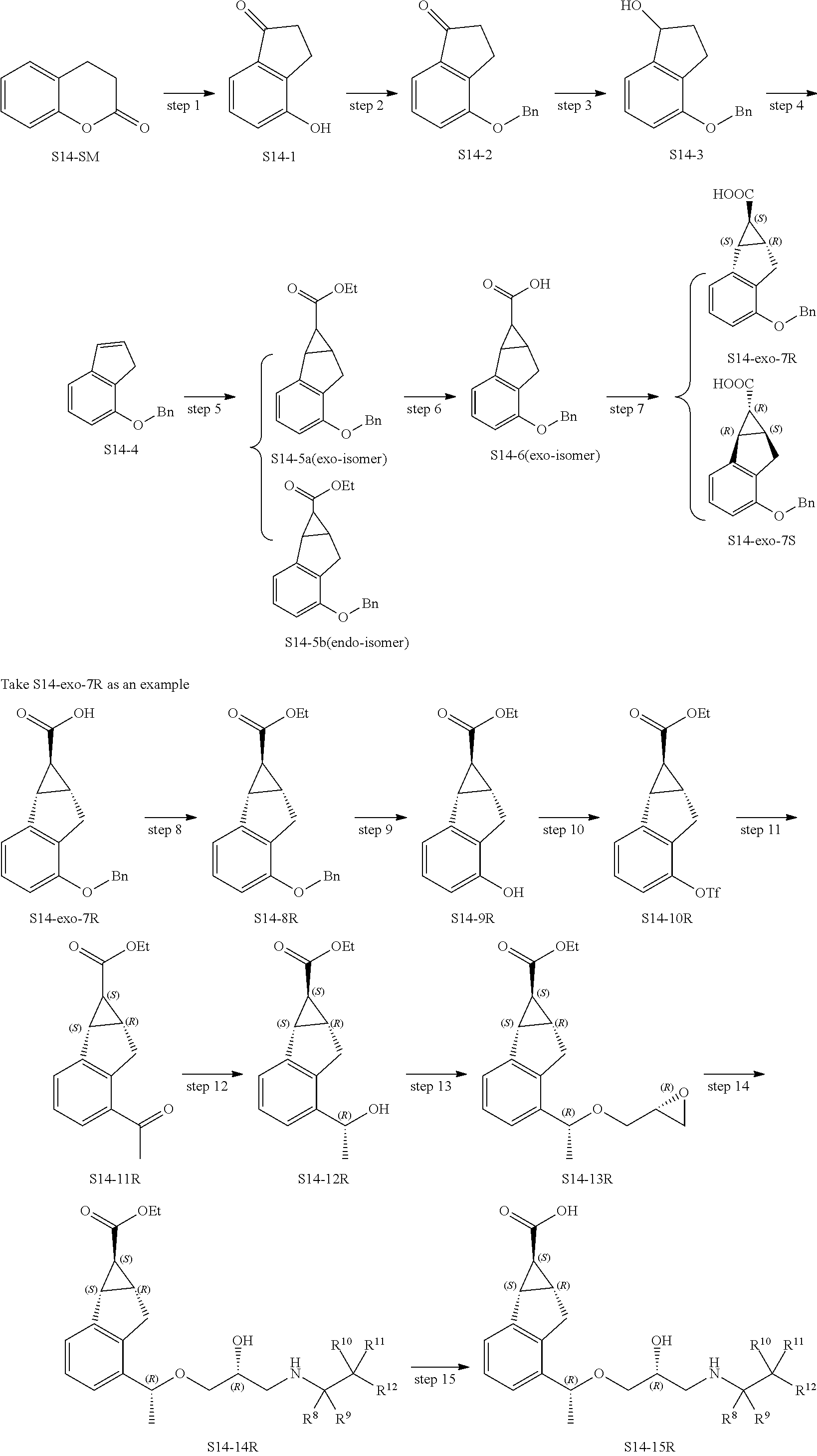

### Experimental Procedure

### Step 1

To the molten salt of AlCl₃ (500 g, 3.75 mol) and NaCl (100 g, 1.71 mol) was added chroman-2-one (S14-SM, 100 g, 0.68 mol) dropwise at 140° C. After being allowed to stir for 30 mins at 180° C.-200° C., the reaction mixture was poured into ice-cooled diluted HCl (2.5 L, 0.5N), and precipitated. The suspension was filtered, and the filter was washed again with diluted aq. HCl (1 L, 0.5N). The combined precipitate was obtained as S14-1.

### Step 2

To a solution of S14-1 (110 g, 0.67 mol) in acetone (1 L) were added K₂CO₃ (186 g, 1.35 mol) and benzyl bromide (81 mL, 0.81 mol). The reaction mixture was stirred at room temperature overnight. The reaction mixture was filtered and the filtrate was concentrated. The residue was dissolved in ethyl acetate and washed with water for two times. Then the organic layer was separated, dried over anhydrous sodium sulfate, filtered and concentrated to obtain a residue, which was purified by chromatography and eluted with dichloromethane: petroleum (1:1) to afford S14-2. ¹H NMR (300 MHz, CDCl₃) δ 7.57-7.21 (m, 7H), 7.07 (m, 1H), 5.16 (s, 2H), 3.10 (dd, J=6.7, 4.8 Hz, 2H), 2.74-2.57 (m, 2H).

### Step 3

To a solution of S14-2 (94.5 g, 0.40 mol) in EtOH (300 mL) was added NaBH₄ (15 g, 0.39 mol). After the reaction was complete, the reaction mixture was quenched with diluted HCl and extracted with ethyl acetate. The organic layer was separated, dried over anhydrous sodium sulfate, filtered and concentrated to afford S14-3 without further purification. ¹H NMR (300 MHz, CDCl₃) δ 7.48-7.29 (m, 5H), 7.21 (t, J=7.8 Hz, 1H), 7.05 (d, J=7.5 Hz, 1H), 6.82 (d, J=8.0 Hz, 1H), 5.31-5.23 (m, 1H), 5.11 (s, 2H), 3.10 (m, 1H), 2.94-2.68 (m, 2H), 2.52 (m, 1H).

### Step 4

To a solution of S14-3 (64 g, 0.27 mol) in toluene (600 mL) was added p-toluenesulfonic acid (1.97 g, 0.03 mol). The mixture was stirred at 80° C. for 1 hr, then it was poured into aq. NaHCO₃. The organic layer was washed with brine, separated, dried over anhydrous sodium sulfate, filtered and concentrated to provide a residue, which was purified by column chromatography to afford S14-4. ¹H NMR (300 MHz, CDCl₃) δ 7.62-7.37 (m, 5H), 7.37-7.28 (m, 1H), 7.17 (d, J=7.4 Hz, 1H), 6.99-6.93 (m, 1H), 6.89 (d, J=8.0 Hz, 1H), 6.66 (m, 1H), 5.26 (s, 2H), 3.55 (s, 2H).

### Step 5

The solution of N₂CHCOOEt (20 mL, 0.16 mol) in 1,2-dichloroethane (30 mL) was dropwise added to a solution of S14-4 (24.2 g, 0.11 mol) and CuBr (114 mg, 0.74 mmol) in 1,2-dichloroethane (120 mL) at 80° C. The reaction mixture was stirred for another 30 mins. Then the mixture was concentrated and the residue was purified by chromatography and eluted with petroleum:ethyl acetate (120:1) to afforded S14-5a (exo-isomers) and S14-5b (endo-isomers).

### Step 6

Aq. NaOH (2N, 34 mL) was added to the solution of S14-5a in THF/CH₃OH (140 mL, 1:1) and the mixture was stirred at 60° C. for 3 hrs. Then the solution was cooled and concentrated. The residue was dissolved in water (160 mL) and extracted with ethyl acetate (50 mL). The aqueous phase was acidified with diluted HCl to PH=2 at 0° C. and the suspension was filtered. S14-6 was obtained.

### Step 7

S14-6 was chirally separated by (R)-1,2,3,4-tetrahydronaphthalen-1-amine and (S)-1,2,3,4-tetrahydronaphthalen-1-amine to get S14-exo-7R and S14-exo-7S. To a solution of S14-6 (11 g, 38.9 mmol) in ethyl acetate (660 mL) was added slowly (R)-1,2,3,4-tetrahydronaphthalen-1-amine and (S)-1,2,3,4-tetrahydronaphthalen-1-amine (3.87 mL, 27.2 mmol) respectively, then the reaction mixture was stirred at room temperature overnight. The suspension was filtered and the solid was washed with ethyl acetate (300 mL) 2 times. The solid (6.18 g) was acidified with diluted HCl (100 mL, 1N) and extracted with ethyl acetate 2 times. The organic layer was separated, dried over sodium sulfate, filtered, and concentrated. S14-exo-7R and S14-exo-7S were obtained.

### Step 8

To a solution of S14-exo-7R (4.5 g, 17 mmol) in DMF were added K₂CO₃ (2.25 g, 17.8 mmol) and C₂H₅I (1.37 mL, 18.7 mmol). Then the mixture was stirred at room temperature for 3.5 hrs. The reaction mixture was poured into water and extracted with ethyl acetate 2 times. The combined organic layer was washed with water and brine. The organic layer was separated, dried over anhydrous sodium sulfate, filtered, and concentrated to afford S14-8R. S14-8S was obtained in the same way.

### Step 9

To a solution of S14-8R (4.9 g, 16 mmol) in EtOH (50 mL) was added Pd/C (0.5 g). The mixture was stirred under H₂ overnight. The reaction mixture was filtered and the filtrate was concentrated. The residue was purified by column chromatography and eluted with petroleum: ethyl acetate (5:1) to afford S14-9R.

### Step 10

The solution of S14-9R (3.2 g, 14.7 mmol) and 2.6-lutidine (4.25 mL, 36.7 mmol) in dry DCM (40 mL) was cooled at −78° C. under N₂. Then Triflic anhydride (3.6 mL, 22 mmol) was slowly added to it. Then the reaction mixture was stirred overnight, allowing to warm up to room temperature. The reaction mixture was quenched with water and washed with diluted HCl (1N, 100 mL). The organic layer was washed with saturated aq. NaHCO₃, separated, dried over anhydrous sodium sulfate, filtered, and concentrated. The residue was purified by chromatography and eluted with petroleum:ethyl acetate (20:1) to afford S14-10R. ¹H NMR (300 MHz, CDCl₃) δ 7.35 (d, J=7.4 Hz, 1H), 7.18 (m, 1H), 7.03 (m, 1H), 4.28-4.02 (m, 2H), 3.76-3.53 (m, 1H), 3.43-3.14 (m, 1H), 3.08-2.88 (m, 1H), 2.54-2.45 (m, 1H), 1.38-1.16 (m, 4H).

### Step 11

Triethylamine (3.8 mL, 0.027 mol) and 1-(vinyloxy)butane (5.25 mL, 0.041 mol) were added to the suspension of S14-10R (3.2 g, 0.009 mol), 1,3-bis(diphenylphosphino)propane (0.38 g, 0.001 mol) and palladium acetate (0.22 g, 0.001 mol) in ethylene glycol (30 mL). Then the mixture was stirred at 60° C. overnight. The reaction mixture was cooled and acidified with diluted HCl (0.7 N, 40 mL). The organic phase was separated, dried over anhydrous sodium sulfate, filtered, and concentrated. The residue was purified by chromatography and eluted with petroleum:ethyl acetate (20:1) to give S14-11R. ¹H NMR (300 MHz, CDCl₃) δ 7.77-7.65 (m, 1H), 7.27 (m, 2H), 4.32-3.84 (m, 2H), 3.65-3.21 (m, 2H), 2.96 (m, 2H), 2.56 (s, 3H), 1.31-1.20 (m, 3H), 1.19-1.07 (m, 1H).

### Step 12

The solution of (S)-diphenyl(pyrrolidin-2-yl) methanol (700 mg, 2.73 mmol) and trimethyl borate (1.27 mL, 11 mmol) in THF (20 mL) was stirred at room temperature for 1 hr. A solution of borane dimethyl sulfide complex solution (2.0 M in THF, 8.27 mL, 27.6 mmol) in THF was slowly added at 0° C. After 45 mins, a solution of S14-11R (3.3 g, 13.8 mmol) in THF (50 mL) was slowly added at 0° C.-10° C. by pump syringe over 3 hrs. After the reaction was complete, the mixture was quenched with MeOH, concentrated, dissolved into ethyl acetate and washed with brine. The organic layer was separated, dried over sodium sulfate, filtered, and concentrated. The residue was purified by chromatography and eluted with petroleum:ethyl acetate (5:1) to afford S14-12R.

### Step 13

To the solution of S14-12R (2.9 g, 11.8 mmol) and (R)-oxiran-2-ylmethyl 3-nitrobenzenesulfonate (3.4 g, 13.0 mol) in DMF (20 mL) was added NaH (0.45 g, 17.7 mmol) at 0° C. The reaction was stirred at room temperature overnight. The mixture was diluted with ethyl acetate and washed with brine. The organic layer was separated, dried over anhydrous sodium sulfate, filtered and concentrated to provide a residue, which was purified by chromatography and eluted with petroleum:ethyl acetate (5:1) to afford S14-13R. ¹H NMR (300 MHz, CDCl₃) δ 7.24 (q, J=4.1 Hz, 1H), 7.18-7.02 (m, 2H), 4.61-4.32 (m, 1H), 4.13 (m, 2H), 3.58-3.42 (m, 1H), 3.37-3.17 (m, 1H), 3.17-3.03 (m, 3H), 2.99-2.80 (m, 1H), 2.78-2.53 (m, 1H), 2.51-2.41 (m, 2H), 1.40 (ddd, J=10.1, 6.2, 4.0 Hz, 3H), 1.25 (ddd, J=7.1, 4.3, 1.8 Hz, 3H), 1.21-1.13 (m, 1H).

### Step 14

To the solution of S14-13R (485 mg, 1.61 mmol) in DMF (4 mL) was added amine (3.22 mmol) at room temperature. The solution was stirred at 85° C. overnight. Then the reaction solution was diluted with ethyl acetate and washed with brine. The organic layer was separated, dried over anhydrous sodium sulfate, filtered and concentrated to provide a residue, which was purified by prep-TLC to afford S14-14R.

### Step 15

2N aq. NaOH (2.5 mL) was added to the solution of S14-14R (0.07 mmol) in MeOH (2.0 mL) and THF (2.0 mL). The solution was stirred at 60° C. for 45 mins. Then MeOH and THF were removed under reduced pressure. 1N aq. HCl was added to the mixture in water (10 mL) to make PH=7. The precipitate was collected and purified by prep-HPLC to afford the product S14-15R.

The following compounds in Table 14 were made according to EXAMPLE 14.

TABLE 14

| | | FLIPR | |
|---|---|---|---|
| Ex- | | ASSAY | |
| am- | | IC₅₀ | |
| ple | Structure | (nM) | Analysis Data |
| 14-1 | 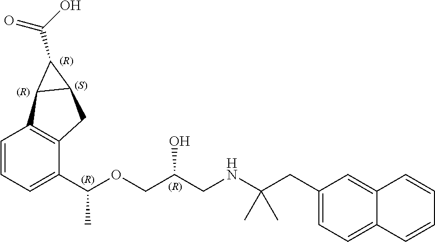 | 270 | ¹H NMR (300 MHz, CD₃OD) δ 7.88 (m, 3H), 7.83 (s, 1H), 7.57-6.90 (m, 6H), 4.50 (m, 1H), 4.02 (m, 1H), 3.40 (m, 2H), 3.31-3.02 (m, 6H), 2.88 (s, 1H), 2.39 (s, 1H), 1.52-1.19 (m, 9H), 1.04 (s, 1H); LC-MS (ESI): 474.3 [M + 1]; HPLC (220 nm): 96.7% |
| 14-2 | 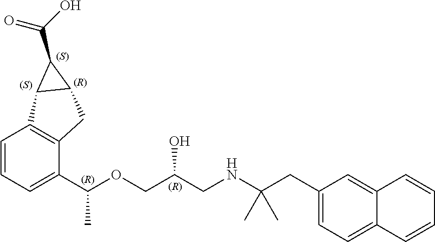 | 150 | ¹H NMR (300 MHz, CD₃OD) δ 7.88 (m, 2H), 7.83 (s, 1H), 7.57-6.90 (m, 6H), 4.50 (m, 1H), 4.02 (m, 1H), 3.40 (m, 2H), 3.31-3.02 (m, 6H), 2.88 (s, 1H), 2.39 (s, 1H), 1.52-1.19 (m, 9H), 1.04 (s, 1H); LC-MS (ESI): 474.3 [M + 1]; HPLC (220 nm): 91% |

### Example 15

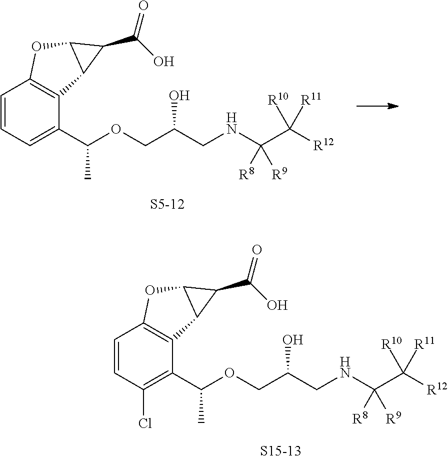

### Experimental Procedure

### Step 1

To a mixture of S5-12 and N-Chlorosuccinimide (5.3 mmol) in THF (10 mL) was added H₂SO₄ (20 μL) at room temperature. The reaction was stirred for 2 days. The mixture was diluted with ethyl acetate and washed with brine. The organic layer was separated, dried over anhydrous sodium sulfate, filtered and concentrated to provide a residue, which was purified by prep-HPLC to afford S15-13.

The following compound in Table 15 was made according to EXAMPLE 15.

TABLE 15

| | | FLIPR | |
|---|---|---|---|
| Ex- | | ASSAY | |
| am- | | IC₅₀ | |
| ple | Structure | (nM) | Analysis Data |
| 15-1 | 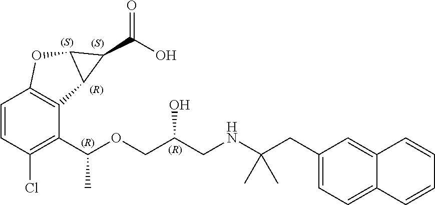 | 17 | ¹H NMR (300 MHz, CD₃OD) δ 7.74 (m, 3H), 7.66 (s, 1H), 7.44-7.32 (m, 2H), 7.28 (dd, J = 8.5, 1.6 Hz, 1H), 7.09 (d, J = 8.6 Hz, 1H), 6.72 (d, J = 8.6 Hz, 1H), 5.12-4.99 (m, 2H), 4.42 (q, J = 7.2 Hz, 1H), 3.99-3.88 (m, 1H), 3.68 (dd, J = 5.3, 3.1 Hz, 1H), 3.43 (dd, J = 9.7, 4.9 Hz, 1H), 3.28 (dd, J = 9.8, 4.9 Hz, 1H), 3.05-3.00 (m, 3H), 1.42 (t, J = 9.6 Hz, 3H), 1.26 (s, 6H), 1.08 (d, J = 2.3 Hz, 1H). LC-MS (ESI) 510.2 [M + 1]. HPLC (220 nm): 97.48%. |

### Example 16

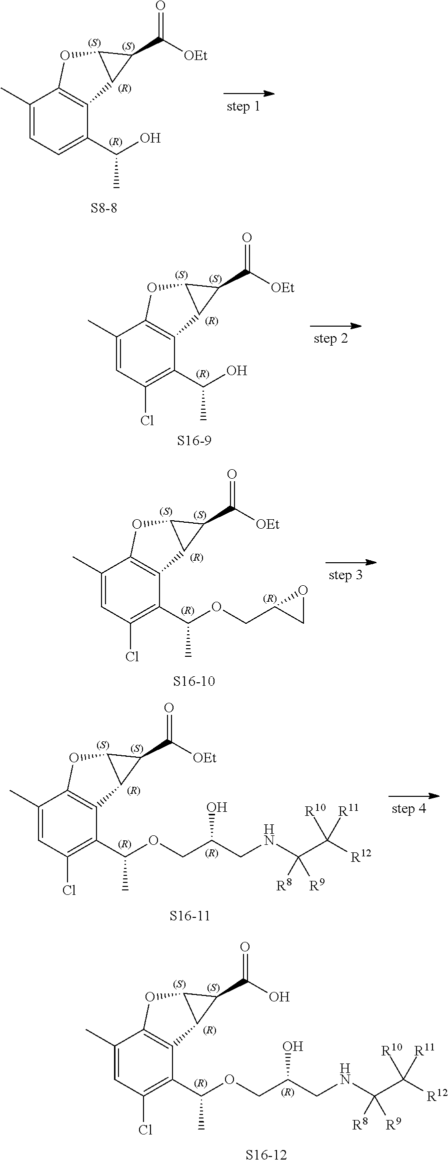

### Experimental Procedure

### Step 1

To a mixture of S8-8 (0.7 g, 2.65 mmol) and N-Chlorosuccinimide (0.96 g, 5.3 mmol) in THF (10 mL) was added H₂SO₄ (20 μL) at room temperature. The reaction was stirred for 2 days. The mixture was diluted with ethyl acetate and washed with brine. The organic layer was separated, dried over anhydrous sodium sulfate, filtered and concentrated to provide a residue, which was purified by column chromatography to afford S16-9. ¹H NMR (CDCl3 300 MHz) δ 6.93-6.74 (m, 2H), 5.10-5.03 (m, 2H), 4.191-4.11 (m, 2H), 3.46-3.35 (m, 1H), 2.18 (s, 3H), 1.88 (s, 1H), 1.53-1.50 (s, 3H), 1.28-1.24 (t, J=6.9 Hz, 3H), 1.21-1.20 (d, J=3 Hz, 1H).

### Step 2

To a mixture of S16-9 (0.7 g, 2.65 mmol) and (R)-oxiran-2-ylmethyl 3-nitrobenzenesulfonate (0.76 g, 2.92 mmol) in DMF (10 mL) was added NaH (0.12 g, 3 mmol) at 0° C. The reaction was stirred at room temperature for 2 days. The mixture was diluted with ethyl acetate and washed with brine. The organic layer was separated, dried over anhydrous sodium sulfate, filtered and concentrated to provide a residue, which was purified by column chromatography to afford S16-10. ¹H NMR (CDCl3 300 MHz) δ 6.93-6.74 (m, 2H), 5.06-5.04 (d, J=5.4 Hz, 2H), 4.67-4.61 (m, 1H), 4.18-4.11 (q, J=6.9 Hz, 2H), 3.63-3.60 (m, 1H), 3.43-3.41 (m, 1H), 3.20-3.10 (m, 2H), 2.74-2.71 (m, 1H), 2.48-2.47 (m, 1H), 2.17 (s, 3H), 1.49-1.46 (m, 3H), 1.27-1.22 (m, 3H), 1.16-1.13 (m, 1H).

### Step 3

To a solution of S16-10 (100 mg, 0.3 mmol) in DMF (2 mL) was added amine (126 mg, 0.629 mmol) at room temperature. The solution was stirred overnight. Then the reaction solution was extracted with ethyl acetate and washed with brine. The organic layer was separated, dried over anhydrous sodium sulfate, filtered and concentrated to provide a residue, which was purified by prep-TLC to afford S16-11.

### Step 4

2N aq. NaOH (0.5 mL) was added to S16-11 (0.07 mmol) in MeOH (1.0 mL) and THF (1.0 mL). The solution was stirred at 60° C. for 45 m. Then MeOH and THF were removed under reduced pressure. 1N aq. HCl was added to the mixture in water (10 mL) to make PH=7. The precipitate was collected and purified by prep-HPLC to afford the product S16-12.

The following compounds in Table 16 were made according to EXAMPLE 16.

TABLE 16

| | | FLIPR | |
|---|---|---|---|
| Ex- | | ASSAY | |
| am- | | IC₅₀ | |
| ple | Structure | (nM) | Analysis Data |
| 16-1 | 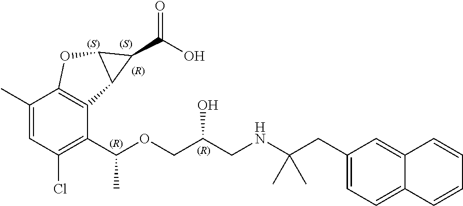 | 9 | ¹H NMR (MeOD, 300 MHz) δ 7.88-7.85 (m, 3H), 7.79-7.76 (s, 1H), 7.50-7.32 (m, 3H), 7.09-7.06 (s, 1H), 5.15-5.13 (m, 2H), 4.03 (m, 1H), 3.76-3.74 (m, 1H), 3.59-3.53 (m, 1H), 3.42-3.38 (m, 1H), 3.20-3.18 (m, 3H), 2.16 (s, 3H), 1.50-1.48 (d, J = 6.0 Hz, 3H), 1.37 (s, 6H), 1.17-1.16 (m, 1H); LC-MS (ESI) 523 [M + 1]; HPLC (220 nm): 100.0%. |
| 16-2 | 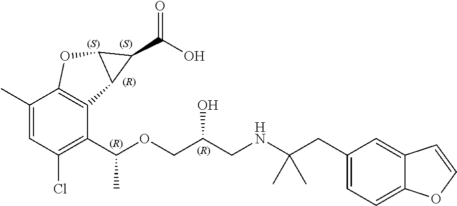 | 17 | ¹H NMR (MeOD, 300 MHz) δ 7.77-7.76 (s, 1H), 7.51-7.48 (m, 2H), 7.24-7.22 (d, 1H, J = 6.0 Hz), 7.04 (s, 1H), 6.83-6.82 (d, 1H, J = 3.0 Hz), 5.15- 5.13 (m, 2H), 4.00 (m, 1H), 3.78-3.76 (m, 1H), 3.58- 3.53 (m, 1H), 3.41-3.37 (m, 1H), 3.21-3.19 (m, 3H), 2.16 (s, 3H), 1.51-1.49 (d, 3H, J = 6.0 Hz), 1.15-1.14 (m, 1H); LC-MS (ESI) 514 [M + 1]; HPLC (220 nm): 95.7%. |
| 16-3 | 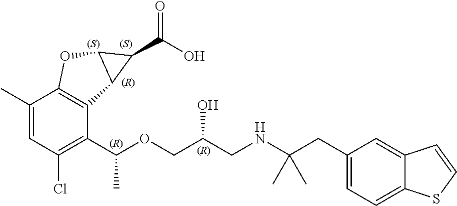 | 14 | ¹H NMR (301 MHz, cd₃od) δ 7.89 (d, J = 8.3 Hz, 1H), 7.75 (s, 1H), 7.60 (d, J = 5.4 Hz, 1H), 7.36 (d, J = 5.5 Hz, 1H), 7.29-7.20 (m, 1H), 7.03 (s, 1H), 5.18-5.06 (m, 2H), 4.02 (dd, J = 9.3, 3.0 Hz, 1H), 3.71 (dd, J = 5.2, 3.0 Hz, 1H), 3.51 (dd, J = 9.8, 4.9 Hz, 1H), 3.43-3.34 (m, 1H), 3.27 (d, J = 2.9 Hz, 1H), 3.16-3.01 (m, 3H), 2.16 (s, 3H), 1.51 (t, J = 8.8 Hz, 3H), 1.34 (s, 6H), 1.13 (s, 1H). LC-MS (ESI) 530.2 [M + 1]; HPLC (220 nm): 97.32%. |
| 16-4 | 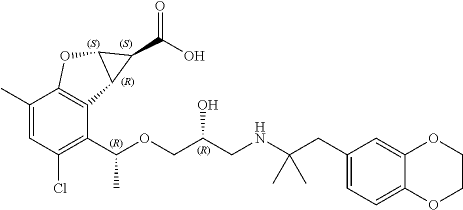 | 41 | ¹H NMR (301 MHz, cd₃od) δ 7.04 (s, 1H), 6.80 (d, J = 8.2 Hz, 1H), 6.76-6.63 (m, 2H), 5.20- 5.07 (m, 2H), 4.22 (s, 4H), 4.00 (d, J = 6.3 Hz, 1H), 3.73 (dd, J = 5.4, 3.0 Hz, 1H), 3.50 (dd, J = 9.9, 4.9 Hz, 1H), 3.36 (d, J = 5.2 Hz, 1H), 3.23 (dd, J = 12.3, 3.0 Hz, 2H), 3.10-2.99 (m, 1H), 2.84 (s, 2H), 2.17 (s, 3H), 1.52 (d, J = 6.5 Hz, 3H), 1.29 (s, 6H), 1.16 (d, J = 2.1 Hz, 1H). LC- MS (ESI) 532.2 [M + 1]; HPLC (220 nm): 99.36%. |
| 16-5 | 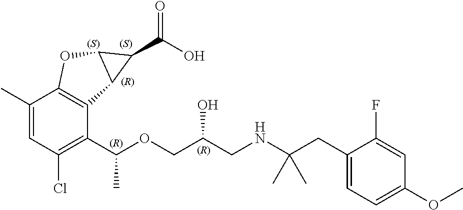 | 24 | ¹H NMR (301 MHz, cd₃od) δ 7.19 (t, J = 8.7 Hz, 1H), 7.05 (s, 1H), 6.79-6.71 (m, 2H), 5.14 (dd, J = 7.3, 5.9 Hz, 2H), 4.02 (d, J = 6.1 Hz, 1H), 3.80 (s, 3H), 3.75 (dd, J = 5.4, 3.1 Hz, 1H), 3.51 (dd, J = 9.8, 4.9 Hz, 1H), 3.41-3.35 (m, 2H), 3.13-3.02 (m, 2H), 2.94 (t, J = 9.2 Hz, 2H), 2.17 (s, 3H), 1.52 (d, J = 6.5 Hz, 3H), 1.30 (s, 6H), 1.16 (d, J = 2.1 Hz, 1H). LC-MS (ESI) 522.2 [M + 1]; HPLC (220 nm): 98.54%. |
| 16-6 | 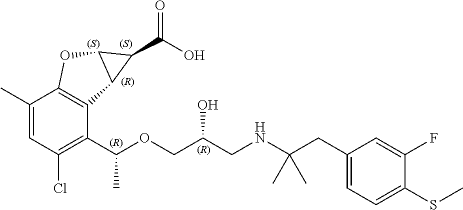 | 17 | ¹H NMR (MeOD, 300 MHz) δ 7.33-7.31 (m, 1H), 7.08-7.01 (m, 3H), 5.17-5.14 (m, 1H), 4.01 (m, 1H), 3.79-3.76 (m, 1H), 3.53-3.48 (m, 1H), 3.38- 3.36 (m, 1H), 3.13-3.04 (m, 1H), 2.95 (s, 2H), 2.46 (s, 3H), 2.16 (s, 3H), 1.54-1.51 (d, 3H, J = 9.0 Hz), 1.38 (s, 6H), 1.16-1.15 (m, 1H); LC-MS (ESI) 537 [M + 1]; HPLC (220 nm): 97.0%. |

### Example 17

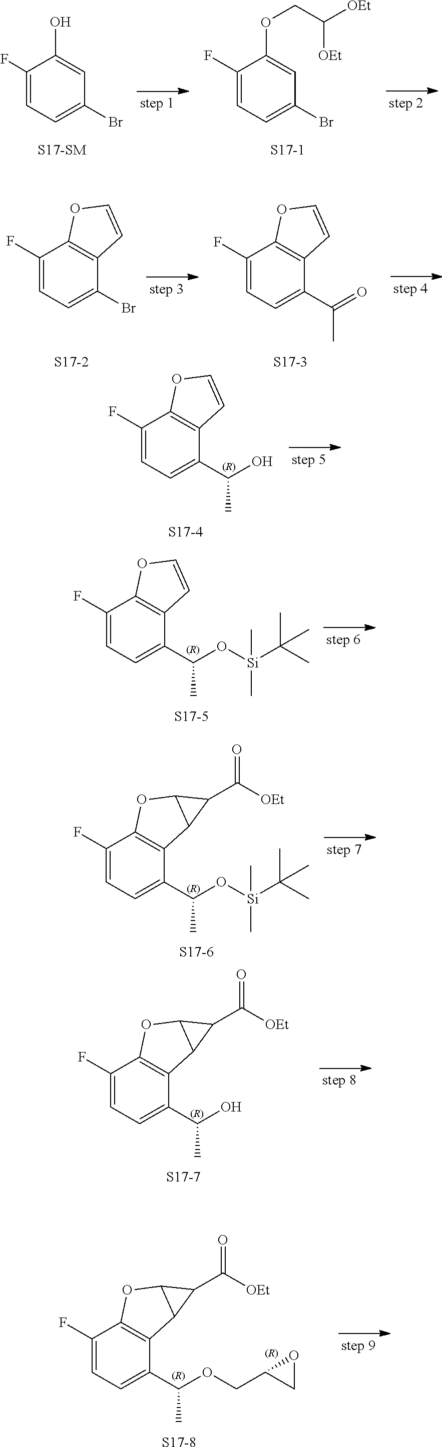

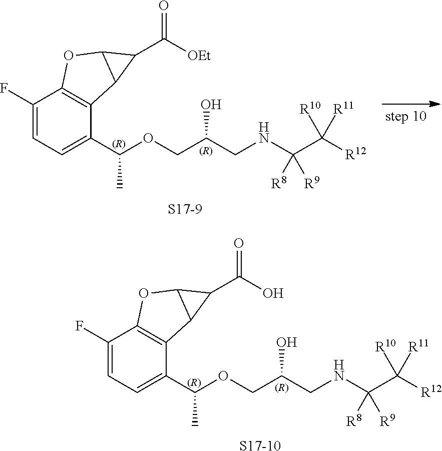

### Experimental Procedure

### Step 1

To a suspension of 5-bromo-2-fluorophenol (S17-SM, 30 g, 158 mmol) and potassium carbonate (32 g, 237 mmol) in DMF (180 mL) was added 2-bromo-1,1-diethoxyethane (26 mL, 173 mmol) at 135° C. The reaction mixture was stirred at 135° C. for 7 hrs. After the solvent was removed, the residue was diluted with ethyl acetate and washed with brine. The organic layer was separated, dried over anhydrous sodium sulfate, filtered and concentrated in vacuo to give product which was purified by column chromatography (eluted with petroleum ether:ethyl acetate=50:1) to give product S17-1. ¹H NMR (300 MHz, CDCl₃) δ 7.13 (dd, J=7.5, 2.2 Hz, 1H), 6.97 (ddd, J=19.3, 7.2, 5.5 Hz, 2H), 4.83 (t, J=5.2 Hz, 1H), 4.04 (d, J=5.2 Hz, 2H), 3.86-3.71 (m, 2H), 3.71-3.54 (m, 2H), 1.25 (t, J=7.0 Hz, 6H).

### Step 2

The mixture of S17-1 (38 g, 124 mmol) and polyphosphoric acid (76 g, 222 mmol) in chlorobenzene was stirred at 130° C. for 3 hrs. Then the reaction mixture was cooled to ambient temperature. Chlorobenzene was decanted and the black residue was neutralized with aqueous NaHCO₃, and extracted with ethyl acetate. The combined organic layer was washed with aqueous NaHCO₃. The organic layer was separated, dried over anhydrous sodium sulfate, filtered and concentrated. The obtained residue was purified by column chromatography and eluted with petroleum ether to afford S17-2. ¹H NMR (CDCl₃ 300 MHz) δ 7.74 (d, J=2.2 Hz, 1H), 7.35 (dd, J=8.5, 3.7 Hz, 1H), 6.99 (dd, J=10.3, 8.5 Hz, 1H), 6.89 (dd, J=2.8, 2.2 Hz, 1H).

### Step 3: S17-2

(10 g, 46.7 mmol), 1,3-bis(diphenylphosphino)propane (1.92 g, 4.67 mmol), ethylene glycol (20 mL), and palladium acetate (530 mg, 2.3 mmol) was added into a flask under nitrogen. To the mixture were added triethylamine (20 mL, 140 mmol) and butyl vinyl ether (27 mL, 210 mmol). The resultant mixture was stirred at 125° C. under nitrogen for 6 hrs, cooled to ambient temperature, diluted with water, and extracted with ethyl acetate. The organic layer was separated, dried over anhydrous Na₂SO₄, filtered and concentrated. The residue was purified by column chromatography (eluted with petroleum ether:ethyl acetate=20:1) to give ketal. The ketal was treated with 1N HCl to give target S17-3. ¹H NMR (300 MHz, CDCl₃) δ 7.82-7.76 (m, 2H), 7.60 (dd, J=3.1, 2.1 Hz, 1H), 7.08 (m, 1H), 2.67 (s, 3H).

### Step 4

A mixture of (S)-diphenylprolinol (0.57 g, 2.25 mmol) and trimethyl borate (1 mL, 9.0 mmol) in anhydrous THF (7 mL) was stirred at ambient temperature for 2 hrs. To the mixture was added dropwise borane dimethylsulfane ether (6.7 mL, 13.4 mmol) at 0° C. The reaction mixture was stirred at ambient temperature for 4 hrs. A solution of S17-3 (2.0 g, 11.2 mmol) in THF (13 mL) was added dropwise via syringe pump at a temperature between −40° C. and −20° C. for 3 h. The resulting mixture was stirred overnight. TLC indicated the completion of the reaction. The reaction was quenched with methanol and concentrated in vacuo. The residue was purified by column chromatography (eluted with petroleum ether:ethyl acetate=15:1), to give product S17-4.

### Step 5

To a solution of S17-4 (2.0 g, 11.1 mmol) in DMF (20 mL) was added imidazole (1.5 g, 22.2 mmol), followed by tert-butyldimethylchlorosilane (TBDMSCl, 2.5 g, 16.7 mmol) at 0° C. The mixture was stirred at ambient temperature overnight. Then the reaction solution was diluted with ethyl acetate and washed with brine. The organic layer was separated, dried over anhydrous Na₂SO₄, filtered and concentrated. The residue was purified by column chromatography (eluted with petroleum ether:ethyl acetate=50:1) to give product S17-5.

### Step 6

To a solution of S17-5 (6.7 g, 22.7 mmol) in 1,2-dichloroethane (30 mL) was added rhodium(II) acetate (705 mg, 1.6 mmol) under nitrogen. The mixture was heated to 85° C. To the mixture was added dropwise a solution of ethyl diazoethanoate (11.7 g, 102 mmol) in 1,2-dichloroethane (40 mL) via syringe pump for 3 hrs. The reaction was stirred at 85° C. for 2 hrs. The mixture was concentrated and diluted with water and extracted with ethyl acetate. The organic layer was separated, dried over anhydrous sodium sulfate, filtered, and concentrated. The residue was purified by column chromatography (eluted with petroleum ether:ethyl acetate=30:1) to give product S17-6. ¹H NMR (CDCl₃ 300 MHz) δ 6.98-6.72 (m, 2H), 5.19-5.08 (m, 1H), 4.94 (m, 1H), 4.24-4.08 (m, 2H), 3.45 (m, 1H), 1.42 (dd, J=8.5, 6.4 Hz, 3H), 1.33-1.22 (m, 4H), 0.86 (t, J=3.8 Hz, 9H), 0.05 (d, J=3.6 Hz, 3H), −0.05 (d, J=13.6 Hz, 3H).

### Step 7

To a solution of S17-6 (200 mg, 0.5 mmol) in THF (2.5 mL) was added dropwise a solution of Tetrabutylammonium fluoride (275 mg, 1.0 mmol) in THF (2.5 mL) at 0° C. The reaction was stirred at ambient temperature overnight and quenched with water and extracted with ethyl acetate. The organic layer was separated, dried over anhydrous sodium sulfate, filtered, and concentrated. The residue was purified by column chromatography (eluted with petroleum ether:ethyl acetate=10:1) to give product S17-7.

### Step 8

To a mixture of S17-7 (2.3 g, 8.6 mmol) and (R)-oxiran-2-ylmethyl 3-nitrobenzenesulfonate (2.46 g, 9.5 mmol) in anhydrous DMF (5 mL) was added NaH (380 mg, 95 mmol) at 0° C. The reaction was stirred at ambient temperature for 2 days. The mixture was diluted with water and extracted with ethyl acetate. The organic layer was washed with brine. The organic layer was separated, dried over anhydrous sodium sulfate, filtered and concentrated. The residue was purified by column chromatography (petroleum ether:ethyl acetate=7:1) to give product S17-8.

### Step 9

To a solution of S17-8 (200 mg, 0.62 mmol) in DMF (2 mL) was added amine (0.68 mmol) at ambient temperature. The solution was stirred at 85° C. for 48 hrs. Solvent was removed. The residue was purified by prep-TLC (eluted with DCM:MeOH=20:1) to give product S17-9 as a pale yellow foam.

### Step 10

1 N aq. NaOH (1.5 mL) was added to S17-9 (0.38 mmol) in MeOH (1.5 mL) and THF (2.0 mL) at 0° C. The solution was stirred at 60° C. for 2 hrs. Then solvent was removed under reduced pressure. The residue was dissolved in water. 1 N aq. HCl was added to the solution to acidify pH=2. The precipitate was collected and dissolved in dichloromethane. The solution was dried over anhydrous sodium sulfate, filtered, and concentrated to give product which was purified by prep-HPLC to afford S17-10.

The following compounds in Table 17 were made according to EXAMPLE 17.

TABLE 17

| | | FLIPR | |
|---|---|---|---|
| Ex- | | ASSAY | |
| am- | | IC₅₀ | |
| ple | Structure | (nM) | Analysis Data |
| 17-1 | 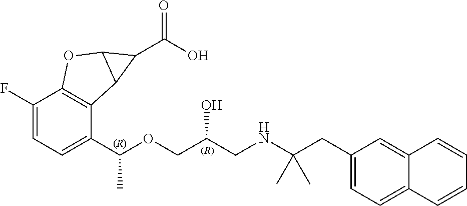 | 26 | ¹H NMR (300 MHz, CD₃OD) δ 7.85 (d, J = 8.0 Hz, 3H), 7.77 (s, 1H), 7.48 (dd, J = 6.5, 2.6 Hz, 2H), 7.40 (d, J = 8.4 Hz, 1H), 7.04 -6.91 (m, 1H), 6.91-6.79 (m, 1H), 5.23 (d, J = 5.3 Hz, 1H), 4.72- 4.55 (m, 1H), 4.04 (s, 1H), 3.64-3.37 (m, 3H), 3.21-3.01 (m, 3H), 1.49-1.34 (m, 9H), 1.24 (m, 1H); LC-MS 494 [M + 1]: HPI.C (220 nm): 97.1%. |
| 17-2 | 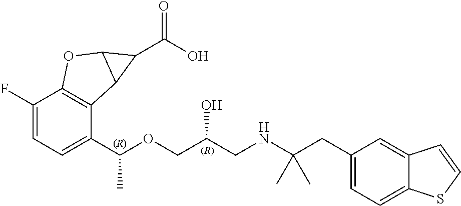 | N/A | ¹H NMR (300 MHz, CD₃OD) δ 7.88 (d, J = 8.2 Hz, 1H), 7.77 (s, 1H), 7.59 (dd, J = 5.4, 1.4 Hz, 1H), 7.36 (d, J = 5.4 Hz, 1H), 7.26 (d, J = 8.3 Hz, 1H), 6.97 (dd, J = 10.5, 8.5 Hz, 1H), 6.84 (dd, J = 8.4, 4.2 Hz, 1H), 5.21 (d, J = 5.3 Hz, 1H), 4.64 (dd, J = 16.9, 6.5 Hz, 1H), 4.05 (s, 1H), 3.65-3.36 (m, 3H), 3.20-2.98 (m, 3H), 1.48-1.31 (m, 9H), 1.22 (m, 1H); LC-MS 500 [M + 1]; HPLC (220 nm): 98.6%. |
| 17-3 | 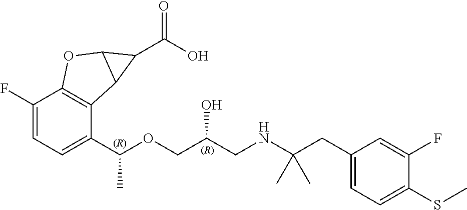 | N/A | ¹H NMR (300 MHz, CD₃OD) δ 7.29 (t, J = 8.0 Hz, 1H), 7.12-6.90 (m, 3H), 6.90-6.80 (m, 1H), 5.20 (dd, J = 4.3, 3.1 Hz, 1H), 4.66 (dq, J = 12.9, 6.3 Hz, 1H), 4.03 (s, 1H), 3.46 (m, 3H), 3.11-2.93 (m, 3H), 2.46 (d, J = 1.4 Hz, 3H), 1.50 (dd, J = 12.9, 6.5 Hz, 3H), 1.31 (s, 6H), 1.25-1.16 (m, 1H); LC-MS 508 [M+ 1]; HPLC (220 nm): 98.1%. |

### Example 18

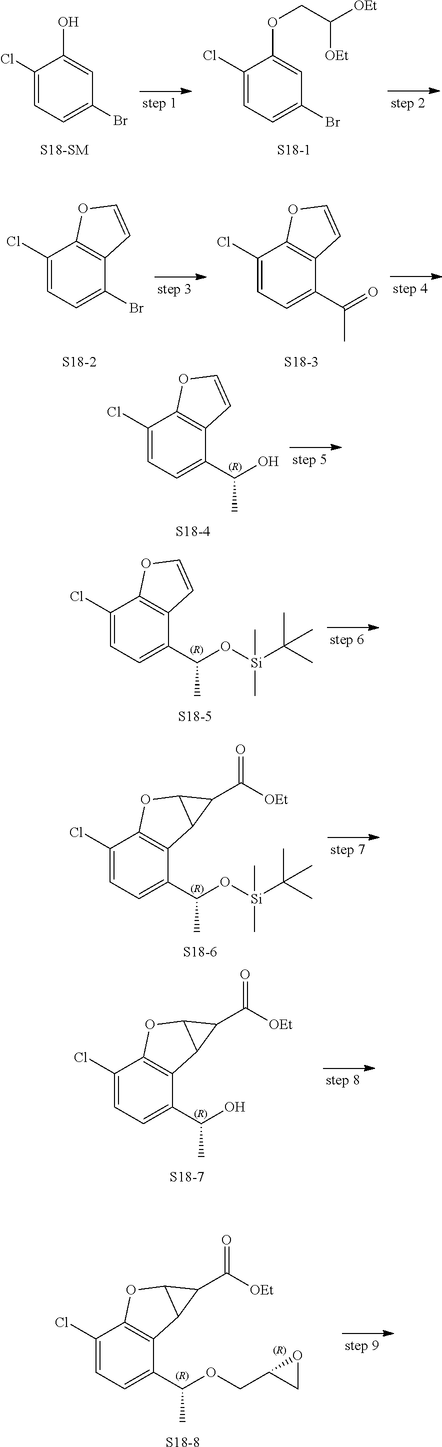

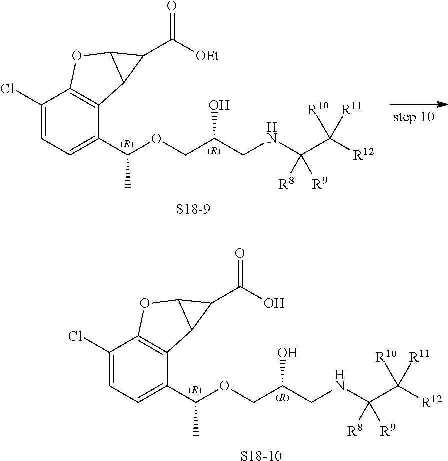

### Experimental Procedure

### Step 1

To a suspension of 5-bromo-2-chlorophenol (S18-SM, 10 g, 48 mmol) and potassium carbonate (10.9 g, 79 mmol) in DMF was added 2-bromo-1,1-diethoxyethane (8.9 mL, 58 mmol) at 135° C. The reaction mixture was stirred at 135° C. for 7 hrs. After the solvent was removed, the residue was diluted with ethyl acetate and washed with brine. The organic layer was separated, dried over anhydrous Na₂SO₄, filtered and concentrated in vacuo to give product which was purified by column chromatography to give S18-1. ¹H NMR (300 MHz, CDCl3) δ 7.53 (d, J=2.4 Hz, 1H), 7.34 (dd, J=8.8, 2.4 Hz, 1H), 6.85 (d, J=8.8 Hz, 1H), 4.89 (t, J=5.2 Hz, 1H), 4.07 (d, J=5.2 Hz, 2H), 3.71 (m, 7.0 Hz, 4H), 1.34-1.23 (m, 6H).

### Step 2

The mixture of S18-1 (15 g, 46.5 mmol) and polyphosphoric acid (13 g, 38 mmol) in chlorobenzene was stirred at 130° C. for 3 hrs. Then the reaction mixture was cooled to ambient temperature. Chlorobenzene was decanted and the black residue was neutralized with aqueous NaHCO₃, and extracted with ethyl acetate. The combined organic layer was washed with aq. Na₂CO₃. The organic layer was separated, dried over anhydrous Na₂SO₄, filtered and concentrated. The obtained residue was purified by column chromatography and eluted with petroleum ether to afford S18-2. ¹NMR (CDCl3 300 MHz) δ 7.69 (d, J=2.2 Hz, 1H), 7.64 (d, J=1.8 Hz, 1H), 7.45 (d, J=1.5 Hz, 1H), 6.77 (d, J=2.2 Hz, 1H).

### Step 3: S18-2

(2.4 g, 10.4 mmol), 1,3-bis(diphenylphosphino)propane (462 mg, 1.12 mmol), ethylene glycol (8 mL), and palladium acetate (127 mg, 0.56 mmol) were added into a flask under nitrogen. To the mixture were added triethylamine (3.4 g, 33.6 mmol) and butyl vinyl ether (5 g, 50.4 mmol). The resulting mixture was stirred at 125° C. under nitrogen for 6 hrs. It was cooled to ambient temperature, diluted with water, and extracted with ethyl acetate. The organic layer was separated, dried over anhydrous Na₂SO₄, filtered and concentrated. The residue was purified by column chromatography (eluted with petroleum ether:ethyl acetate=20:1) to give ketal. The ketal was treated with 1N HCl to give S18-3. ¹H NMR (300 MHz, CDCl₃) δ 8.14 (d, J=1.6 Hz, 1H), 7.97 (d, J=1.5 Hz, 1H), 7.76 (d, J=2.2 Hz, 1H), 6.92 (d, J=2.2 Hz, 1H), 2.66 (s, 3H).

### Step 4

A mixture of (S)-diphenylprolinol (1.0 g, 3.9 mmol) and trimethyl borate (1.6 g, 15.6 mmol) in anhydrous THF (15 mL) was stirred at ambient temperature for 2 hrs. To the mixture was added dropwise borane dimethylsulfane ether (12 mL, 23.5 mmol) at 0° C. The reaction mixture was stirred at ambient temperature for 4 hrs. A solution of S18-3 (3.8 g, 19.6 mmol) in THF (25 mL) was added dropwise via syringe pump at −40° C. ˜−20° C. for 5 hrs. The resulting mixture was stirred overnight. TLC indicated the completion of the reaction. The reaction was quenched with methanol and concentrated in vacuo. Water was added, and the mixture was extracted with ethyl acetate. The extract was dried over anhydrous NaₛSO₄, filtered and concentrated. The residue was purified by column chromatography (eluted with petroleum ether:ethyl acetate=15:1) to give S18-4.

### Step 5

To a solution of S18-4 (2.0 g, 11.1 mmol) in DMF (20 mL) was added imidazole (1.5 g, 22.2 mmol), followed by tert-butyldimethylchlorosilane (TBDMSCl, 2.5 g, 16.7 mmol) at 0° C. The mixture was stirred at ambient temperature overnight. Then the reaction solution was diluted with ethyl acetate and washed with brine. The organic layer was separated, dried over anhydrous sodium sulfate, filtered and concentrated. The residue was purified by column chromatography to give S18-5.

### Step 6

To a solution of S18-5 (500 mg, 1.6 mmol) in 1,2-dichloroethane (5 mL) was added rhodium(II) acetate (35 mg, 0.08 mmol) under nitrogen. The mixture was heated to 85° C. To the mixture was added dropwise ethyl diazoethanoate (730 mg, 6.4 mmol) in 1,2-dichloroethane (5 mL) via syringe pump for 3 hrs. The reaction was stirred at 85° C. for 2 hrs. The mixture was concentrated, diluted with water, and extracted with ethyl acetate. The organic layer was separated, dried over anhydrous sodium sulfate, filtered, and concentrated. The residue was purified by column chromatography (eluted with petroleum ether:ethyl acetate=30:1) to give product S18-6.

### Step 7

To a solution of S18-6 (400 mg, 1.0 mmol) in THF (5 mL) was added dropwise a solution of Tetrabutylammonium fluoride (550 mg, 2.1 mmol) in THF (5 mL) at 0° C. The reaction was stirred at ambient temperature overnight, quenched with water and extracted with ethyl acetate. The organic layer was separated, dried over anhydrous sodium sulfate, filtered, and concentrated. The residue was purified by column chromatography (eluted with petroleum ether:ethyl acetate=10:1) to give S18-7. ¹H NMR (CDCl₃, 300 MHz) δ 7.30 (dd, J=3.2, 1.7 Hz, 1H), 7.18 (dd, J=3.2, 1.7 Hz, 1H), 5.14 (dt, J=5.4, 1.0 Hz, 1H), 4.82 (q, J=6.4 Hz, 1H), 4.19 (m, 2H), 3.27 (m, 1H), 1.46 (d, J=6.4 Hz, 3H), 1.36-1.28 (m, 3H), 1.22 (s, 1H). LC-MS: 283 [M+1].

### Step 8

To a mixture of S18-7 (500 mg, 1.8 mmol) and (R)-oxiran-2-ylmethyl 3-nitrobenzenesulfonate (535 mg, 2.0 mmol) in anhydrous DMF (5 mL) was added NaH (81 mg, 2.0 mmol) at 0° C. The reaction was stirred at ambient temperature for 3 days. The mixture was diluted with water, and extracted with ethyl acetate. The organic layer was washed with brine. The organic layer was separated, dried over anhydrous sodium sulfate, filtered and concentrated, The residue was purified by column chromatography (eluted with petroleum ether:ethyl acetate=7:1) to give S18-8. ¹H NMR (CDCl₃, 300 MHz) δ 7.21 (s, 1H), 7.08 (s, 1H), 5.12 (m, 1H), 4.40 (m, 1H), 4.13 (m, 2H), 3.55 (m, 1H), 3.25 (s, 1H), 3.10 (m, 2H), 2.72 (m, 1H), 2.46 (s, 1H), 1.40 (d, J=6 Hz, 3H), 1.32 (m, 1H), 1.25 (m, 3H).

### Step 9

To a solution S18-8 (110 mg, 0.32 mmol) in DMF (2 mL) was added amine (0.035 mmol) at ambient temperature. The solution was stirred at 85° C. for 48 hrs. Solvent was removed. The residue was purified by prep-TLC (DCM:MeOH=20:1) to give S18-9.

### Step 10

Aq. NaOH (0.33N, 3 mL) was added to S18-9 (0.26 mmol) in MeOH (3.0 mL) and THF (4.0 mL) at 0° C. The solution was stirred at 60° C. for 2 hrs. Then solvent was removed under reduced pressure. The residue was dissolved in water. 1 N aq. HCl was added to the solution to acidify pH=2. The precipitate was collected and dissolved in dichloromethane. The solution was dried over anhydrous sodium sulfate, filtered, and concentrated to give product which was purified by prep-HPLC to afford S18-10.

The following compound in Table 18 was made according to EXAMPLE 18.

TABLE 18

| | | FLIPR | |
|---|---|---|---|
| Ex- | | ASSAY | |
| am- | | IC₅₀ | |
| ple | Structure | (nM) | Analysis Data |
| 18-1 | 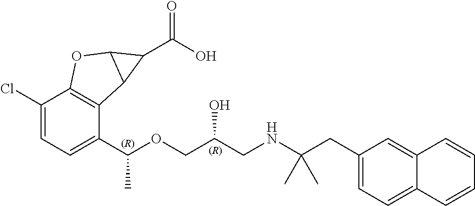 | 40 | ¹H NMR (300 MHz, CD₃OD) δ 7.86 (dd, J = 8.8, 3.2 Hz, 3H), 7.77 (s, 1H), 7.56-7.44 (m, 2H), 7.42-7.27 (m, 2H), 7.17 (s, 1H), 5.21 (d, J = 5.3 Hz, 1H), 4.41 (q, J = 6.3 Hz, 1H), 3.99 (d, J = 4.6 Hz, 1H), 3.46 (dd, J = 9.9, 4.8 Hz, 1H), 3.37-3.33 (m, 2H), 3.29-3.27 (m, 1H), 3.19-3.03 (m, 3H), 1.38 (s, 6H), 1.32 (dd, J = 6.3, 4.0 Hz, 3H), 1.21 (t, J = 2.7 Hz, 1H). LC-MS 510 [M + 1]; HPLC (220 nm): 99.6%. |
| 18-2 | 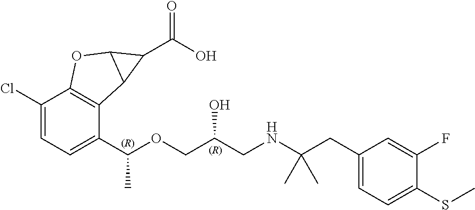 | N/A | ¹H NMR (300 MHz, CD₃OD) δ 7.38 (d, J = 1.6 Hz, 1H), 7.31 (t, J = 8.0 Hz, 1H), 7.20 (d, J = 1.5 Hz, 1H), 7.10-6.99 (m, 2H), 5.25-5.18 (m, 1H), 4.45 (q, J = 6.3 Hz, 1H), 3.97 (d, J = 5.9 Hz, 1H), 3.45 (dd, J = 9.9, 5.0 Hz, 1H), 3.39-3.33 (m, 2H), 3.26 (d, J = 12.4 Hz, 1H), 3.10-2.89 (m, 3H), 2.46 (d, J = 3.0 Hz, 3H), 1.40 (d, J = 6.4 Hz, 3H), 1.31 (s, 6H), 1.23-1.19 (m, 1H). LC-MS 524 [M + 1]; HPLC (220 nm): 99.6%. |

### Example 19

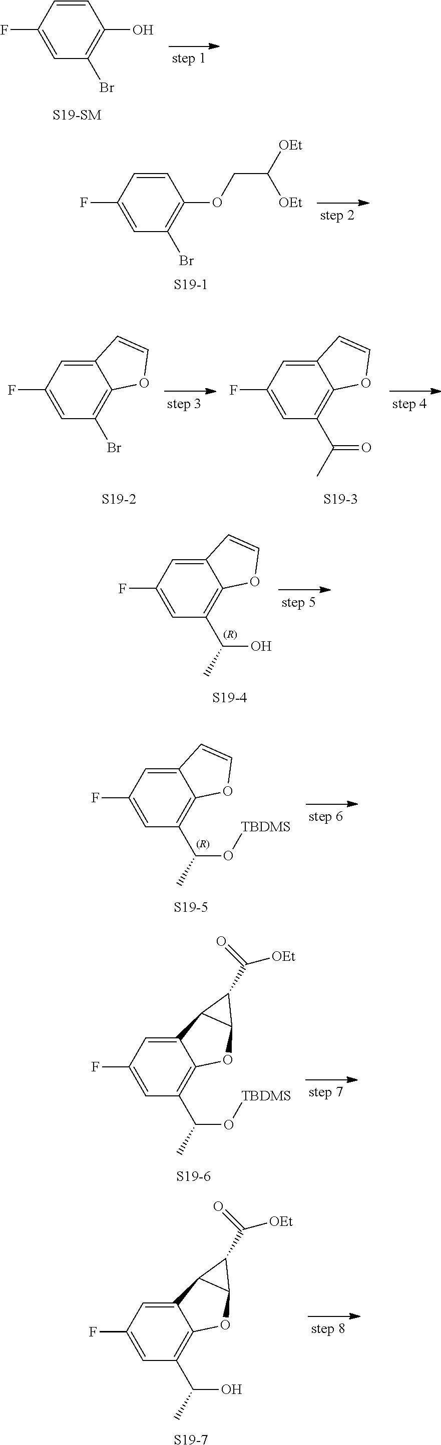

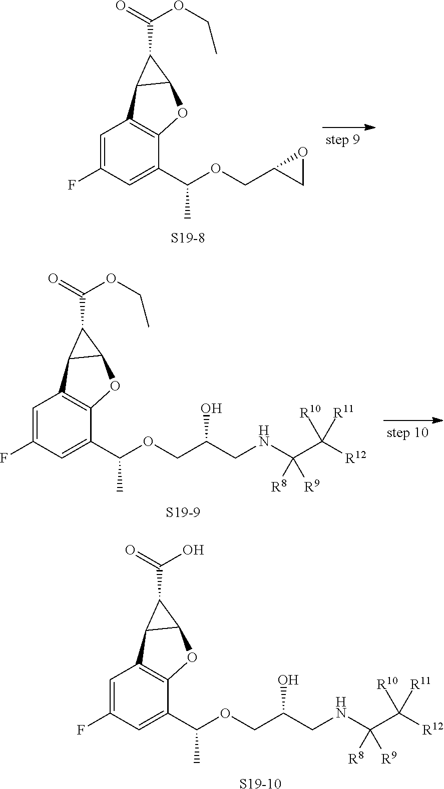

### Experimental Procedure

### Step 1

To a solution of 2-bromo-4-fluorophenol (S19-SM, 50 g, 0.26 mol) and 2-bromo-1,1-diethoxyethane (129 g, 0.65 mol) in DMF was added K₂CO₃ (72 g, 0.52 mol) in portion. The mixture was stirred at 120° C. for 3 hrs. After cooling, the mixture was poured into water, and extracted with ethyl acetate. The organic layer was washed with brine, separated, dried over anhydrous sodium sulfate, filtered and concentrated to obtain S19-1, which was purified by column chromatography (petroleum:ethyl acetate=20:1).

### Step 2

A mixture of AMBERLYST 15 ion exchange resin (25 g) in chlorobenzene (150 mL) was heated at reflux to remove water by azeotropic distillation. Then to this mixture, a solution of S19-1 (25 g, 0.08 mol) in chlorobenzene (1000 mL) was added dropwise over 2 hrs. The mixture was stirred at reflux with constant removal of water. Then the mixture was cooled to room temperature. The filtered cake was washed with dichloromethane (200 mL) and the combined filtrate was concentrated to obtain S19-2, which was purified by column chromatography (eluted with petroleum).

### Step 3

To a solution of S19-2 (5 g, 0.023 mol), 1,3-bis(diphenylphosphino) propane (0.67 g, 1.6 mmol), palladium acetate (175 mg, 0.78 mmol) in ethyleneglycol (20 mL) were added Et₃N (4.85 g) and 1-(vinyloxy)butane (7.4 g, 0.074 mol) under N₂. The mixture was stirred at 120° C. for 4 hrs. TLC indicated the completion of the reaction. After the mixture was cooled, aq. 1N HCl was added dropwise to make the PH 3-4. After stirring for 2 hrs, the mixture was diluted with ethyl acetate (200 mL) and washed with brine. The organic layer was separated, dried over anhydrous sodium sulfate, filtered and concentrated to provide a residue, which was purified by column chromatography (eluted with petroleum:ethyl acetate=20:1) to afford 519-3.

### Step 4

(S)-diphenyl prolinol (1.5 g, 8.4 mmol) was added to anhydrous tetrahydrofuran, then trimethyl borate (1.05 g, 10 mmol) was added to the solution at 0° C. ˜−10° C. under N₂. The mixture was stirred at room temperature overnight. A solution of borane dimethyl sulfide complex (2.0 M in THF, 17 mL, 34 mmol) in THF were added to it. Then a solution of S19-3 in THF was added by syringe pump at 0° C. ˜−10° C. over 5 hrs. TLC indicated the completion of the reaction. The reaction was quenched with diluted HCl (2N), and diluted with ethyl acetate. The organic layer was washed with aq. NaHCO₃ and brine. The organic layer was separated, dried over anhydrous sodium sulfate, filtered and concentrated to provide a residue, which was purified by column chromatography to afford S19-4.

### Step 5

To a solution of S19-4 (3.0 g, 0.017 mol) in DMF (50 mL) was added imidazole (2.8 g, 0.042 mol), followed by tert-butyldimethylchlorosilane (TBDMSCl, 3.0 g, 0.02 mol) at 0° C. The mixture was stirred at room temperature overnight. Then the reaction solution was diluted with ethyl acetate and washed with brine. The organic layer was separated, dried over anhydrous sodium sulfate, filtered and concentrated to provide a residue, which was purified by column chromatography to afford S19-5.

### Step 6

Copper (I) triflate (2:1 complex with toluene, 320 mg, 4%) and (R,R)-(+)-2,2-Isopropylidenebis(4-tert-butyl-2-oxazoline) (0.23 g, 5%, DL Chiral Chemicals) were stirred in dichloromethane (20 mL) at room temperature under N₂ atmosphere for overnight. A drop of ethyl diazoethanoate was added to this deep green solution. The color temporarily faded to brown and gas evolving was observed. A solution of S19-5 (4.5 g, 0.015 mol) in dichloromethane (100 mL) was added, followed by a slow addition of a solution of ethyl diazoethanoate (9.3 mL, 0.06 mol) in DCM (40 mL) during a period of 16 hrs using a syringe pump. The reaction was stirred at room temperature for 2 hrs after the addition. The mixture was concentrated and the residue was purified by column chromatography to afford S19-6, which was directly used in the next step.

### Step 7

Tetrabutylammonium fluoride (11.0 g, 0.04 mol) was added to a solution of S19-6 (8.0 g, 0.032 mol) in THF (150 mL) at 0° C. The reaction was stirred at room temperature for 6 hrs. The mixture was diluted with ethyl acetate and washed with brine. The organic layer was separated, dried over anhydrous sodium sulfate, filtered and concentrated to provide a residue, which was purified by column chromatography to afford 519-7.

### Step 8

To a mixture of S19-7 (2.2 g, 8.3 mmol) and (R)-oxiran-2-ylmethyl 3-nitrobenzenesulfonate (4.3 g, 16.5 mmol) in DMF (50 mL) was added NaH (0.41 g, 16.5 mmol) at 0° C. The reaction was stirred at room temperature for 2 days. The mixture was diluted with ethyl acetate and washed with brine. The organic layer was separated, dried over anhydrous sodium sulfate, filtered and concentrated to provide a residue, which was purified by column chromatography to afford S19-8.

### Step 9

To a solution of S19-8 (200 mg, 0.62 mmol) in DMF (2 mL) was added amine (0.93 mmol). The solution was stirred at 85° C. overnight. Then the reaction solution was diluted with ethyl acetate. The organic layer was washed with brine, separated, dried over anhydrous sodium sulfate, filtered and concentrated to provide a residue, which was purified by prep-TLC to afford S19-9.

### Step 10

2N aq. NaOH (0.5 mL) was added to S19-9 (0.21 mmol) in MeOH (1.0 mL) and THF (1.0 mL). The solution was stirred at 60° C. for 0.5 hrs. Then MeOH and THF were removed under reduced pressure. 1N aq. HCl was added to the reaction in water (10 mL) to make PH=7. The precipitate was collected and purified by prep-HPLC to afford the product S19-10.

The following compound in Table 19 was made according to EXAMPLE 19.

TABLE 19

| | | FLIPR | |
|---|---|---|---|
| Exam- | | ASSAY | |
| ple | Structure | IC₅₀ (nM) | Analysis Data |
| 19-1 | 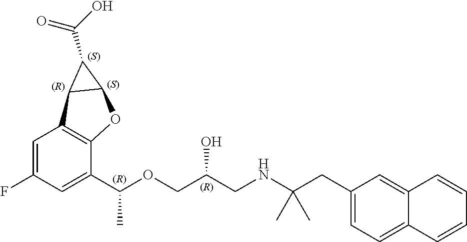 | 176 | ¹H NMR (MeOD, 300 MHz) δ 7.91-7.81 (m, 3H), 7.79-7.75 (m, 1H), 7.53-7.46 (m, 2H), 7.45-7.34 (m, 1H), 7.23-7.19 (m, 1H), 7.05-6.92 (m, 1H), 5.25-5.19 (m, 1H), 4.46-4.38 (m, 1H), 4.04-3.91 (m, 1H), 3.51-3.43 (m, 1H), 3.39-3.33 (m, 2H), 3.22-3.04 (m, 4H), 1.40 (s, 6H), 1.35-1.30 (m, 3H), 1.23 (s, 1H). LC-MS (ESI): 494.2 [M + 1]. HPLC (220 nm): 97.50%. |
| 19-2 | 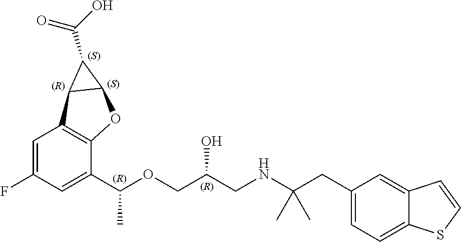 | N/A | ¹H NMR (MeOD, 300 MHz) δ 7.97-7.91 (m, 1H), 7.80 (s, 1H), 7.69-7.58 (m, 1H), 7.45-7.38 (m, 1H), 7.33-7.24 (m, 2H), 7.09-6.98 (m, 1H), 5.35-5.13 (m, 1H), 4.55-4.35 (m, 1H), 4.14-3.95 (m, 1H), 3.57-3.44 (m, 1H), 3.42-3.37 (m, 2H), 3.33-3.28 (m, 1H), 3.20-3.05 (m, 3H), 1.42-1.33 (m, 9H), 1.27 (s, 1H). LC-MS (ESI): 500.2 [M + 1]. HPLC (220 nm): 95.46%. |
| 19-3 | 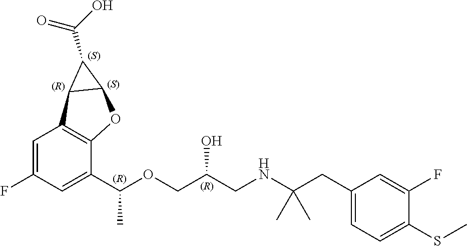 | N/A | ¹H NMR (MeOD, 300 MHz) δ 7.42-7.30 (m, 1H), 7.30-7.20 (m, 1H), 7.12-6.98 (m, 1H), 5.25-5.03 (m, 1H), 4.50-4.40 (m, 1H), 4.03-3.90 (m, 1H), 3.50-3.40 (m, 2H), 3.30-2.20 (m, 1H), 3.15-2.90 (m, 4H), 2.45 (s, 3H), 1.40-1.32 (d, 3H), 1.28 (s, 6H), 1.22 (s, 1H). LC-MS (ESI): 508.2 [M + 1]. HPLC (220 nm): 95.17%. |

### Example 20

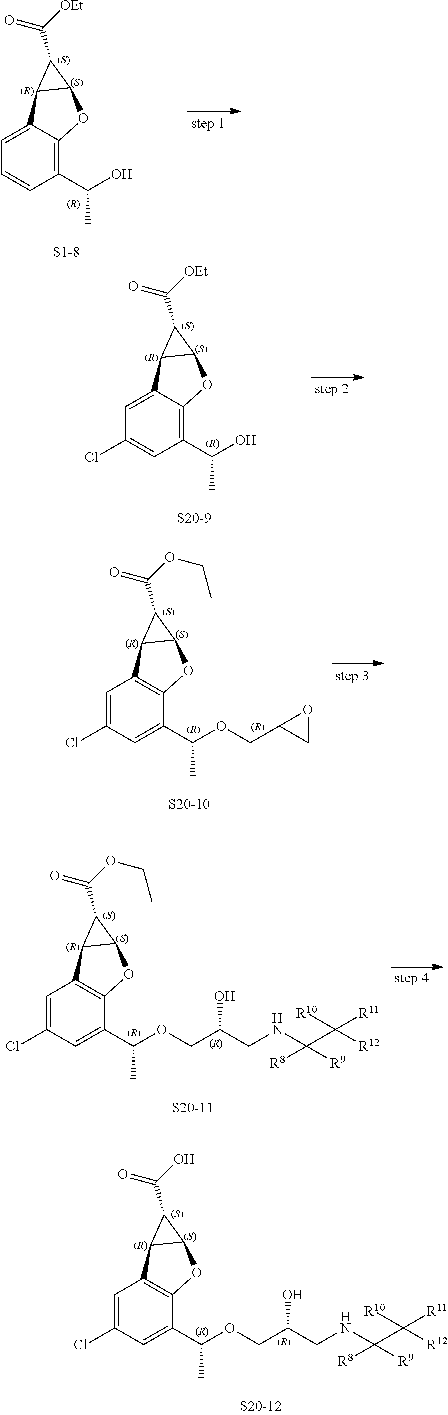

### Experimental Procedure

### Step 1

To a mixture of S1-8 in 2-propanol (150 mL) was added N-Chlorosuccinimide (13.6 g, 100 mmol). The reaction mixture was stirred at 50° C. for 48 hrs. It was concentrated, filtered and washed with ethyl acetate. The filtrates were washed with brine. The organic layer was separated, dried over anhydrous sodium sulfate, filtered and concentrated to provide a residue, which was purified by column chromatography to afford S20-9.

### Step 2

To a mixture of S20-9 (1 g, 3.5 mmol) and (R)-oxiran-2-ylmethyl 3-nitrobenzenesulfonate (1.8 g, 7 mmol) in DMF (15 mL) was added NaH (177 mg, 7 mmol) at 0° C. The reaction was stirred at room temperature for 2 days. The mixture was diluted with ethyl acetate and washed with brine. The organic layer was separated, dried over anhydrous sodium sulfate, filtered and concentrated to provide a residue, which was purified by column chromatography to afford S20-10.

### Step 3

To a solution of S20-10 (205 mg, 0.6 mmol) in DMF (2 mL) was added amine (0.9 mmol). The solution was stirred at 85° C. overnight. Then the reaction solution was diluted with ethyl acetate and washed with brine. The organic layer was separated, dried over anhydrous sodium sulfate, filtered and concentrated to provide a residue, which was purified by prep-TLC to afford S20-11.

### Step 4

2N aq. NaOH (0.5 mL) was added to S20-11 (0.207 mmol) in MeOH (1.0 mL) and THF (1.0 mL). The solution was stirred at 60° C. for 0.5 hrs. Then MeOH and THF were removed under reduced pressure. 1N aq. HCl was added to the reaction in water (10 mL) to make PH=7. The precipitate was collected and purified by prep-HPLC to afford the product S20-12.

The following compounds in Table 20 were made according to EXAMPLE 20.

TABLE 20

| | | FLIPR | |
|---|---|---|---|
| Exam- | | ASSAY | |
| ple | Structure | IC₅₀ (nM) | Analysis Data |
| 20-1 | 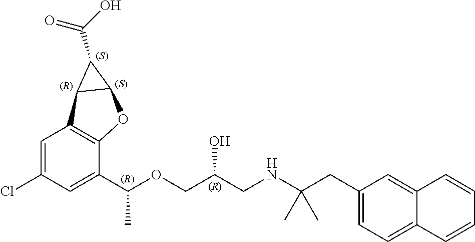 | 20 | ¹H NMR (MeOD, 300 MHz) δ 7.91-7.82 (m, 2H), 7.80-7.73 (m, 1H), 7.54-7.43 (m, 2H), 7.42-7.38 (m, 2H), 7.21-7.19 (s, 1H), 5.20-5.05 (m, 1H), 4.75-4.60 (m, 1H), 4.12-3.85 (m, 1H), 3.58-3.45 (m, 1H), 3.30-3.20 (m, 2H), 3.10-3.02 (m, 4H), 1.35 (s, 6H), 1.30-1.29 (d, 3H), 1.20 (s, 1H). LC-MS (ESI) 510.1 [M + 1]. HPLC (220 nm): 95.34%. |
| 20-2 | 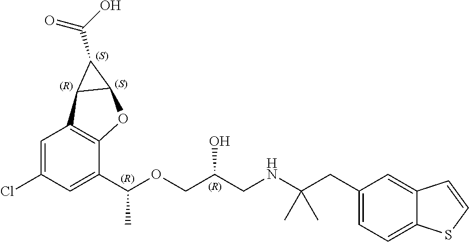 | N/A | ¹H NMR (MeOD, 300 MHz) δ 7.92-7.88 (m, 1H), 7.75 (s, 1H), 7.60-7.56 (m, 1H), 7.40-7.37 (m, 2H), 7.30-7.20 (m, 2H), 5.20-5.18 (m, 1H), 4.68-4.60 (m, 1H), 4.07-3.96 (m, 1H), 3.58-3.36 (m, 3H), 3.20-3.04 (m, 4H), 1.42-1.25 (m, 9H), 1.22-1.20 (m, 1H). LC-MS (ESI): 516.2 [M + 1]. HPLC (220 nm): 96.69%. |

### Example 21

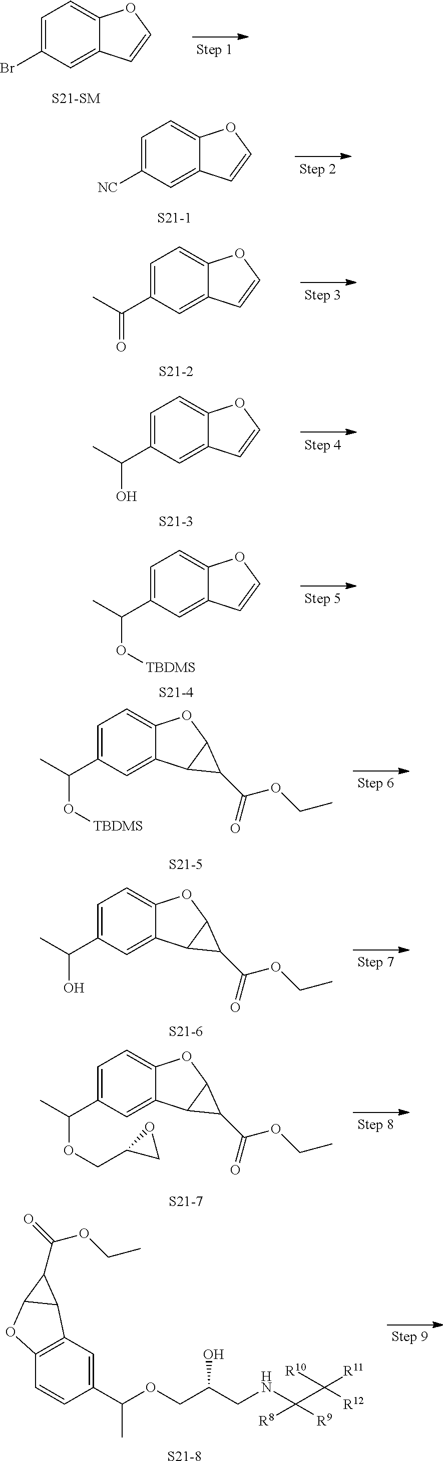

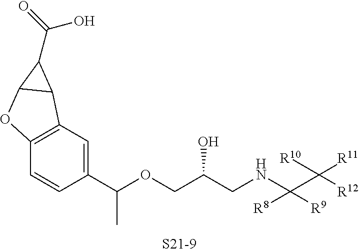

### Experimental Procedure

### Step 1

A mixture of S21-SM (19.6 g, 0.1 mol), CuCN (27.3 g, 0.3 mol) and CuI (38.3 g, 0.2 mol) in DMF (200 mL) was stirred at 150° C. for 4 hs. Cooled, the mixture was diluted with ethyl acetate and washed with brine, dried over anhydrous sodium sulfate, filtrated and concentrated to provide a residue, which was purified by column chromatography to afford S21-1.

### Step 2

To a mixture of S21-1 (20.0 g, 0.14 mol) in toluene (150 mL) was added dropwise methylmagnesium bromide (140 mL, 3.0 M, 0.42 mol) at RT under N₂. The solution was stirred at 60° C. for 1 h. NH₄Cl (aq.) was added to the solution and the mixture was acidified with 1N HCl. The resulting mixture was refluxed for 1 h, and it was extracted with ether, washed with NaHCO₃ and brine, dried over anhydrous sodium sulfate, filtrated and concentrated to provide a residue, which was purified by column chromatography to afford S21-2.

### Step 3

To a solution of S21-2 (3.2 g, 0.02 mol) in MeOH (40 mL) was added NaBH₄ (1.52 g, 0.04 mol). The mixture was stirred at RT for 2 hs. The reaction mixture was concentrated, extracted with ethyl acetate, washed with brine, dried over anhydrous sodium sulfate, filtrated and concentrated to provide a residue, which was purified by column chromatography to afford S21-3.

### Step 4

To a solution of S21-3 (3.1 g, 0.019 mol) in DMF (50 mL) was added imidazole (3.25 g, 0.048 mol), followed by tert-butyldimethylchlorosilane (TBDMSCl, 3.4 g, 0.023 mol) at 0° C. The mixture was stirred at RT for 2 hs. Then the reaction solution was extracted with ethyl acetate, washed with brine, dried over anhydrous sodium sulfate, filtrated and concentrated to provide a residue, which was purified by column chromatography to afford S21-4.

### Step 5

Copper (I) triflate (2:1 complex with toluene, 95 mg, 0.36 mmol) and (R,R)-(+)-2,2-Isopropylidenebis(4-tert-butyl)-2-oxazoline (0.135 g, 0.46 mmol, DL Chiral Chemicals) were stirred in dichloromethane (20 mL) at RT under N₂ atmosphere for 1.5 hs. A drop of ethyl diazoethanoate was added to this deep green solution. The color temporarily faded to brown and gas evolving was observed. A solution of S21-4 (5.0 g, 0.018 mol) in dichloromethane (80 mL) was added, followed by a slow addition of a solution of ethyl diazoethanoate (11 mL, 0.09 mol) in DCM (40 mL) during a period of 16 hs using a syringe pump. The reaction was stirred at RT for 2 hs after the addition. The mixture was concentrated and purified by column chromatography to afford crude S21-5.

### Step 6

Tetrabutylammonium fluoride (20 mL, 1 M in THF, 0.02 mol) was added dropwise to a solution of S21-5 (5.0 g, 0.014 mol) in THF (50 mL) at 0° C. The reaction was stirred at RT for 6 hs. Filtered and washed with ethyl acetate. The filtrate was washed with brine, dried over anhydrous sodium sulfate, filtrated and concentrated to provide a residue, which was purified by column chromatography to afford S21-6.

### Step 7

To a mixture of S21-6 (1.0 g, 0.004 mol) and (R)-oxiran-2-ylmethyl 3-nitrobenzenesulfonate (1.25 g, 0.005 mol) in DMF (10 mL) was added NaH (0.16 g, 0.004 mol) at 0° C. The reaction was stirred at RT for 2 days. The mixture was diluted with ethyl acetate and washed with brine, dried over anhydrous sodium sulfate, filtrated and concentrated to provide a residue, which was purified by column chromatography to afford S21-7.

### Step 8

To a solution of S21-7 (0.3 mmol) in DMF (2 mL) was added amine (0.35 mmol) at room temperature. The solution was stirred overnight. Then the reaction solution was extracted with ethyl acetate. The combined organic layers were washed with brine, dried over anhydrous sodium sulfate, filtrated and concentrated to provide a residue, which was purified by prep_TLC to afford S21-8.

### Step 9

2N aq. NaOH (0.5 mL) was added to S21-8 (0.207 mmol) in MeOH (1.0 mL) and THF (1.0 mL). The solution was stirred at 60° C. for 0.5 hrs. Then MeOH and THF was removed under reduced pressure. 1N aq. HCl was added to the reaction in water (10 mL) to make PH=7. The precipitate was collected and purified by pre-HPLC to afford S21-9.

The following compounds in Table 21 were made according to EXAMPLE 21.

TABLE 21

| | | FLIPR | |
|---|---|---|---|
| Exam- | | ASSAY | |
| ple | Structure | IC₅₀ (nM) | Analysis Data |
| 21-1 | 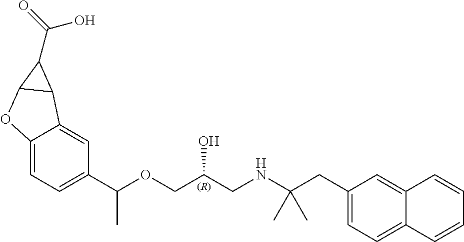 | 2722 | ¹H NMR (MeOD, 300 MHz) δ 7.80-7.70 (m, 2H), 7.66-7.60 (m, 1H), 7.40-7.30 [symbol] 7.30-7.12 (m, 2H), 7.10-6.90 (m, 1H), 6.90-6.80 (m, 1H), 6.65-6.60 (m, 1H), 4.95- [symbol] 1H), 4.35-4.20 (m, 1H), 3.95-3.75 (m, 1H), 3.50-3.20 (m, 3H), 3.08-3.02 (m, 1 [symbol] 2.80 (m, 3H), 2.22 (s,1H), 1.15-1.05 (m, 9H). LC-MS (ESI) 476.0 [M + 1]. HPLC ( [symbol] 100.00%. |
| 21-2 | 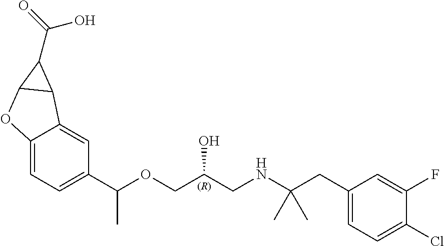 | N/A | LC-MS (ESI) 477.9 [M + 1] |
| 21-3 | 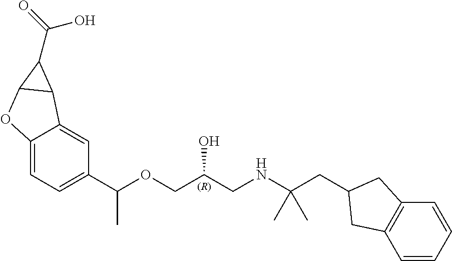 | N/A | LC-MS (ESI) 466.0 [M + 1] |

[symbol]

indicates data missing or illegible when filed

### Example 22

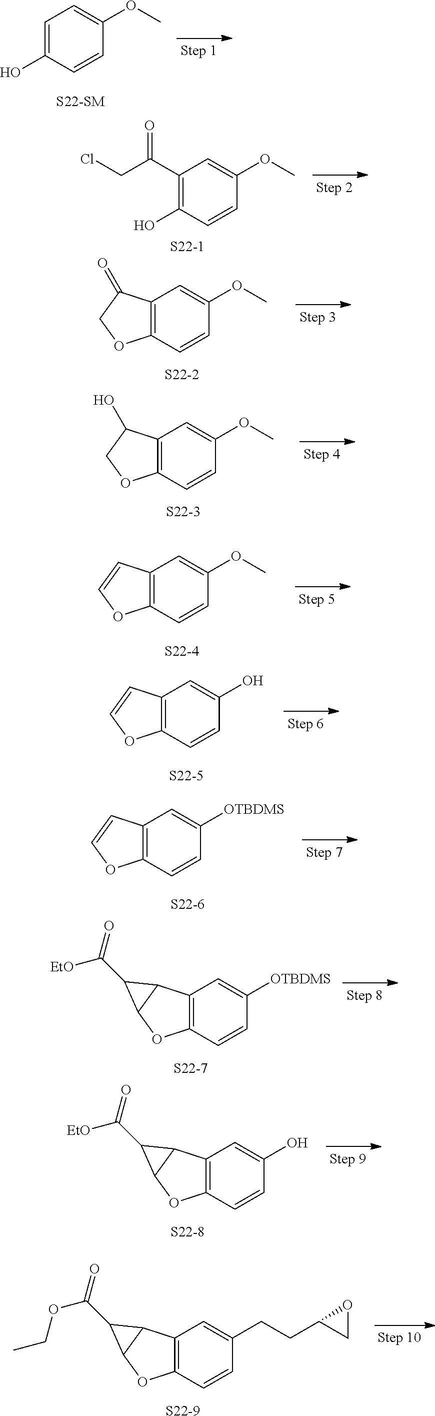

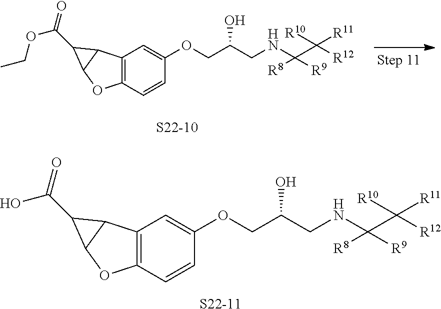

### Step 1

To an ice cooled solution of boron trichloride (1 M in methylene chloride, 194 mL) was added a solution of S22-SM (20 g, 160 mmol) in 1,2-dichloroethane (80 mL) dropwise over 10 min. To the resulting mixture was added sequentially chloroacetonitrile (12.3 mL, 192 mmol) dropwise over 2 min and solid aluminum chloride (10.7 g, 80 mmol) in portions such that the reaction temperature did not exceed 35° C. The reaction mixture was allowed to stir for 2.5 h and then poured into a mixture of ice and 2 N HCl (100 mL). The layers were separated, the aqueous layer was extracted with methylene chloride, and the combined organic extracts were dried (MgSO₄,) and concentrated to afford S22-1.

### Step 2

To a solution of S22-1 (32 g, 160 mmol) in methanol (160 mL) was added sodium acetate (47 g). The mixture was refluxed for 1.5 h, allowed to cool, and filtered. The filtrate was poured into 5% aq. NaCl (400 mL). The red solid that precipitated was collected by filtration, dried, and recrystallized from ether to afford S22-2.

### Step 3

To the solution of S22-2 (5.1 g, 31 mmol) in CH₃OH (100 mL) at 0° C. was added NaBH₄ (4.7 g, 124 mmol). The mixture was stirred at r.t. overnight. Solvents were removed from the system. KOH (20%) was added and stirred for 20 min. The organic layer was separated, dried and concentrated to afford S22-3.

### Step 4

To the solution of S22-3 (5 g, 30 mmol) in acetonitrile (200 mL) was added trifluoroacetic acid (2 mL). NaHCO₃ was added to quench the reaction and extracted by ethyl acetate. After washed with brine, the organic was dried over Na₂SO₄ and concentrated. The residue was purified by silica gel chromatography to afford S22-4.

### Step 5

BBr₃ (2.6 mL, 13.5 mmol) was added dropwise to S22-4 (2.0 g, 13.5 mmol) in DCM (20 mL) at −60° C. under N₂. The solution was stirred at 0° C. for 2 h. The mixture was poured into ice and adjusted the pH to 12˜13. The inorganic phase was extracted with DCM. The combined organic layers were washed with brine, dried over anhydrous sodium sulfate, filtrated and concentrated to provide a residue, which was purified by column chromatography to afford S22-5.

### Step 6

To the solution of S22-5 (4 g, 30 mmol) and imidazol (4.1 g, 60 mmol) in DMF (100 mL) was added TBDMSCl (5.4 g, 36 mmol) at 0° C. under N₂. The mixture was stirred at r.t. overnight. Water was added to the reaction and the mixture was extracted with ethyl acetate. The residue was purified by silica gel chromatography to afford S22-6.

### Step 7

To a refluxing solution of S22-6 (500 mg, 2.0 mmol) and Copper(II) acetylacetonate (53 mg, 0.2 mmol) in dichloroethane (30 mL) was added ethylenediamine (2.3 g, 20 mmol) with a syringe pump (1 eq/h). Once addition was complete (5 h), the solution was allowed to stir and reflux for additional 30 min. Water was added to quench the reaction and the mixture was extracted with DCM. The organic layer was separated, dried and concentrated. The residue was purified by silica gel chromatograph to afford S22-7.

### Step 8

To the solution of S22-7 (5 g, 15 mmol) in THF (40 mL) was added tetrabutylammonium fluoride (1 mol/L, 40 mL, 15 mmol). The mixture was stirred at r.t. overnight. Water was added to quench the reaction, and the mixture was extracted with ethyl acetate. The organic layer was separated, dried and concentrated. The residue was purified by silica gel chromatography to afford S22-8.

### Step 9

To a solution of S22-8 (1.1 g, 5 mmol) in DMF (20 mL) was added CsF (0.15 g, 1 mmol) and K₂CO₃ (0.83 g, 6 mmol). The mixture was stirred for 1 h at room temperature and then (R)-(−)-glycidyl-3-nitrobenzenesulfonate (1.4 g, 5.5 mmol) was added. The reaction mixture was stirred at room temperature overnight. Then the reaction was extracted with ethyl acetate (60 mL×2). The combined organic layers were washed with brine, dried over anhydrous sodium sulfate, filtrated and concentrated to provide a residue, which was purified by column chromatography to afford S22-9.

### Step 10

To a solution of S22-9 (0.76 mmol) in ethanol (2 mL) was added amine (1.5 mmol) at room temperature. The solution was stirred at reflux overnight. The solvents were removed from the system. Ethyl acetate was added and the mixture was washed with brine, dried over anhydrous sodium sulfate, filtrated and concentrated to provide a residue, which was purified by prep_TLC to afford S22-10.

### Step 11

2N aq. NaOH (0.5 mL) was added to S22-10 (0.21 mmol) in a mixture of MeOH (1.0 mL) and THF (1.0 mL). The solution was stirred at 60° C. for 2 h. Then MeOH and THF was removed under reduced pressure. 1N aq. HCl was added to the reaction in water (10 mL) to make pH=7. The mixture was filtered to get S22-11.

The following compounds in Table 22 were made according to EXAMPLE 22.

TABLE 22

| | | FLIPR | |
|---|---|---|---|
| Exam- | | ASSAY | |
| ple | Structure | IC₅₀ (nM) | Analysis Data |
| 22-1 | 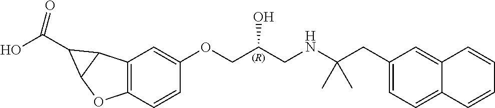 | 3551 | ¹H NMR (301 MHz, CD₃OD) δ 7.85 (t, J = 7.3 Hz, 2H), 7.79 (s, 1H), 7.49 (dd, J = 6.2, 3.2 Hz, 1H), 7.41 (d, J = 7.0 Hz, 1H), 7.00 (d, J = 3.4 Hz, 1H), 6.77 (d, J = 8.8 Hz, 1H), 5.06 (d, J = 5.5 Hz, 1H), 4.22 (s, 1H), 4.02 (dd, J = 12.6, 7.6 Hz, 2H), |
| | | | 3.49-3.38 (m, 1H), 3.25-3.09 |
| | | | (m, 3H), 1.41 (s, 6H), 1.10 (s, 1H). |
| 22-2 | 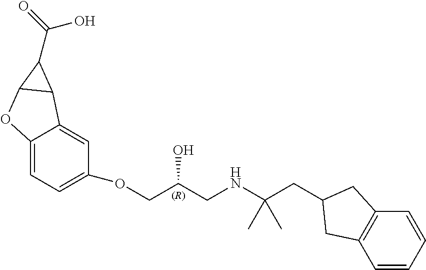 | N/A | ¹H NMR (301 MHz, CD₃OD) δ 7.20-7.04 (m, 5H), 6.79 (s, 2H), 5.06 (d, J = 5.4 Hz, 1H), 4.18 (s, 1H), 4.00 (s, 2H), 3.26 (dd, J = 5.5, 3.1 Hz, 1H), 3.15 (dd, J = 14.4, 6.8 Hz, 3H), 2.77-2.61 (m, 2H), 2.61-2.45 (m, 1H), 2.03 (d, J = 6.0 Hz, 2H), 1.48 (s, 6H), 1.10 (d, J = 3.0 Hz, 1H). LC-MS (ESI) 438.0 [M + 1] |
| 22-3 | 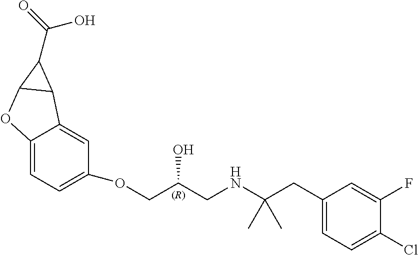 | N/A | ¹H NMR (301 MHz, CD₃OD) δ 7.46 (t, J = 8.0 Hz, 1H), 7.22 (d, J = 10.2 Hz, 1H), 7.11 (s, 2H), 6.79 (d, J = 1.4 Hz, 2H), 5.07 (d, J = 5.4 Hz, 1H), 4.21 (s, 1H), 4.02 (s, 2H), 3.40 (d, J = 12.9 Hz, 1H), 3.26-3.12 (m, 2H), 3.05 (s, 2H), 1.36 (s, 6H), 1.11 (d, J = 2.9 Hz, 1H). LC-MS (ESI) 450.0 [M + 1] |

### Example 23

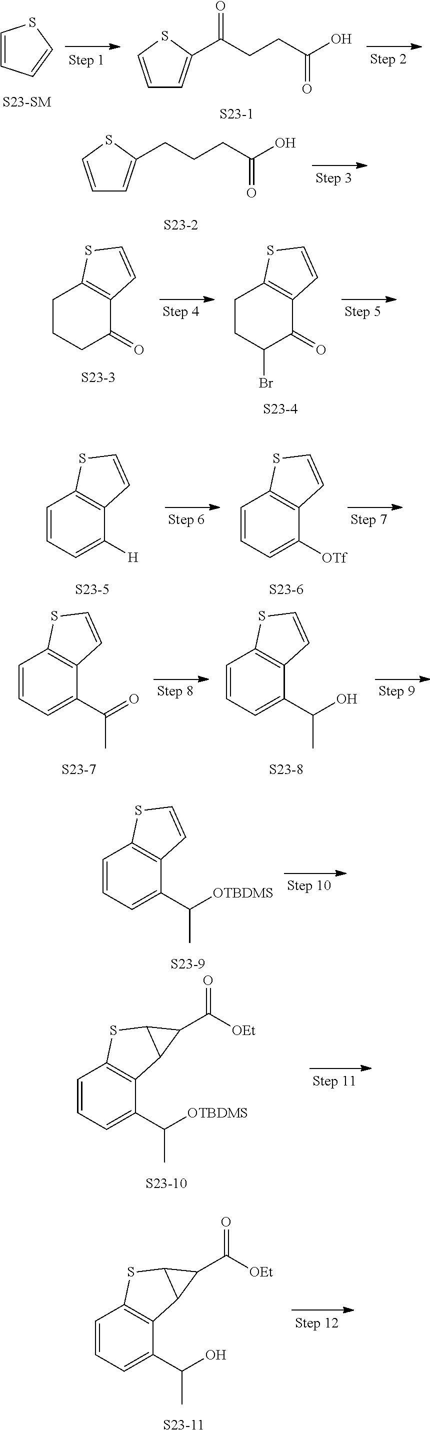

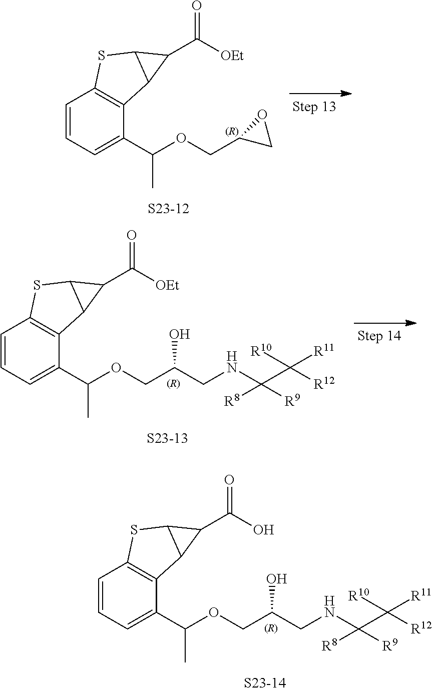

### Step 1: S23-SM

(28.5 mL, 0.36 mol) was dissolved in anhydrous DCM (170 mL) and the solution was added to a suspension of dihydrofuran-2,5-dione (35.7 g, 0.36 mol) and AlCl₃ (104 g, 0.36 mol) in anhydrous DCM (350 mL) at room temperature. The reaction was stirred over night. The mixture was added to a solution of 250 mL of cold HCl and 200 m/1 of cold water at 0° C. The mixture was extracted twice with DCM. The combined organic layers were washed with NaOH (4N aq., 500 mL) solution, and extracted with DCM. After acidification with HCl (4N aq.), the aqueous phase was extracted with DCM, dried over Na₂SO₄, concentrated to afford S23-1.

### Step 2

Hydrazine (80%, 50 mL, 1.04 mol) and KOH (53 g, 0.98 mol) were added to a solution of S23-1 (52 g, 0.28 mol) in diethylene gloycol (600 mL). Then the mixture was heated for 7 h at 195° C. Diluted at room temperature with 2.5 L of water, and extracted with ethyl acetate (800 mL). The resulting aqueous phase was acidified (4N aq. HCl). Then it was extracted with ethyl acetate. The organic layer was dried over Na₂SO₄, and concentrated to afford S23-2.

### Step 3

A mixture of S23-2 (44 g, crude) in 63 mL of acetic anhydride containing 1.3 mL of H₃PO₄ was stirred at 120° C. for 3 h. After cooled, the mixture was treated with 350 mL of water and the mixture was extracted with DCM. The organic layer was washed with a solution of NaOH until pH=7, dried over anhydrous sodium sulfate, filtrated and concentrated to provide a residue, which was purified by column chromatography to afford S23-3.

### Step 4

To a mixture of S23-3 (30 g, 0.2 mol) in CCl₄ (1300 mL) was added dropwise bromine (10 mL, 0.2 mol) containing a few drops of Et₂O at −10° C. The solution was stirred at room temperature over night. After concentrated, the mixture was extracted with ethyl acetate, washed with saturated aq. NaHCO₃ and brine, dried over anhydrous sodium sulfate, filtrated and concentrated to provide S23-4.

### Step 5

A solution of S23-4 (50 g, crude) and Li₂CO₃ (24 g, 0.44 mol) and LiBr (32 g, 0.49 mol) in DMF was refluxed for 3 h. The mixture was diluted with ethyl acetate, washed with HCl (2N aq.) and brine, dried over anhydrous sodium sulfate, filtrated and concentrated to provide a residue, which was purified by column chromatography to afford S23-5.

### Step 6

To a solution of S23-5 (20 g, 0.13 mol) and 2,6-lutidine (39 mL) in dry DCM (200 mL), trifluoromethanesulfonic anhydride (33.8 mL, 0.20 mol) in dry DCM (100 mL) was added dropwise at −78° C. over 30 min. The reaction mixture was stirred at room temperature for 2 h. The mixture was diluted with ethyl acetate, washed by brine, dried over anhydrous sodium sulfate, filtrated and concentrated to provide a residue, which was purified by column chromatography to afford S23-6.

### Step 7

To a solution of S23-6 (15 g, 53 mmol), Et₃N (21 mL, 159 mmol), Pd(OAc)₂ (0.6 g, 2.7 mmol) and dppp (2.4 g, 5.3 mmol) in ethylene glycol (150 mL), 1-(vinyloxy)butane (21 g, 0.21 mol) was added under a nitrogen atmosphere. Then the reaction was stirred at 60° C. overnight. After cooled to 0° C., 2N aq. HCl was added until pH=3, then the reaction mixture was stirred at 0° C. for 0.5 h. The mixture was extracted with ethyl acetate, washed by brine, dried over anhydrous sodium sulfate, filtrated and concentrated to provide a residue, which was purified by column chromatography to afford S23-7.

### Step 8

To a solution of S23-7 (2.2 g, 12.2 mmol) in MeOH (20 mL), NaBH₄ (0.92 g, 24.4 mmol) was added at 0° C., Then the mixture was stirred at room temperature for 2 h. The reaction mixture was washed by brine, dried over dried over anhydrous sodium sulfate, filtrated and concentrated to provide S23-8.

### Step 9

To a solution of S23-8 (2.1 g, 11.2 mmol) in DMF (30 mL), was added imidazole (2.29 g, 33.6 mmol), followed by tert-butyldimethylchlorosilane (TBDMSCl, 3.37 g, 22.4 mmol) at 0° C. The mixture was stirred at RT for 2 hs. Then the reaction solution was extracted with ethyl acetate, washed with brine, dried over anhydrous sodium sulfate, filtrated and concentrated to provide a residue, which was purified by column chromatography to afford S23-9.

### Step 10

To a solution of S23-9 (1.0 g, 3.4 mmol) and CuBr (0.48 g, 3.4 mmol) in 1,2-dichloroethane (10 mL), a solution of ethyl diazoethanoate (1.5 g, 13.6 mmol) in 1,2-dichloroethane (5 mL) was added during a period of 8 h using a syringe pump. The reaction was stirred at 80° C. overnight. The mixture was concentrated and purified by column chromatography to afford S23-10.

### Step 11

Tetrabutylammonium fluoride (TBAF, 1M in THF, 0.06 mol) was added dropwise to a solution of S23-10 (500 mg, 1.32 mmol) in THF (5 mL) at 0° C. The reaction was stirred at RT for 6 h. The mixture was filtered and washed with ethyl acetate. The filtrate was washed with brine, dried over anhydrous sodium sulfate, filtrated and concentrated to provide a residue, which was purified by column chromatography to afford S23-11.

### Step 12

To a mixture of S23-11 (200 mg, 0.75 mmol) and (R)-oxiran-2-ylmethyl 3-nitrobenzenesulfonate (392 mg, 1.5 mmol) in DMF (1 mL) was added NaH (60% w/w, 60 mg, 1.5 mmol) at 0° C. The reaction was stirred at RT for 2 days. The mixture was diluted with ethyl acetate, washed with brine, dried over anhydrous sodium sulfate, filtrated and concentrated to provide a residue, which was purified by column chromatography to afford S23-12.

### Step 13

To a solution of S23-12 (0.19 mmol) in DMF (1 mL) was added amine (0.29 mmol). The solution was stirred at 85° C. overnight. Then the reaction solution was extracted with ethyl acetate. The combined organic layers were washed with brine, dried over anhydrous sodium sulfate, filtrated and concentrated to provide a residue, which was purified by prep-TLC to afford S23-13.

### Step 14

2N aq. NaOH (0.5 mL) was added to S23-13 (0.12 mmol) in MeOH (1.0 mL) and THF (1.0 mL). The solution was stirred at 60° C. for 0.5 hrs. Then MeOH and THF was removed under reduced pressure. 1N aq. HCl was added to the reaction in water (10 mL) to make pH=7. The precipitate was collected and purified by pre-HPLC to afford S23-14.

The following compound in Table 23 was made according to EXAMPLE 23.

TABLE 23

| | | FLIPR | |
|---|---|---|---|
| Exam- | | ASSAY | |
| ple | Structure | IC₅₀ (nM) | Analysis Data |
| 23-1 | 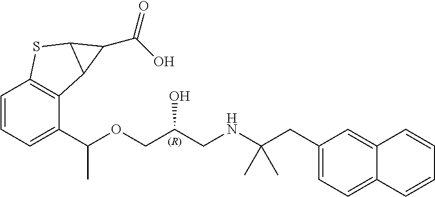 | 42 | ¹H NMR (MeOD, 300 MHz) ¹H NMR (301 MHz, CD3OD) δ 7.86 (d, J = 8.4 Hz, 3H), 7.77 (s, 1H), 7.53-7.47 (m, 2H), 7.40 (d, J = 8.4 Hz, 1H), 7.13 (dd, J = 21.7, 15.8 Hz, 3H), 4.74-4.57 (m, 1H), 4.02 (s, 1H), 3.69 (dd, J = 24.6, 20.3 Hz, 2H), 3.60-3.40 (m, 2H), 3.15 (s, 2H), 3.10 (d, J = 11.1 Hz, 1H), 1.50 (t, J = 6.9 Hz, 2H), 1.42 (d, J = 9.6 Hz, 1H), 1.37 (d, J = 3.5 Hz, 6H), 1.34-1.15 (m, 3H). LC-MS (ESI): 492.2 [M + 1]. HPLC (220 nm): 99.6%. |

### Example 24

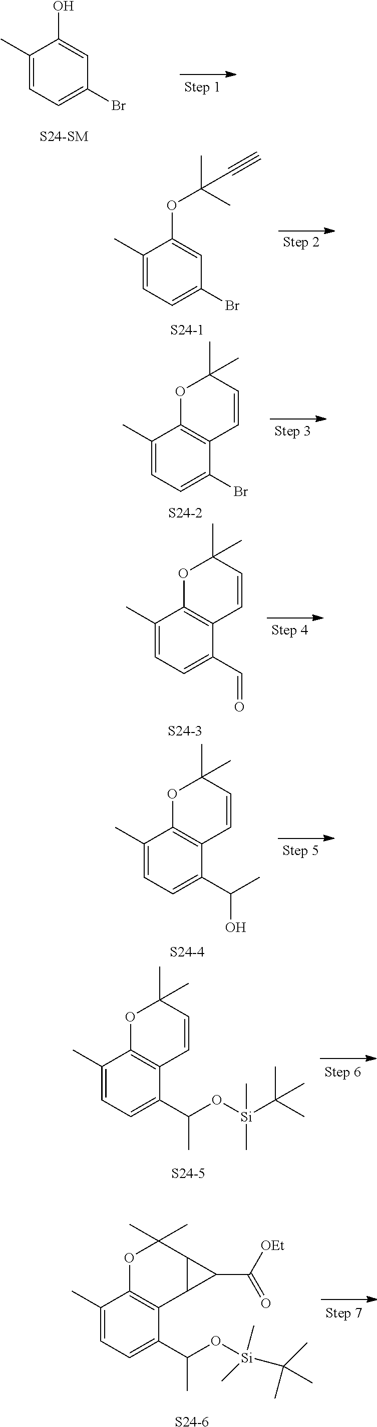

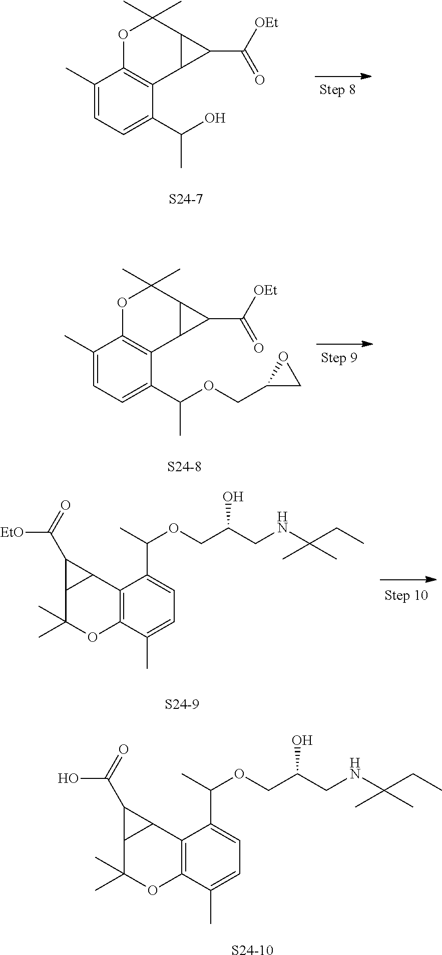

### Step 1

To a solution of 2-methylbut-3-yn-2-ol (10 g, 74 mmol) and 1,8-Diazabicyclo[5.4.0]undec-7-ene (16.8 g, 0.16 mol) in CH₃CN (8 mL) was added dropwise (CF₃CO)₂O (15.4 g, 75 mmol) at 0° C. under N₂. The mixture was stirred at 0° C. for 30 min. Then S24-SM (11.4 g, 61 mmol), 1,8-Diazabicyclo[5.4.0]undec-7-ene (14.5 g, 0.14 mol) and CuCl₂ (50 mg) in CH₃CN (8 mL) was added to the reaction at 0° C. The mixture was stirred overnight. Solvents were removed and the residue was purified by silica gel chromatography to afford S24-1.

### Step 2: S24-1

(9.4 g, 37 mmol) was added into N, N-diethylbenzenamine (40 mL). The mixture was stirred at reflux for 3 hrs. The solution was extracted by ethyl acetate, washed with 1N aq. HCl, aq. NaHCO₃ and brine, dried and concentrated. The residue was purified by silica gel chromatography to afford S24-2.

### Step 3

To a solution of S24-2 (5 g, 19.8 mmol) in THF was added n-BuLi (2.5M in hexanes, 11.9 mL, 29.7 mmol) in THF (50 mL) at −60° C. under N₂ during 20 min. The solution was stirred for 1 h, then DMF (7.2 g, 99 mmol) was added into the system. The solution was warmed to r.t. and stirred overnight. 10% aq. KHSO₄ was added to the solution. The mixture was extracted by ethyl acetate, washed by aq. NaHCO₃ and brine, dried and concentrated. The residue was purified by silica gel chromatography to afford S24-3.

### Step 4

To a solution of S24-3 (500 mg, 2.48 mmol) was added MeLi (1.6M in hexane, 1.23 mL, 1.98 mmol) in THF at −68° C. under N₂. The solution was stirred at r.t. for 1 h. NH₄Cl was added into the reaction and extracted with ethyl acetate. The organic layer was separated, dried and concentrated. The residue was purified by silica gel chromatography to afford S24-4.

### Step 5

To a solution of S24-4 (5 g, 22.9 mmol) in DMF (50 mL) was added imidazole (3.1 g, 46 mmol), followed by tert-butyldimethylchlorosilane (TBDMSCl, 4.1 g, 27.5 mmol) at 0° C. The mixture was stirred at RT for 2 hrs. Then the reaction solution was extracted with ethyl acetate, washed with brine, dried over anhydrous sodium sulfate, filtrated and concentrated to provide a residue, which was purified by column chromatography to afford S24-5.

### Step 6

To a refluxing solution of S24-5 (520 mg, 1.57 mmol) and Copper(II) acetylacetonate (20.6 mg, 0.8 mmol) in dichloroethane (10 mL) was added ethylenediamine (0.9 g, 7.86 mmol) with a syringe pump (1 eq/h). Once the addition was complete (5 hrs), the solution was allowed to stir and reflux for additional 30 min. Water was added to quench the reaction and the mixture was extracted with CH₂Cl₂. The organic layer was separated, dried and concentrated. The residue was purified by silica gel chromatograph to afford S24-6.

### Step 7

Tetrabutylammonium fluoride (TBAF, 20 mL, 1 M in THF, 0.02 mol) was added dropwise to a solution of S24-6 (4.8 g, 0.014 mol) in THF (50 mL) at 0° C. The reaction was stirred at RT for 6 hs. The mixture was filtered and washed with ethyl acetate. The filtrates was washed with brine, dried over anhydrous sodium sulfate, filtrated and concentrated to provide a residue, which was purified by column chromatography to afford S24-7.

### Step 8

To a mixture of S24-7 (0.7 g, 2.3 mmol) and (R)-oxiran-2-ylmethyl 3-nitrobenzenesulfonate (0.66 g, 2.5 mmol) in DMF (10 mL) was added NaH (184 mg, 4.6 mmol) at 0° C. The reaction was stirred at RT for 2 days. The mixture was diluted with ethyl acetate, washed with brine, dried over anhydrous sodium sulfate, filtrated and concentrated to provide a residue, which was purified by column chromatography to afford S24-8.

### Step 9

To a solution of S24-8 (0.30 mmol) in ethanol (2 mL) was added amine (0.60 mmol) at room temperature. The solution was stirred at reflux overnight. The solvents were removed from the system. Ethyl actate was added and the combined organic layers were washed with brine, dried over anhydrous sodium sulfate, filtrated and concentrated to provide a residue, which was purified by prep_TLC to afford S24-9.

### Step 10

2N aq. NaOH (0.5 mL) was added to S24-9 (53.7 mmol) in 1 mL MeOH (1.0 mL) and THF (1.0 mL). The solution was stirred at 60° C. for 2 h. Then MeOH and THF was removed under reduced pressure. 1N aq. HCl was added to the reaction in water (10 mL) to make pH=7. The mixture was filtered to S24-10.

The following compounds in Table 24 were made according to EXAMPLE 24.

TABLE 24

| | | FLIPR | |
|---|---|---|---|
| Exam- | | ASSAY | |
| ple | Structure | IC₅₀ (nM) | Analysis Data |
| 24-1 | 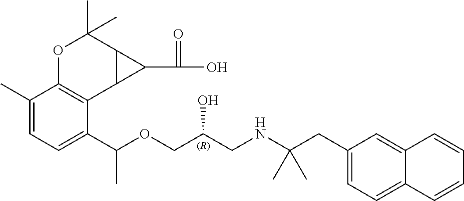 | 106 | ¹H NMR (MeOD, 300 MHz) δ 7.85~7.73 (m, 4H), 7.48~7.45 (m, 3H), 7.17~7.11 (m, 3H), 6.91~6.59 (m, 2H), 4.67~4.63 (m, 1H), 4.01~3.87 (m, 1H), 3.46~3.30 (m, 2H), 3.28~2.78 (m, 5H), 2.28 (s, 2H), 2.14~2.01 (m, 4H), 1.99~1.90 (m, 1H), 1.52~1.45 (m, 6H), 1.44~1.25 (m, 7H), 1.05 (s, 3H). LCMS (ESI): 532.3 [M + 1]. HPLC (220 nm): 97.27%. |
| 24-2 | 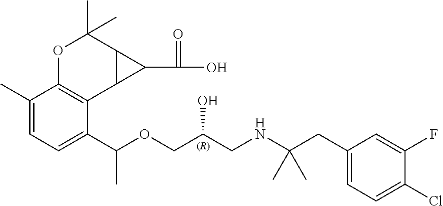 | N/A | ¹H NMR (MeOD, 300 MHz) δ 7.54~7.40 (m, 1H), 7.28~7.19 (m, 1H), 7.18~7.07 (m, 1H), 7.05~6.98 (m, 1H), 6.95~6.80 (m, 1H), 4.88~4.70 (m, 1H), 4.14~3.87 (m, 1H), 3.63~3.41 (m, 1H), 3.19~2.96 (m, 4H), 2.96~2.77 (m, 1H), 2.25~2.16 (m, 1H), 2.15~1.99 (m, 5H), 1.60~1.50 (m, 5H), 1.49~1.40 (m, 2H), 1.40~1.27 (m, 6H), 1.25~1.15 (m, 4H). LCMS (ESI): 534.3 [M + 1]. HPLC (220 nm): 92.46%. |

### Example 25

S25-6 shows calcium sensing receptor antagonist activity. It can be made by the following procedure.

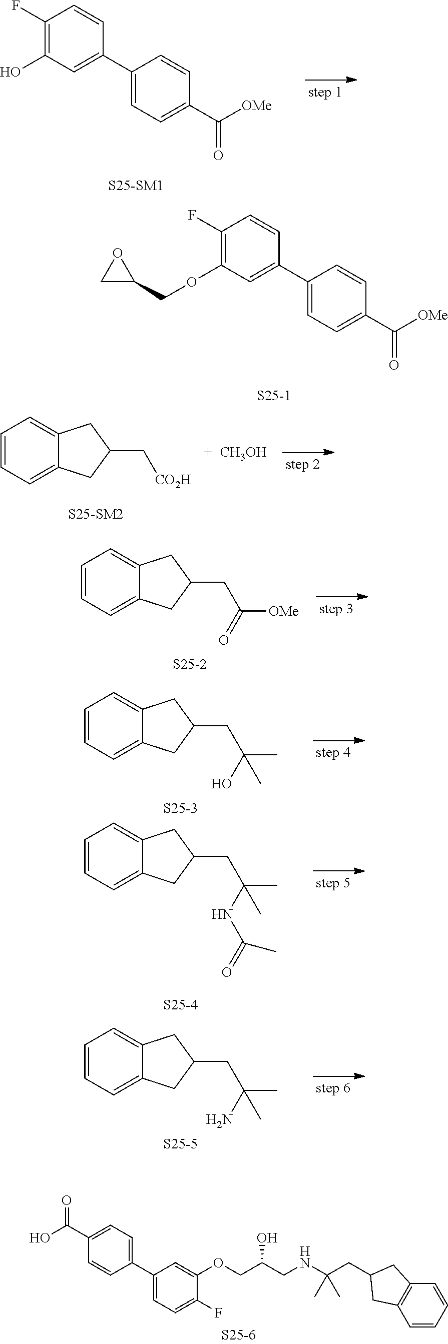

### Step 1

A solution of SM25-SM1 (0.13 mol) and (2R)-glycidyl-3-nitrobenzenesulfonate (0.13 mol) in dry acetone (500 ml) is treated with potassium carbonate (0.26 mol) and the mixture is refluxed under N₂ for 18 h. The reaction is cooled, and filtered. The filtrate is concentrated in vacuo and the residue is purified by flash column chromatography to yield S25-1.

### Step 2

To a solution of S25-SM2(20 g, 0.11 mol) in methanol (200 ml) cooled to 0-10° C. in ice bath is treated dropwise with thionly chloride (14.8 g, 0.125 mol). The mixture is stirred at rt for 16 h, and concentrated in vacuo. The residue is dissolved in ethyl actate, washed with 2.5N sodium hydroxide, water and brine, dried over sodium sulfate, and concentrated in vacuo to give S25-2.

### Step 3

A solution of S25-2 (6.3 g, 33 mmol) in ether (150 ml) is added dropwise to 1.4M methyllithium in ether (100 ml, 4.25 eq) stirred in an ice bath. The mixture is allowed to warm to rt, stirred for 2 h, and quenched by the dropwise addition of saturated aqueous ammonium chloride (150 ml). The aqueous layer is separated and extracted with ether. The combined ether layer is washed with brine, dried over sodium sulfate, and concentrated in vacuo to afford S25-3.

### Step 4

To a mixture of concentrated sulfuric acid (1.7 ml) in acetonitrile (6 ml) in an ice bath is added dropwise a solution of S25-3 (3.3 g, 17.3 mmol) in glacial acetic acid (5 ml). The mixture is allowed to warm to rt, stirred for 16 h, poured into ice water, and extracted with ethyl acetate. The combined organic extract is washed with 2.5N sodium hydroxide, water and brine, dried over sodium sulfate, and concentrated in vacuo to give a residue that is triturated with hexane and a few drops of ethyl acetate. The solid is filtered to give S25-4.

### Step 5

A mixture of S25-4 (6.5 g, 28 mmol) in ethylene glycol (170 ml) is treated with crushed potassium hydroxide pellets (13 g). The mixture is heated to 190° C. for 24 h. It is poured into water and extracted with ethyl acetate. The combined organic phase is washed with brine and extracted with 1N hydrochloric acid. The combined acidic extract is washed with ethyl acetate, basified with 2.5N soldium hydoxide, and extracted with ethyl acetate. The combined organic extract is washed with brine, dried over sodium sulfate, and concentrated in vacuo to give S25-5.

### Step 6

A mixture of S25-5 (31.7 mmol) and S25-1 (31.7 mmol) in ethanol (200 ml) is stirred and heated to reflux for 56 h. The mixture is cooled, and concentrated in vacuo. The residue is dissolved in DCM and acidified with 1N HCl in ether. The solid is filtered and recrystallized to give S25-6.

### Example 26

### LC/MS Analyses

The LC/MS analyses were preformed using a AGILENT 6100 Series mass spectrometer coupled to an AGILENT 1200 Series HPLC utilizing a HALO C18 4.6×50 mm 2.7 μm column eluting at 1.8 mL/min with a solvent gradient of 5 to 95% B over 1.0 min, followed by 1.0 min at 95% B: solvent A=0.1% TFA in water; solvent B=0.1% TFA in acetonitrile. ¹H-NMR spectra were obtained on a 300 MHz VARIAN Spectrometer in CDCl₃, d⁶-DMSO or CD₃OD as indicated and chemical shifts are reported as d using the solvent peak as reference and coupling constants are reported in hertz (Hz). The LC/MS data is provided in the above tables.

### Example 27

### Functional Assays

Compounds were tested for their ability to interact with and inhibit Calcium Sensing Receptor activity using a binding assay and a FLIPR activity assay. They were also tested in rats for their ability to increase serum levels of parathyroid hormone (PTH) or calcium. Assay procedures and results are described below.

### Procedures for CaSR Binding Assay

Membrane preparations from HEK293 cells expressing the human CaSR were generated. The cells were cultured and then washed twice with 1×PBS. The cells were dissociated by adding 4 ml pre-warmed 0.25% Trypsin-EDTA, and by incubating the flask at RT for 3 minutes. 10 ml media was dispensed over the cell layer surface, and the cells were harvested into a centrifuge tube. The cells were centrifuged at 200×g for 10 minutes. The cell pellet was suspended in 30 ml of membrane preparation buffer and then homogenized on ice twice at 16000 rpm for 10 seconds. Following homogenization, the membrane was collected by centrifuge at 40000×g for 30 minutes at 4° C., washed twice with 30 ml membrane preparation buffer, and centrifuged again at 40000×g for 10 minutes. The membrane pellet was suspended in 30 ml membrane preparation buffer (50 mM Tris, pH7.0, 10 mM MgCl, Proteinase inhibitor (Roche, REF 04693116001), and then homogenized on ice once at 16000 rpm for 10 seconds. The total protein concentration was measured using a BCA kit. The membrane protein concentration was adjusted to 2.4 mg/ml and then diluted with binding buffer (10 mM HEPES, pH7.4, 130 mM NaCl, 0.4 mM CaCl₂, 5 mM MgCl₂, 1 mM EGTA, 10 mM glucose, 0.1% BSA) to 0.33 μg/μl. The final amount in the binding assay was 20 μg/well. Test compound was serially diluted manually by using BIOHIT multi-channel pipettor in 100% DMSO (10 point, 3-fold dilution of each compound from 5.0 μM in 100% DMSO). 4 μl of serially diluted compound was manually transferred into a second assay microplate that contained 196 μl of binding assay buffer to create the intermediate dilution plate. For background, 20 μl of assay buffer was added to each control well. Radio-labeled compound (EXAMPLE 25, 494 uM, 1.27 mCi/ml, apparent Kd=400 nM) was diluted in 100% DMSO to 80 μM, then diluted with binding buffer to 1.3 μM. The binding reaction used a 100 μl reaction volume consisting of 10 μl of test compound, 30 μl of diluted radio-labelled compound, and 60 μl of membrane solution (0.33 μg/μl). The reaction mixture was incubated at room temperature for one hour. The Grade GF/C filter plates were presoaked in 0.3% PEI (protect from light) for 60 minutes at room temperature. The plate was then washed three times with ice-cold wash buffer (25 mM Tris-HCl, PH7.4, 130 mM NaCl, 5 mM MgCl2, 0.4 mM CaCl2, 0.1% BSA) prior to filtration of the binding samples. The binding reaction was terminated by rapid filtration of 100 μl binding mixture through the GF/C plate. Plates were then washed 6 times with ice-cold wash buffer. Filter plates were dried with a hair dryer for 20 minutes. 60 μl/well MICROSCINT 20 (a cocktail for microplates) was added to the bottom of sealed filters, then the filters were sealed at the top with tape. Bound radioactivity was counted in a TOPCOUNT scintillation counter. Exemplary compounds bind in the 1-500 nM range.

### Procedures for CaSR FLIPR Assay

Calcilytic activity of the compound is measured in HEK293 cells stably expressing human recombinant CaSR (HEK-CaSR) using a FLIPR^{TETRA} device (Molecular Devices, Sunnyvale, Calif.) according to the manufacturer's directions. HEK-CaSR cells were maintained in cell culture medium in 5% CO₂-95% air at 37° C. to 90% confluency. The day before the assay, cells were plated at 20,000 cells/well in 96-well MATRIGEL coated black plates and incubated for 16-24 hours in a humidified tissue culture incubator at 37° C. with 5% CO₂-95% air. On the day of the assay, one vial of FLIPR Calcium 4 Assay Kit reagent Component A was equilibrated to room temperature. The content of the Component A vial was diluted by adding 10 ml dilution buffer (2×loading dye). The sample was mixed by vortexing (1-2 minutes) until the contents of the vial dissolved. The Component A vial mixture was diluted with 10 ml dilution buffer. Media was aspirated from the cells and 80 μl of loading buffer was added to each well. Plates were incubated for 1 hour at 37° C. and then placed at room temperature for 10 minutes. A two-stage method of generating 10-point serial dilutions was used. Compound was serially diluted in 100% DMSO (10 point, 3-fold dilution of each compound from 2 mM in 100% DMSO). 3 μl of serially diluted compound was transferred into a second assay microplate that contained 97 μl of TETRA dilution buffer (20 mM HEPES pH7.4, 1×HBSS without Ca and Mg, 0.4 mM CaCl₂, 0.4 mM MgSO₄, 0.5 mM MgCl₂) to create the intermediate dilution plate. After reading the base line, the compounds in 0.5% DMSO in dilution buffer were added to their respective wells as 20 μl aliquots at 6×the final drug concentration. The fluorescence signals were then read. Subsequently, 20 μl of 3.6 mM of CaCl₂ agonist (1 mM final concentration) was added to each well. For background, 20 μl of TETRA dilution buffer was added instead. The protocol consisted of a pre-addition read of 10 frames (10×1 frame/sec), a post 1^{st} addition read of 100 frames (60×1 frame/sec, 40×1 frame/3 sec), pause 3 min, a post 2^{nd} addition read of 100 frames (60×1 frame/sec, 40×1 frame/3 sec) on a FLIPR^{TETRA} Plate Reader. Results for particular compounds are provided in Tables 1-2, 4-9 and 12-24.

### Procedures for In Vivo Pharmacodynamic Effects

The objective of these studies was to assess the effect of compounds on stimulating PTH release and increasing calcium secretion in the rat. Male Sprague-Dawley rats (3-4 months old) were equipped with jugular vein cannulae (JVC) by surgical procedure. The rats were given the compound by oral gavage. Blood was sampled from the JVC at 15 minutes, 30 minutes, 60 minutes, 120 minutes, 240 minutes, 420 minutes, and 24 hrs post compound administration. Plasma was separated and used for a PTH ELISA assay and a calcium concentration assay. After the last blood sampling, the rats were sacrificed under anesthesia by inhalation of CO₂. All procedures were conducted in compliance with the Animal Welfare Act, the Guide for the Care and Use of Laboratory Animals and the recommendations of the AVMA Panel on Euthanasia.

Rat plasma samples were used for measuring serum PTH levels: The Rat Intact PTH 1-84 ELISA kit (Immutopics Cat#60-2500) was used according to the manufacturer's instructions. Total serum calcium determination was made using a calcium arsenazo III reagent (Fisher Scientific Cat # Pointe Scientific Calcium (Arsenazo III) Reagents; Mfr. No.: C7529-500; Qty.: 500 mL. Mfr. No.: C7529-500, Part #23666172) according to the manufacturer's instructions. For these experiments, 2 μl of sample was added to 198 μl of reagent, mixed, and read at 650 nm.

| Assay | EX. 5-1 | EX. 1-1 | EX. 8-24 |
|---|---|---|---|
| FLIPR CaSR (IC₅₀, nM) | 9 | 22 | 20 |
| CaSR Binding (Ki, nM) | 7 | 18 | 29 |
| Maximum Change in PTH (fold) | 8 (at | 5.8 (at | 4 (at |
| | 10 mpk) | 30 mpk) | 30 mpk) |
| Maximum Increase in Calcium (mg/dl) | 2.3 (at | 1.5 (at | 1 (at |
| | 10 mpk) | 30 mpk) | 30 mpk) |

Compound Examples 5-1, 1-1 and 8-24 have high affinity for the human calcium sensing receptor and potent antagonist activity with IC50s of 7, 18 and 29 nM, respectively. Treatment of rats with these compounds results in a robust release of PTH which reaches a maximum in the circulation within 15-30 minutes. There are also changes in serum calcium of less than 2.3 mg/dl which return to baseline by 7 hours post-dosing. This pharmacodynamic profile shows the desired characteristics with rapid and transient increases in serum PTH that are accompanied by small transient increases in serum calcium.

Pharmacokinetic evaluation in rats was also performed on these compounds as shown in the table below.

| Assay | EX. 5-1 | EX. 1-1 | EX. 8-24 |
|---|---|---|---|
| Cₘₐₓ (μM) | 0.69 | 2.4 | 0.16 |
| t_{1/2} (hour) | 1.3 | 1.7 | 1.8 |
| tₘₐₓ(hour) | 0.5 | 0.5 | 0.5 |
| F % | 7 | 24 | 7 |
| Clₚ | 52.5 | 11 | 40 |
| (mL/min/kg) | | | |

Cₘₐₓ, maximum observed plasma concentration; t_{1/2}, plasma half-life; tₘₐₓ, time to Cₘₐₓ; F, bioavailability; Clₚ, plasma clearance.

Notably, compounds of Examples 5-1, 1-1 and 8-24 have short half-lives in rats and tₘₐₓ of less than an hour. With a high Cₘₐₓ and short tₘₐₓ and t_{1/2}, it is likely that a desirable early pulsatile PTH can be achieved.

While the invention has been described and illustrated in reference to specific embodiments thereof, various changes, modifications, and substitutions can be made therein without departing from the invention. For example, alternative effective dosages may be applicable, based upon the responsiveness of the patient being treated. Likewise, the pharmacologic response may vary depending upon the particular active compound selected, formulation and mode of administration. All such variations are included within the present invention.

1. A compound of the formula (I): 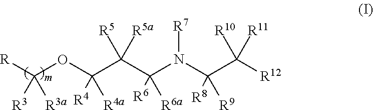
or a pharmaceutically acceptable salt thereof, wherein:
R is 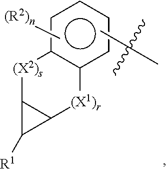
One of r and s is 0, and the other is 1,
X¹ when present, is selected from O, S, CR^{e}R^{f}, or OCR^{e}R^{f},
X² when present, is selected from O, S, CR^{e}R^{f}, or OCR^{e}R^{f},
R^{e} and R^{f} are each independently hydrogen or C₁₋₆ alkyl, wherein alkyl substituents are further optionally substituted with 1-4 halogen substituents,
R¹ is CO₂R^{a} or CONR^{b}R^{c},
R^{a} is hydrogen or C₁₋₆ alkyl,
R^{b} and R^{c} are each independently hydrogen, C₁₋₆ alkyl, or SO₂R^{d},
R^{d} is C₁₋₆ alkyl or a 3- to 6-membered cycloalkyl group optionally substituted with 1-4 substituents independently selected from: halogen or C₁₋₆alkyl, wherein alkyl substituents are further optionally substituted with 1-4 halogen substituents,
R² is halogen or C₁₋₆ alkyl, wherein alkyl substituents are further optionally substituted with 1-4 halogen substituents,
n is 0, 1, 2, or 3,
R³ and R^{3a} are each independently hydrogen or C₁₋₆ alkyl, wherein alkyl substituents are further optionally substituted with 1-4 halogen substituents,
m is 0, 1, 2, or 3,
R⁴ and R^{4a} are each independently hydrogen or C₁₋₆ alkyl, wherein alkyl substituents are further optionally substituted with 1-4 halogen substituents,
R⁵ and R^{5a} are each independently hydrogen, hydroxyl, or C₁₋₆ alkyl, wherein alkyl substituents are further optionally substituted with 1-4 halogen substituents,
R⁶ and R^{6a} are each independently hydrogen or C₁₋₆ alkyl, wherein alkyl substituents are further optionally substituted with 1-4 halogen substituents,
R⁷, R⁸ and R⁹ are each independently hydrogen or C₁₋₆ alkyl, or R⁷ is hydrogen or C₁₋₆ alkyl and R⁸ and R⁹ together form a 3- to 6-membered cycloalkyl group optionally substituted with 1-4 substituents independently selected from: halogen or C₁₋₆alkyl, or R⁷ and R⁸ together form a 5- to 6-membered heterocyclic group optionally substituted with 1-4 substituents independently selected from: halogen or C₁₋₆alkyl and R⁹ is hydrogen or C₁₋₆ alkyl,
R¹⁰ and R¹¹ are each independently hydrogen or C₁₋₆ alkyl, wherein alkyl substituents are further optionally substituted with 1-4 halogen substituents, or R¹⁰ and R¹¹ together form an oxo group, and
R¹² is a 6- to 10-membered aryl group, a 5- to 10-membered heteroaryl group, a 5- to 7-membered cycloalkyl group, a 5- to 7-membered heterocyclic group, —(CH₂)₀₋₃—O—(CH₂)₀₋₁-6- to 10-membered aryl, —(CH₂)₁₋₃-6- to 10-membered aryl, —(CH₂)₀₋₃—S—(CH₂)₀₋₁-6- to 10-membered aryl, wherein aryl, heteroaryl, cycloalkyl, heterocyclic group optionally substituted with 1-4 substituents independently selected from: halogen, hydroxyl, oxo, C₁₋₆alkyl, C₁₋₆alkylOC₁₋₆alkyl, C₁₋₆alkoxy, CN, C(O)₁₋₂C₁₋₆alkyl, C₁₋₆alkylC(O)₁₋₂C₁₋₆alkyl, or S(O)₀₋₂C₁₋₆alkyl, wherein alkyl substituents are further optionally substituted with 1-4 halogen substituents.

2. The compound of claim 1 wherein:
R is 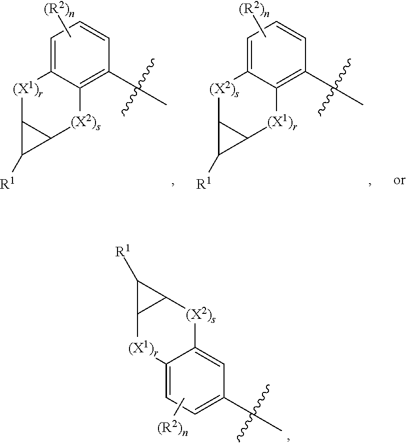
X¹ is O, S, CR^{e}R^{f}, or OCR^{e}R^{f} where the O atom in OCR^{e}R^{f} is directly attached to the phenyl ring of the R group, and R^{e} and R^{f} as defined in claim 1,
r is 1, and
s is 0, or a pharmaceutically acceptable salt thereof.

3. The compound of claim 1 wherein:
R is 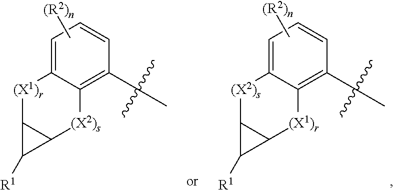
X¹ is O, S, CR^{e}R^{f}, or OCR^{e}R^{f} where the O atom in OCR^{e}R^{f} is directly attached to the phenyl ring of the R group, and R^{e} and R^{f} as defined in claim 1,
r is 1, and
s is 0, or a pharmaceutically acceptable salt thereof.

4. The compound of claim 1 wherein:
R is 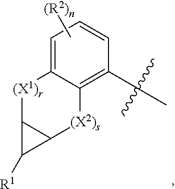
X¹ is O, S, CR^{e}R^{f} or OCR^{e}R^{f} where the O atom in OCR^{e}R^{f} is directly attached to the phenyl ring of the R group, and R^{e} and R^{f} as defined in claim 1,
r is 1, and
s is 0, or a pharmaceutically acceptable salt thereof.

5. The compound of claim 1 wherein:
X¹ when present, is selected from O or CH₂, and
X² when present, is selected from O or CH₂, or a pharmaceutically acceptable salt thereof.

6. The compound of claim 1 wherein:
R¹ is CO₂H, CO₂C₁₋₆alkyl, or CONHS(O)₂R^{d}, and
R^{d} is a 3- to 6-membered cycloalkyl group optionally substituted with 1-4 substituents independently selected from: halogen or C₁₋₆alkyl, or a pharmaceutically acceptable salt thereof.

7. The compound of claim 1 wherein R² is halogen or CH₃, or a pharmaceutically acceptable salt thereof.

8. The compound of claim 1 wherein R² is CH₃, or a pharmaceutically acceptable salt thereof.

9. The compound of claim 1 wherein R³ and R^{3a} are each independently hydrogen or CH₃, or a pharmaceutically acceptable salt thereof.

10. The compound of claim 1 wherein one of R³ and R^{3a} is hydrogen, and the other is CH₃, or a pharmaceutically acceptable salt thereof.

11. The compound of claim 1 wherein R⁴ and R^{4a} are each independently hydrogen or CH₃, or a pharmaceutically acceptable salt thereof.

12. The compound of claim 1 wherein one of R⁵ and R^{5a} is hydroxyl, and the other is hydrogen, or a pharmaceutically acceptable salt thereof.

13. The compound of claim 1 wherein R⁶ and R^{6a} are both hydrogen, or a pharmaceutically acceptable salt thereof.

14. The compound of claim 1 wherein R⁷, R⁸ and R⁹ are each independently hydrogen or CH₃, or R⁷ is hydrogen or CH₃ and R⁸ and R⁹ together form a 3- to 5-membered cycloalkyl group optionally mono- or di-substituted with CH₃, or R⁷ and R⁸ together form a 5- to 6-membered heterocyclic group optionally mono- or di-substituted with CH₃ and R⁹ is hydrogen or CH₃, or a pharmaceutically acceptable salt thereof.

15. The compound of claim 1 wherein R⁷ is hydrogen, while R⁸ and R⁹ are both CH₃, or a pharmaceutically acceptable salt thereof.

16. The compound of claim 1 wherein R¹⁰ and R¹¹ are both hydrogen, or a pharmaceutically acceptable salt thereof.

17. The compound of claim 1 wherein R¹² is phenyl group, naphthyl group, indanyl group, quinolyl group, benzothienyl group, dihydrobenzothienyl group, benzofuranyl group, dihydrobenzofuranyl group, benzodioxolanyl group, benzodioxanyl group, tetrahydroisoquinolyl group, —(CH₂)₀₋₁—S—(CH₂)₀₋₁-phenyl, —(CH₂)₀₋₁—O—(CH₂)₀₋₁-phenyl, or —(CH₂)₂-phenyl, optionally substituted with 1-4 substituents independently selected from: halogen, CH₃, CF₃, OCH₃, or S(O)₀₋₁CH₃, or a pharmaceutically acceptable salt thereof.

18. A compound which is:
5-(1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
5-(1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
5-(1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
3-(1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
3-(1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
6-(1-((R)-3-((1-(4-chloro-3-fluorophenyl)-2-methylpropan-2-yl)amino)-2-hydroxypropoxy)ethyl)-3-methyl-1a,6b-dihydro-1H-cyclopropa[b]benzofuran-1-carboxylic acid;
6-(1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
6-(1-((R)-3-((1-(2,3-dihydro-1H-inden-2-yl)-2-methylpropan-2-yl)amino)-2-hydroxypropoxy)ethyl)-3-methyl-1a,6b-dihydro-1H-cyclopropa[b]benzofuran-1-carboxylic acid;
6-((R)-1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
3-(1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
5-((R)-3-((1-(4-chloro-3-fluorophenyl)-2-methylpropan-2-yl)amino)-2-hydroxypropoxy)-1a,6b-dihydro-1H-cyclopropa[b]benzofuran-1-carboxylic acid;
5-((R)-2-hydroxy-3-((2-methyl-1-(naphthalen-2-yl)propan-2-yl)amino)propoxy)-1a,6b-dihydro-1H-cyclopropa[b]benzofuran-1-carboxylic acid;
5-((R)-3-((1-(2,3-dihydro-1H-inden-2-yl)-2-methylpropan-2-yl)amino)-2-hydroxypropoxy)-1a,6b-dihydro-1H-cyclopropa[b]benzofuran-1-carboxylic acid;
6-(1-((R)-3-((1-(4-chloro-3-fluorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
6-(1-((R)-3-((1-(2,3-dihydro-1H-inden-2-yl)-2-methylpropan-2-yl)amino)-2-hydroxypropoxy)ethyl)-3-methyl-1a,6b-dihydro-1H-cyclopropa[b]benzofuran-1-carboxylic acid;
6-(1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-3-((1-(4-chloro-3-fluorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(2,3-dihydro-1H-inden-2-yl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-3-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1R,1aR,6bS)-3-((R)-1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1R,1aR,6bS)-3-((R)-1-((R)-3-((1-(3,4-dimethylphenyl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(4-chloro-2-fluorophenyl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-1-(naphthalen-1-yl)propan-2-yl)amino)propoxy)ethyl)-3-methyl-1a,6b-dihydro-1H-cyclopropa[b]benzofuran-1-carboxylic acid;
(1R,1aR,6bS)-6-((R)-1-((R)-3-((1-(2,3-dihydro-1H-inden-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-3-((1-(4-chloro-3-fluorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
(1R,1aR,6bS)-6-((R)-1-((R)-3-((1-(2,3-dihydro-1H-inden-2-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-6-((R)-1-((R)-3-((1-(2-chloro-4-methoxyphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-3-((R)-1-((R)-3-((1-(2,3-dihydro-1H-inden-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(3-fluoro-4-(methylthio)phenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(2,3-dihydro-1H-inden-5-yl)-2-methylpropan-2-yl)amino)-2-hydroxypropoxy)ethyl)-1a,6b-dihydro-1H-cyclopropa[b]benzofuran-1-carboxylic acid;
6-(1-((R)-2-hydroxy-3-((2-methyl-1-(naphthalen-2-yl)propan-2-yl)amino)propoxy)ethyl)-1a,6b-dihydro-1H-benzo[b]cyclopropa[d]thiophene-1-carboxylic acid;
(1S,1aS,6bR)-3-((R)-1-((R)-3-((1-(benzo[b]thiophen-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydrobenzo[b][1,4]dioxin-6-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3-fluoro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((1R)-1-((2R)-3-((1-(3-fluoro-4-(methylsulfinyl)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydrobenzo[b]thiophen-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzo[b]thiophen-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-4-(phenylthio)butan-2-yl)amino)propoxy)ethyl)-3-methyl-1a,6b-dihydro-1H-cyclopropa[b]benzofuran-1-carboxylic acid;
(1R,1aR,6aS)-2-((R)-1-((R)-2-hydroxy-3-((2-methyl-1-(naphthalen-2-yl)propan-2-yl)amino)propoxy)ethyl)-1,1a,6,6a-tetrahydrocyclopropa[a]indene-1-carboxylic acid;
(1S,1aS,6bR)-6-((R)-1-((R)-3-((1-(3-chloro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-5-chloro-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-5-chloro-6-((R)-1-((R)-2-hydroxy-3-((2-methyl-1-(naphthalen-2-yl)propan-2-yl)amino)propoxy)ethyl)-3-methyl-1a,6b-dihydro-1H-cyclopropa[b]benzofuran-1-carboxylic acid;
3-fluoro-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
3-chloro-6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-5-fluoro-3-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6bR)-5-chloro-3-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylic acid;
(1S,1aS,6aR)-2-((R)-1-((R)-2-hydroxy-3-((2-methyl-1-(naphthalen-2-yl)propan-2-yl)amino)propoxy)ethyl)-1,1a,6,6a-tetrahydrocyclopropa[a]indene-1-carboxylic acid;
(1R,1aR,6aS)-5-((R)-1-((R)-2-hydroxy-3-((2-methyl-1-(naphthalen-2-yl)propan-2-yl)amino)propoxy)ethyl)-1,1a,6,6a-tetrahydrocyclopropa[a]indene-1-carboxylic acid;
(1S,1aS,6aR)-5-((R)-1-((R)-2-hydroxy-3-((2-methyl-1-(naphthalen-2-yl)propan-2-yl)amino)propoxy)ethyl)-1,1a,6,6a-tetrahydrocyclopropa[a]indene-1-carboxylic acid;
7-(1-((R)-2-hydroxy-3-((2-methyl-1-(naphthalen-2-yl)propan-2-yl)amino)propoxy)ethyl)-2,2,4-trimethyl-1,1a,2,7b-tetrahydrocyclopropa[c]chromene-1-carboxylic acid;
(1S,1aS,6bR)-ethyl 6-((R)-1-((R)-2-hydroxy-3-((2-methyl-1-(naphthalen-2-yl)propan-2-yl)amino)propoxy)ethyl)-1a,6b-dihydro-1H-cyclopropa[b]benzofuran-1-carboxylate;
(1S,1aS,6bR)-ethyl 6-((R)-1-((R)-3-((1-(3-fluoro-4-(methylthio)phenyl)-2-methylpropan-2-yl)amino)-2-hydroxypropoxy)ethyl)-3-methyl-1a,6b-dihydro-1H-cyclopropa[b]benzofuran-1-carboxylate;
(1S,1aS,6bR)-6-((R)-1-((R)-2-hydroxy-3-((2-methyl-1-(naphthalen-2-yl)propan-2-yl)amino)propoxy)ethyl)-N-((1-methylcyclopropyl)sulfonyl)-1a,6b-dihydro-1H-cyclopropa[b]benzofuran-1-carboxamide;
(1S,1aS,6bR)-3-((R)-1-((R)-2-hydroxy-3-((2-methyl-1-(naphthalen-2-yl)propan-2-yl)amino)propoxy)ethyl)-N-((1-methylcyclopropyl)sulfonyl)-1a,6b-dihydro-1H-cyclopropa[b]benzofuran-1-carboxamide;
(1S,1aS,6bR)-6-((R)-1-((R)-3-((1-(3-fluoro-4-methylphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3,4-dimethylphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzo[b]thiophen-2-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzofuran-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((1R)-1-((2R)-3-(2-benzylpyrrolidin-1-yl)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(2,3-dihydro-1H-inden-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzo[d][1,3]dioxol-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzo[b]thiophen-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(2-fluoro-4-methoxyphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3,4-dichlorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-5-phenylpentan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-4-phenoxybutan-2-yl)amino)propoxy)ethyl)-3-methyl-1a,6b-dihydro-1H-cyclopropa[b]benzofuran-1-carboxylic acid;
(1S,1aS,6bR)-6-((1R)-((2R)-3-(2-benzyl-2-methylpyrrolidin-1-yl)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3,4-difluorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(4-fluoro-3-(trifluoromethyl)phenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(2,4,5-trifluorophenyl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(2,4,6-trifluorophenyl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(4-(methylthio)phenyl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(quinolin-6-yl)propan-2-yl)amino)propoxy)ethyl)-3-methyl-1a,6b-dihydro-1H-cyclopropa[b]benzofuran-1-carboxylic acid;
(1S,1aS,6bR)-6-((R)-1-((R)-3-((1-(1,1-dioxido-2,3-dihydrobenzo[b]thiophen-5-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(benzylthio)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((4-((4-fluorophenyl)thio)-2-methylbutan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-((4-fluorophenyl)thio)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(3-chloro-4-(methylthio)phenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-2-hydroxy-3-((2-methyl-1-(3-methyl-4-(methylthio)phenyl)propan-2-yl)amino)propoxy)ethyl)-3-methyl-1a,6b-dihydro-1H-cyclopropa[b]benzofuran-1-carboxylic acid;
(1R,1aR,6bS)-6-((R)-1-((R)-3-((1-(3-fluoro-4-methylphenyl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-3-((1-(3-fluoro-4-methoxyphenyl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-2-hydroxy-3-((1-(naphthalen-2-ylmethyl)cyclopropyl)amino)propoxy)ethyl)-3-methyl-1a,6b-dihydro-1H-cyclopropa[b]benzofuran-1-carboxylic acid;
(1R,1aR,6bS)-6-((R)-1-((R)-2-hydroxy-3-((1-(naphthalen-2-ylmethyl)cyclobutyl)amino)propoxy)ethyl)-3-methyl-1a,6b-dihydro-1H-cyclopropa[b]benzofuran-1-carboxylic acid;
(1R,1aR,6bS)-6-((R)-1-((R)-3-((1-(2,4-dichlorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1R,1aR,6bS)-6-((R)-1-((R)-3-((1-(3,4-dichlorophenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-6-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydro-1H-inden-2-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(4-chloro-3-fluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(3,4-dimethylphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(benzofuran-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(benzo[d][1,3]dioxol-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2,3-dihydrobenzo[b][1,4]dioxin-6-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(2-fluoro-4-methoxyphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-3-((1-(3,4-dichlorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-1-(4-(methylthio)phenyl)propan-2-yl)amino)propoxy)ethyl)-1a,6b-dihydro-1H-cyclopropa[b]benzofuran-1-carboxylic acid;
(1S,1aS,6bR)-6-((R)-1-((R)-3-((1-(2-chloro-4-methoxyphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-4-(phenylthio)butan-2-yl)amino)propoxy)ethyl)-1a,6b-dihydro-1H-cyclopropa[b]benzofuran-1-carboxylic acid;
(1S,1aS,6bR)-6-((R)-1-((R)-2-hydroxy-3-((2-methyl-1-(3-methyl-4-(methylthio)phenyl)propan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(3-fluoro-4-methylphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(3,4-dimethylphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(benzofuran-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(benzo[d][1,3]dioxol-5-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(2,3-dihydrobenzo[b][1,4]dioxin-6-yl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(2-fluoro-4-methylphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(2-fluoro-4-methoxyphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(3,4-dichlorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-2-hydroxy-3-((2-methyl-5-phenylpentan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(3,4-difluorophenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((1R)-1-((2R)-2-hydroxy-3-((2-methyl-1-(4-(methylsulfinyl)phenyl)propan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(3-fluoro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((1R)-1-((2R)-3-((1-(3-fluoro-4-(methylsulfinyl)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-3-((1-(2-chloro-4-methoxyphenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-2-hydroxy-3-((2-methyl-4-(phenylthio)butan-2-yl)amino)propoxy)ethyl)-1a,6b-dihydro-1H-cyclopropa[b]benzofuran-1-carboxylic acid;
(1S,1aS,6bR)-3-((R)-1-((R)-3-((1-(3-chloro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1S,1aS,6bR)-3-((R)-1-((R)-2-hydroxy-3-((2-methyl-1-(3-methyl-4-(methylthio)phenyl)propan-2-yl)amino)propoxy)ethyl)-1a,6b-dihydro-1H-cyclopropa[b]benzofuran-1-carboxylic acid;
(1R,1aR,6bS)-3-((R)-1-((R)-3-((1-(benzyloxy)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(3,4-dihydroisoquinolin-2(1H)-yl)-2-methyl-1-oxopropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(3,4-dihydroisoquinolin-2(1H)-yl)-2-methylpropan-2-yl)amino)-2-hydroxypropoxy)ethyl)-1a,6b-dihydro-1H-cyclopropa[b]benzofuran-1-carboxylic acid;
(1R,1aR,6bS)-3-((R)-1-((R)-3-((1-(3-fluoro-4-(methylthio)phenyl)-2-methylpropan-2-yl)amino)-2-hydroxypropoxy)ethyl)-1a,6b-dihydro-1H-cyclopropa[b]benzofuran-1-carboxylic acid;
(1R,1aR,6aS)-2-((R)-1-((R)-3-((1-(benzofuran-5-yl)-2-methylpropan-2-yl)amino)-2-hydroxypropoxy)ethyl)-1,1a,6,6a-tetrahydrocyclopropa[a]indene-1-carboxylic acid;
(1R,1aR,6aS)-2-((R)-1-((R)-3-((1-(2,3-dihydro-1H-inden-5-yl)-2-methylpropan-2-yl)amino)-2-hydroxypropoxy)ethyl)-1,1a,6,6a-tetrahydrocyclopropa[a]indene-1-carboxylic acid;
(1R,1aR,6aS)-2-((R)-1-((R)-3-((1-(benzo[b]thiophen-5-yl)-2-methylpropan-2-yl)amino)-2-hydroxypropoxy)ethyl)-1,1a,6,6a-tetrahydrocyclopropa[a]indene-1-carboxylic acid;
(1R,1aR,6aS)-2-((R)-1-((R)-3-((1-(3-fluoro-4-(methylthio)phenyl)-2-methylpropan-2-yl)amino)-2-hydroxypropoxy)ethyl)-1,1a,6,6a-tetrahydrocyclopropa[a]indene-1-carboxylic acid;
(1S,1aS,6bR)-6-((R)-1-((R)-3-((1-(benzofuran-5-yl)-2-methylpropan-2-yl)amino)-2-hydroxypropoxy)ethyl)-5-chloro-3-methyl-1a,6b-dihydro-1H-cyclopropa[b]benzofuran-1-carboxylic acid;
(1S,1aS,6bR)-6-((R)-1-((R)-3-((1-(benzo[b]thiophen-5-yl)-2-methylpropan-2-yl)amino)-2-hydroxypropoxy)ethyl)-5-chloro-3-methyl-1a,6b-dihydro-1H-cyclopropa[b]benzofuran-1-carboxylic acid;
(1S,1aS,6bR)-5-chloro-6-((R)-1-((R)-3-((1-(2,3-dihydrobenzo[b][1,4]dioxin-6-yl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-5-chloro-6-((R)-1-((R)-3-((1-(2-fluoro-4-methoxyphenyl)-2-methylpropan-2-yl)amino)-2-hydroxypropoxy)ethyl)-3-methyl-1a,6b-dihydro-1H-cyclopropa[b]benzofuran-1-carboxylic acid;
(1S,1aS,6bR)-5-chloro-6-((R)-1-((R)-3-((1-(3-fluoro-4-(methylthio)phenyl)-2-methylpropan-2-yl)amino)-2-hydroxypropoxy)ethyl)-3-methyl-1a,6b-dihydro-1H-cyclopropa[b]benzofuran-1-carboxylic acid; or
a pharmaceutically acceptable salt thereof.

19. A pharmaceutical composition comprising a compound or pharmaceutically acceptable salt of claim 1, and a pharmaceutically acceptable carrier.

20. A method for the treatment of osteoporosis comprising administering to an individual a pharmaceutical composition comprising the compound or pharmaceutically acceptable salt of claim 1.
